SEQUENCE LISTING (21 sequences)

SEQ ID NO: 1 - DNA, 978 bp, Homo sapiens
atgaccatggaatctggagccgagaaccagcagagtggagatgcagctgtaacagaagctgaaaaccaagaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatca

SEQ ID NO: 2 - protein, 327 aa, Homo sapiens
MTMESGAENQQSGDAAVTEAENQEMTVQAQPQIATLAQVSMPAAHATSSAPTVTLVQLPNGQTVQVHGVIQAAQPSVIQSPQVQTVQISTIAESEDSQESVDSVTDSQKRREILSRRPSYRKILNDLSSDAPGVPRIEEEKSEEETSAPAITTVTVPTPIYQTSSGQYIAITQGGAIQLANNGTDGVQGLQTLTMTNAAATQPGTTILQYAQTTDGQQILVPSNQVVVQAASGDVQTYQIRTAPTSTIAPGVVMASSPALPTQPAEEAARKREVRLMKNREAARECRRKKKEYVKCLENRVAVLENQNKTLIEELKALKDLYCHKSZ

SEQ ID NO: 3 - DNA, 1022 bp, Rattus rattus
atgaccatggactctggagcagacaaccagcagagtggagatgctgctgtaacagaagctgaaagtcaagaaatgacagttcaagcccagccacagattgccacattagcccaggtatccatgccagcagctcatgcgacgtcatctgctcccactgtaaccttagtgcagctgcccaatgggcagacagtccaggtccatggggtcatccaggcggcccagccatcagttattcagtctccacaagtccaaacagttcagtcttggtctaaggacttaaaaagacttttctccggaactcagatttcaactattgcagaaagtgaagattcacaggagtctgtggatagtgtaactgattcccaaaaacgaagggaaatcctttcaaggaggccttcctacaggaaaattttgaatgacttatcttctgatgcaccaggggtgccaaggattgaagaagaaaaatcagaagaagagacttcagcccctgccatcaccactgtaacagtgccaaccccgatttaccaaactagcagtgggcagtatattgccattacccagggaggagcaatacagctggctaacaatggtaccgatggggtacagggcctgcagacattaaccatgaccaatgcagctgccactcagccgggtactaccattctacaatatgcacagaccactgatggacagcagattctagtgcccagcaaccaagttgttgttcaagctgcctctggtgatgtacaaacataccagattcgcacagcacccactagcaccattgccctggagttgttatggcgtcctccccagcacttcctacacagcctgctgaagaagcagcacgaaagagagaggttcgtctaatgaagaacagggaagcagcaagagaatgtcgtagaaagaagaaagaatatgtgaaatgtttagagaacagagtggcagtgcttgaaaaccaaaacaaaacattgattgaggagctaaaagcacttaaggacctttactgccacaagtcagat

SEQ ID NO: 4 - protein, 342 aa, Rattus rattus
MTMESGADNQQSGDAAVTEAESQEMTVQAQPQIATLAQVSMPAAHATSSAPTVTLVQLPNGQTVQVHGVIQAAQPSVIQSPQVQTVQSWSKDLKRLFSGTQISTIAESEDSQESVDSVTDSQKRREILSRRPSYRKILNDLSSDAPGVPRIEEEKSEEETSAPAITTVTVPTPIYQTSSGQYIAITQGGAIQLANNGTDGVQGLQTLTMTNAAATQPGTTILQYAQTTDGQQILVPSNQVVVQAASGDVQTYQIRTAPTSTIAPGVVMASSPALPTQPAEEAARKREVRLMKNREAARECRRKKKEYVKCLENRVAVLENQNKTLIEELKALKDLYCHKSDZ

SEQ ID NO: 5 - DNA, 627 bp, Homo sapiens
atggcacccaagaagaagcggaaggtcccaggtgtaccgagctcgaattgcatcgatacggcccccccgaccgatgtcagcctgggggacgagctccacttagacggcgaggacgtggcgatggcgcatgccgacgcgcatgacgatttcgatctggacatgttgggggacggggattccccggggccgggatttaccccccacgactccgccccctacggcgctctggatatggccgacttcgagtttgagcagatgtttaccgatgcccttggaattgacgagtacggtgggatcgattggatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcag

SEQ ID NO: 6 - protein, 210 aa, Homo sapiens
MAPKKKRKVPGVPSSNCIDTAPPTDVSLGDELHLDGEDVAMAHADAHDDFDLDMLGDGDSPGPGFTPHDSAPYGALDMADFEFEQMFTDALGIDEYGGIDWILVPSNQVVVQAASGDVQTYQIRTAPTSTIAPGVVMASSPALPTQPAEEAARKREVRLMKNREAARECRRKKKEYVKCLENRVAVLENQNKTLIEELKALKDLYCHKSZ

SEQ ID NO: 7 - DNA, 1016 bp, Homo sapiens
atgaccatggaatctggagccgagaaccagcagagtggagatgcagctgtaacagaagctgaaaaccaagaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagattaatttgggatttatcaagctggggtaccgaattc

SEQ ID NO: 8 - protein, 328 aa, Homo sapiens
MTMESGAENQQSGDAAVTEAENQEMTVQAQPQIATLAQVSMPAAHATSSAPTVTLVQLPNGQTVQVHGVIQAAQPSVIQSPQVQTVQISTIAESEDSQESVDSVTDSQKRREILSRRPSYRKILNDLSSDAPGVPRIEEEKSEEETSAPAITTVTVPTPIYQTSSGQYIAITQGGAIQLANNGTDGVQGLQTLTMTNAAATQPGTTILQYAQTTDGQQILVPSNQVVVQAASGDVQTYQIRTAPTSTIAPGVVMASSPALPTQPAEEAARKREVRLMKNREAARECRRKKKEYVKCLENRVAVLENQNKTLIEELKALKDLYCHKSDZ

SEQ ID NO: 9 - DNA, 8 bp, Artificial Sequence
  Description of Artificial Sequenceconsensus
ttacgtca

SEQ ID NO: 10 - DNA, 20 bp, Homo sapiens
ggccccttacgtcagaggcg

SEQ ID NO: 11 - DNA, 19 bp, Homo sapiens
aaaacatgactcagaggaa

SEQ ID NO: 12 - DNA, 20 bp, Homo sapiens
cagccaagctgtcagcccat

SEQ ID NO: 13 - DNA, 20 bp, Homo sapiens
gggggctgaggaaatactga

SEQ ID NO: 14 - DNA, 20 bp, Homo sapiens
tgtattttactgcaatttta

SEQ ID NO: 15 - DNA, 20 bp, Homo sapiens
tgggcgttgcgccacgatct

SEQ ID NO: 16 - DNA, 20 bp, Homo sapiens
gcgggctggcgtcacccgcg

SEQ ID NO: 17 - DNA, 20 bp, Homo sapiens
gggcgtgcgcgtcagctggg

SEQ ID NO: 18 - DNA, 60 bp, Artificial Sequence
  Description of Artificial Sequenceprobe
gtaatcatcgaagttttcactggagacaagcttccaggttcccacaaaggcatcacacat

SEQ ID NO: 19 - DNA, 31183 bp, Adenovirus type 37
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcaggatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 20 - DNA, 816 bp, Homo sapiens
  CDS (1)..(816)
atggaagattcccacaagagtaccacgtcagagacagcacctcaacctggttcagcagttcagggagctcacatttctcatattgctcaacaggtatcatctttatcagaaagtgaggagtcccaggactcatccgacagcataggctcctcacagaaagcccacgggatcctagcacggcgcccatcttacagaaaaattttgaaagacttatcttctgaagatacacggggcagaaaaggagacggagaaaattctggagtttctgctgctgtcacttctatgtctgttccaactcccatctatcagactagcagcggacagtacattgccattgccccaaatggagccttacagttggcaagtccaggcacagatggagtacagggacttcagacattaaccatgacaaattcaggcagtactcagcaaggtacaactattcttcagtatgcacagacctctgatggacagcagatacttgtgcccagcaatcaggtggtcgtacaaactgcatcaggagatatgcaaacatatcagatccgaactacaccttcagctacttctctgccacaaactgtggtgatgacatctcctgtgactctcacctctcagacaactaagacagatgacccccaattgaaaagagaaataaggttaatgaaaaacagagaagctgctcgagaatgtcgcagaaagaagaaagaatatgtgaaatgcctggaaaaccgagttgcagtcctggaaaatcaaaataaaactctaatagaagagttaaaaactttgaaggatctttattccaataaaagtgtttga

SEQ ID NO: 21 - protein, 271 aa, Homo sapiens
MEDSHKSTTSETAPQPGSAVQGAHISHIAQQVSSLSESEESQDSSDSIGSSQKAHGILARRPSYRKILKDLSSEDTRGRKGDGENSGVSAAVTSMSVPTPIYQTSSGQYIAIAPNGALQLASPGTDGVQGLQTLTMTNSGSTQQGTTILQYAQTSDGQQILVPSNQVVVQTASGDMQTYQIRTTPSATSLPQTVVMTSPVTLTSQTTKTDDPQLKREIRLMKNREAARECRRKKKEYVKCLENRVAVLENQNKTLIEELKTLKDLYSNKSV

(19) United States
(12) Patent Application Publication
(10) Pub. No.: US 2004/0097454 A1
(43) Pub. Date: May 20, 2004

(54) Method for modulation of cell phenotype

(75) Inventors: Jane E. Reusch, Denver, CO (US); Dwight J. Klemm, Highlands Ranch, CO (US)

Correspondence Address: SHERIDAN ROSS PC, 1560 BROADWAY, SUITE 1200, DENVER, CO, 80202

(73) Assignees: University Technology Corporation; National Jewish Medical and Research Center; U.S. Government as represented by the Department of Veterans Affairs

(21) Appl. No.: 10/431,598

(22) Filed: May 6, 2003

Related U.S. Application Data
(63) Continuation of application No. 09/420,060, filed on Oct. 18, 1999, now abandoned.

(51) Int. Cl. A61K 48/00; C12Q 1/68

(52) U.S. Cl. 514/44; 435/6

(57) ABSTRACT

Described is a method for modulating the phenotype of a cell, and particularly, of a target cell in a patient who has or is at risk of developing a disease or condition in which is associated with dysregulation of cellular phenotype. The method includes administration of a recombinant nucleic acid molecule encoding a protein having CREB biological activity or dominant negative CREB biological activity to a patient, in such a manner that the protein is expressed in a target cell of a patient and is sufficient to modulate the phenotype of the target cell. Such a method is particularly useful in patients who have, or at risk of developing, diabetes, obesity, macrovascular disease, heart failure, osteoarthritis, and neural diseases and conditions.

### GOVERNMENT RIGHTS

This invention was made in part with government support under Public Health Service Grants GM47117 and DK53969 and DKO235, Veterans Administration Merit and Career Development Awards, and Veterans Administration Research Enhancement Award in Neurodegeneration and Apoptosis. The government may have certain rights to this invention.

### FIELD OF THE INVENTION

The present invention generally relates to a method for modulation of cell phenotype. In particular, the present invention relates to a method to modulate cell phenotype in a target cell by administration of a recombinant nucleic acid molecule encoding a cyclic AMP responsive element binding protein (CREB).

### BACKGROUND OF THE INVENTION

It has been estimated that 26% of Americans are overweight (1998), with 5-14% of men and 7-24% of women considered obese depending on the definition employed (Abraham et al., 1980, Am. J. Clin. Nutr., 33:364-369; Bray, 1979, In Obesity in America. DHEW Publication No. (NIH) 79-359., G. A. Bray, ed. (Washington, D.C.: Government Printing Office), pp. 1-19; Foreyt et al., 1998, Int'l J. Fertility and Womens Med., 43:111-116; Pi-Sunyer et al., 1999, J. Clin. Endo. Metab., 84:3-7; Slyper, 1998, Pediatrics 102, e4.) Similar or even higher estimates for the prevalence of obesity have been reported in other countries (McIntyre, 1998, J. Royal Soc. Health, 118:76-84). Weight gain and obesity occur when energy intake by an individual exceeds the rate of energy expenditure (Foster, 1992, Eating disorders: obesity, anorexia nervosa, and bulemia nervosa. In William's Textbook of Endocrinology, J. D. Wilson and D. W. Foster, eds. (Philadelphia, Pa.: W.B. Saunders Company), pp. 1335-1365. Energy intake and expenditure are in turn determined by multiple, interacting factors ranging from dietary composition, and feeding and exercise habits, to physiologic factors and biochemical pathways that modulate lipid and energy metabolism (Spiegelman et al., 1996, Cell 87:377-389). At the cellular level obesity was originally considered a hypertrophic disease resulting from an increase in fat cell size or volume (Hirsch et al., 1976, Clin. Endo. Metab., 5:299-311). However, several studies have demonstrated a hyperplastic component to obesity. For example, sequential biopsies in children indicate that fat cell numbers increase when body fat reaches 25% of total weight (Ginsberg-Fellner et al., 1981, Pediatr. Res., 15:1381-1389; Knittle et al., 1979, J. Clin. Invest., 63:239-246). Similarly, obese adults have increased numbers of fat cells (Hirsch et al., 1976, Clin. Endo. Metab., 5:299-311), and preadipocytes from obese subjects proliferate more rapidly in culture than cells from lean individuals (Hirsch et al., 1976, Clin. Endo. Metab., 5:299-311; Roncari et al., 1981, Metabolism, 30:425-427). New fat cells could arise from a pre-existing population of undifferentiated progenitor cells, or through the dedifferentiation of adipocytes to preadipocytes which then proliferate and redifferentiate into mature adipocytes. In either case, the generation of new fat cells demonstrates the crucial role of adipocyte proliferation and differentiation in the development of obesity.

Several factors including the nuclear hormone receptor, Peroxisome Proliferator Activated Receptor gamma 2 (PPARγ2), members of the CCAAT/Enhancer Binding Protein (CEBP) family of transcription factors, and Adipocyte Determination/Differentiation factor 1 (ADD1/SREBP) appear to play paramount roles in adipocyte differentiation (MacDougald and Lane, 1995; Spiegelman and Flier, 1996). Ectopic expression of PPARγ2 has been shown to drive the differentiation of preadipocytes to mature adipocytes in the presence of PPAR ligands, and PPARγ2 has been shown to bind the promoters of several adipocyte-specific genes as a heterodimer with the cis-retinoic acid receptor alpha (RXRα) (Tontonoz et al., 1994, Nucleic Acids Res. 22:5628-5634; Tontonoz et al., 1995, Mol. Cell. Biol., 15:351-357). CEBP β, which is expressed early in the adipocyte differentiation program, has likewise been shown to promote the differentiation of fibroblasts to adipocytes (Yeh et al., 1995, Genes & Development, 9:168-181), and increase the expression of PPARγ2 (Wu et al., 1995, Genes and Development, 9:2350-2356). CEBP α is expressed relatively late in adipogenesis and appears to accelerate or potentiate the differentiation process as well as simulate the expression of certain adipocyte-specific genes (MacDougald et al., 1995, Annu. Rev. Biochem., 64:345-373). While expressed late in adipocyte development, overexpression of CEBP α in fibroblasts will induce their differentiation to mature fat cells (Freytag et al., 1994, Genes and Develop., 8:1654-1663). Expression of ADD1/SREBP1 alone is not sufficient to induce adipogenesis, but this factor has been shown to stimulate the expression of genes involved with lipid metabolism (Kim et al., 1996, Genes Dev., 10:1096-1107) and increase the percentage of cells that undergo adipocyte differentiation under conditions that favor adipogenesis. Interestingly, all the aforementioned factors are undetectable or expressed at very low levels in preadipocytes, and their expression increases only after the induction of adipogenesis. This suggests that the expression of these factors and induction of adipogenesis is under the control of a(n) unidentified factor(s) present in undifferentiated preadipocytes.

Obesity contributes to an increased rate of mortality (Drenick et al., 1980, JAMA, 243:443-445) by virtue of its role in the development of cardiovascular disease, diabetes, pulmonary dysfunction, and gall stones (Black et al., 1983, J. Royal College Physicians, 17:5-65; Bray, 1979, In Obesity in America. DHEW Publication No. (NIH) 79-359., G. A. Bray, ed. (Washington, D.C.: Government Printing Office), pp. 1-19). Diabetes will affect an estimated 16 million Americans by the year 2000 (Strandberg et al., Eur J Biochem 176:609-16,1988). Cardiovascular disease is the leading cause of death in the United States and the prevalence is radically increased in individuals with diabetes (Shimomura et al., 1998, J. Neurochem., 70:1029-1034). People with diabetes have a 2-3 fold increased risk of the development of cardiovascular disease. Once individuals with diabetes have a myocardial infarction they are twice as likely as non-diabetics to experience a second event or to develop congestive heart failure. Despite intervention with blood pressure control, aspirin, b blockers, and HMG CoA reductase inhibitors 75% of all mortality in individuals with diabetes is secondary to cardiovascular disease.

Macrovascular disease including atherosclerosis, acute MI, stroke, and amputation from peripheral vascular disease is responsible for the majority of the morbidity and mortality in individuals with diabetes (Carter et al., 1997, Diabetic Med., 14:423-432; Gerstein et al., 1996, Diabetes Care, 19:1225-1228; Group, N. D. D. Diabetes in America, 1995, 2nd Edition; Haffner, 1998, Endocrine Reviews, 19:583-592; Lopes-Virella et al., 1996, Diabetes, 45:S40-44; Osanai et al., 1998, Am. J. Cardiol., 81:698-701; Stern, 1995, Diabetes, 44:369-374). Angioplasty, mechanical dilation of and obstructed blood vessel, is a common method for revascularization of the coronary and peripheral circulation. Restenosis, the obstruction of a blood vessel after mechanical dilation, is a serious complication and occurs more frequently in individuals with diabetes. This complication often necessitates surgical intervention or results in a myocardial infarction. In fact, the literature currently suggests that an individual with diabetes should undergo coronary artery bypass graft (CABG) surgery instead of angioplasty because of the high frequency of restenosis in diabetes (Influence of diabetes on 5-year mortality and morbidity in a randomized trial comparing CABG and PTCA in patients with multivessel disease:the Bypass Angioplasty Revascularization Investigation. Circulation 96: 1761-1769, 1997; Aikawa et al., Circulation 96:82-90, 1997; Anand-Srivastava et al., Mol. Cell. Biochem. 157:163-170, 1996; Bingley et al., J. Vasc. Surg. 28:308-318, 1998; Bonisch et al., Mol. Pharm. 54:241-248, 1998; Bourassa et al., J Am Coll Cardiol 33:1627-1636, 1999). There is great clinical morbidity as well as financial cost associated with the invasive alternative of CABG, therefore intensive study has focused on decreasing post-angioplasty restenosis.

Recent advances have greatly improved the understanding of vascular biology and the physiology of atherosclerosis and restenosis (Bornfeldt, Trends in Cardiovascular Medicine 6:143-151, 1996; Owens et al., J. Hypertension 14:S55-S64, 1996; Owens, Physiological Reviews 75:487-517,1995; Ross, Annu Rev Physiol 57:791-804, 1995). One aspect of vascular function that is altered in diabetes and restenosis is smooth muscle cell (SMC) phenotype. SMC undergo a change from a quiescent, contractile state to a proliferative, migratory status. This change is termed phenotypic modulation, and it is considered to be the hallmark of atherosclerosis and a key aspect of post-angioplasty restenosis. Vascular smooth muscle cell (SMC) migration and proliferation are central to the mechanism of post-angioplasty restenosis. Agents that decrease SMC proliferation have shown great promise in reducing restenosis.

The transition of arterial smooth muscle cells from a contractile to a synthetic, proliferative state appears to be an early event in the pathogenesis of atherosclerosis (Bornfeldt, Trends in Cardiovascular Medicine 6:143-151, 1996; Owens et al., J. Hypertension 14:S55-S64, 1996; Owens, Physiological Reviews 75:487-517, 1995; Ross, Annu Rev Physiol 57:791-804, 1995) and in angioplasty-induced restenosis (Absher et al., Atherosclerosis 143:245-251, 1999; Cook et al., Circ Res 74:189-196, 1994; Majack et al., J Cell Physiol 167:106-112, 1996; Mompeo et al., Ann Basc Surg 13:294-301, 1999). The vascular smooth muscle cell in mature animals is a highly specialized cell that proliferates at an extremely low rate and whose principal function is contraction. Mature SMC express specific proteins and signaling elements to serve this contractile state (Bornfeldt, Trends in Cardiovascular Medicine 6:143-151,1996; Owens et al.,J. Hypertension 14:S55-S64, 1996; Owens, Physiological Reviews 75:487-517, 1995; Ross, Annu Rev Physiol 57:791-804, 1995). Rapid and reversible changes in SMC phenotype occur in response to local environmental cues are important for normal blood vessel maintenance and vascular remodeling. In restenosis this remodeling process is exaggerated. As a result, SMC proliferate and migrate into the vessel lumen, which leads to obstruction. Numerous studies using SMC from diabetic animals and humans have demonstrated an increased migratory and proliferative phenotype (Absher et al., Atherosclerosis 143:245-251, 1999; Avena et al., J Nasc Surg 28:1038-1039, 1998; Kimura et al., Immunopharmacology 40:105-118, 1998; Wang et al., Nippon Ika Daigaku Zasshi 65:284-290,1998). Additionally, a recent study has demonstrated that the phenotypic modulation in primary SMC cultures from streptozotocin rats occurs more rapidly and more dramatically (Absher et al., Atheroscerosis 143:245-251, 1999; Avena et al., J Nasc Surg 28:1038-1039,1998; Etienne et al., Differentiation 63:225-236, 1998; Mompeo et al., Ann Basc Surg 13:294-301, 1999). Prior to the present invention, however, the mechanism behind the accelerated phenotypic modulation in diabetes remained unclear.

Angioplasty is a mechanical injury to the vessel wall, which denudes the endothelial lining and exposes SMC to circulating blood flow. SMC lose the tonic growth inhibition provided by endothelial cells and undergo phenotypic modulation. This response allows repair of the injury. Restenosis occurs when excessive SMC proliferation and migration results in large neointimal formation that occludes the vessel lumen. In contrast to the complex picture seen in atherosclerotic plaque, SMC make up a major portion of the neointimal thickening seen in post-angioplasty restenosis. The neointimal SMC are migratory, proliferative and resistant to apoptosis (Absher et al.,Atheroscerosis 143:245-251, 1999; Cook et al., Circ Res 74:189-196, 1994; Majack et al., J Cell Physiol 167:106-112, 1996; Mompeo et al., Ann Basc Surg 13:294-301, 1999). Restenosis is the major limitation to successful long-term coronary angioplasty and SMC migration and proliferation drive this process.

Therefore, there is a need to develop therapeutic protocols for the treatment of obesity, diabetes, cardiovascular disease, and macrovascular disease, as well as other diseases and conditions where dysregulation of cellular differentiation and/or an undesirable cell phenotype is problematic.

### SUMMARY OF THE INVENTION

One embodiment of the present invention relates to a method to modulate the phenotype of a target cell population in a patient. This method includes the step of administering to a patient a composition comprising a recombinant nucleic acid molecule comprising a nucleic acid sequence encoding a cyclic-AMP responsive element binding (CREB) protein having CREB biological activity operatively linked to a transcription control sequence. The CREB protein is expressed by the recombinant nucleic acid molecule in target cells in the patient. The target cells are preferably selected from the group of: (a) cells deficient in endogenous CREB expression; (b) cells deficient in endogenous CREB biological activity; and/or, (c) cells having normal endogenous CREB expression and biological activity which are predisposed to become deficient in endogenous CREB expression or biological activity. Expression of the CREB protein in the target cells is sufficient to modulate the phenotype of the cells.

The CREB protein can be any CREB protein as defined herein and in one embodiment, the CREB protein includes (a) a CREB protein having wild-type CREB biological activity; and/or, (b) a CREB protein having constitutively active CREB biological activity. Such CREB proteins include, but are not limited to, wild-type CREB protein, ATF-1, VP16-CREB, and/or CREB DIEDML. In one embodiment, the CREB protein is encoded by a nucleic acid sequence that hybridizes under stringent hybridization condition to a nucleic acid sequence selected from SEQ ID NO:1, SEQ ID NO:3, SEQ ID NO:5 and/or SEQ ID NO:20. In another embodiment, the CREB protein includes an amino acid sequence selected from SEQ ID NO:2, SEQ ID NO:4, SEQ ID NO:6, SEQ ID NO:21, an amino acid sequence comprising a biologically active fragment of SEQ ID NO:2, an amino acid sequence comprising a biologically active fragment of SEQ ID NO:4, an amino acid sequence comprising a biologically active fragment of SEQ ID NO:6, and/or an amino acid sequence comprising a biologically active fragment of SEQ ID NO:21. In another embodiment, the CREB protein is encoded by a nucleic acid sequence selected from the group of SEQ ID NO:1, SEQ ID NO:3, SEQ ID NO:5, SEQ ID NO:20, a fragment of SEQ ID NO:1 encoding a biologically active CREB protein, a fragment of SEQ ID NO:3 encoding a biologically active CREB protein, a fragment of SEQ ID NO:5 encoding a biologically active CREB protein, and/or a fragment of SEQ ID NO:20 encoding a biologically active CREB protein. In one embodiment, the transcription control sequence comprises a target cell-specific promoter, and in another embodiment, the promoter is inducible.

In one embodiment, the recombinant nucleic acid molecule comprises a viral vector. Such a viral vector can be from a virus which includes, but is not limited to alphaviruses, poxviruses, adenoviruses, herpesviruses, lentiviruses, adeno-associated viruses or retroviruses, with adenoviruses being a preferred embodiment.

In another embodiment, the composition further comprises a liposome that delivers the recombinant nucleic acid sequence into the cell. The liposome delivery vehicle can include lipids selected from the group of small unilamellar vesicle lipids, multilamellar vesicle lipids and/or extruded lipids. In one embodiment, the liposome delivery vehicle comprises cationic liposomes. In another embodiment, the liposome delivery vehicle comprises lipids selected from the group of DOTMA, DOTAP, DOTIM, DDAB and/or cholesterol. In yet another embodiment, the liposome delivery vehicle comprises a targeting agent that specifically binds to a molecule on the surface of the target cells. The targeting agent can include, for example, an antibody, a soluble receptor, and/or a ligand.

In yet another embodiment, the recombinant nucleic acid molecule is administered to the patient as naked DNA.

Preferably, the composition is administered by a route of administration selected from the group of ex vivo delivery, intravenous administration, intraperitoneal administration, intramuscular administration, intracoronary administration, intraarterial administration, subcutaneous administration, transdermal delivery, intratracheal administration, subcutaneous administration, intraarticular administration, intraventricular administration, inhalation, intracerebral, nasal, oral, pulmonary administration, impregnation of a catheter, and/or direct injection into a tissue. It is preferred that the CREB protein be expressed in the cell at a level of at least about 10,000 molecules of CREB protein per cell, and more preferably, at least about 25,000 molecules of CREB protein per cell, and even more preferably, at least about 50,000 molecules of CREB protein per cell. In one embodiment, the composition further comprises a pharmaceutically acceptable excipient.

In one embodiment of the present invention, the patient has or is at risk of developing diabetes. In this embodiment, the target cells are preferably selected from the group of adipocytes, vascular smooth muscle cells, cardiomyocytes, hepatocytes, skeletal muscle cells, beta cells, pituitary cells, synovial lining cells, ovarian cells, testicular cells, fibroblasts, endothelial cells, and/or neural cells. Preferably, the target cells have decreased CREB expression prior to the step of administering. Preferably, expression of the CREB protein in the target cells produces a result in the patient selected from: increased glucose control, decreased insulin resistance, reduced post-angioplasty restenosis, reduced atherosclerosis, reduced total body adiposity, normalization of lipid handling and normalization of hepatic glucose and/or protein handling.

In one embodiment, the target cells are adipocytes, and expression of the CREB protein results in a change in expression of a protein in the adipocytes selected from the group consisting of an increase phosphoenolpyruvate carboxykinase (PEPCK), an increase Glut4, an increase in PPAR γ, an increase in fatty acid synthetase (FAS), an increase in fatty acid binding protein (FABP), an increase in C/EBP α, an increase in CEBP β, an increase in LPL and/or a decrease in PREF-1. In another embodiment, the expression of the CREB protein in the adipocytes produces a result selected from the group consisting of decreased insulin resistance, normalized glucose control, and/or normalized lipid handling. In this embodiment, the step of administering is preferably by a route selected from the group consisting of intravenous administration, intraarterial administration, intraperitoneal administration and/or fat deposit injection. Preferred transcription control sequences include a promoter selected from the group consisting of aP2 promoter, lipoprotein L (LPL) promoter and/or leptin promoter.

In another embodiment, the target cells are vascular smooth muscle cells, and expression of the CREB protein modulates phenotypic characteristics in the vascular smooth muscle cells selected from the group of decreased proliferation of the cells, decreased migration of the cells, inhibition of cell cycle entry by the cells, increased contractility, decreased synthetic function and/or decreased cytokine expression. Preferably, expression of the CREB protein results in a change in expression of a protein in the vascular smooth muscle cell selected from the group consisting of a decrease in vascular endothelial growth factor (VEGF), an increase in endothelial nitric oxide synthase (eNOS), an increase in tissue-plasminogen activator (tPA), a decrease in plasminogen activator inhibitor-1 (PAI-1), a decrease in heparin binding-endothelial growth factor (HB-EGF), and/or a decrease in inducible nitric oxide synthase (iNOS). In one embodiment, expression of the CREB protein in the vascular smooth muscle cells produces a result in the patient selected from decreased susceptibility to post-angioplasty restenosis, reduced vessel occlusion, reduced atherosclerosis plaque formation, and/or decreased potential for pulmonary hypertension. In this embodiment, the transcription control sequence can include a promoter selected from α smooth muscle actin promoter and/or α smooth muscle myosin promoter. The step of administering is preferably by a route selected from the group consisting of intracoronary administration, intravenous administration, impregnation of angioplasty catheter, intraarterial administration, and/or pulmonary administration. In one embodiment, the step of administering is performed concurrent with or following angioplasty. In another embodiment, the patient has or is at risk of developing a condition selected from the group consisting of atherosclerosis, angina, acute myocardial infarction, stroke, pulmonary hypertension, amputation from peripheral vascular disease, and/or post-angioplasty restenosis.

In another embodiment of the invention, the patient has or is at risk of developing is heart failure. In this embodiment, the target cells are preferably cardiomyocytes, and expression of the CREB protein in the cardiomyocytes modulates phenotypic characteristics in the cardiomyocytes selected from the group consisting of expression of α-myosin heavy chain (α-MHC), spontaneous contraction, myocyte size, vacuolation and/or fibrosis. Preferably, expression of the CREB protein in the cardiomyocytes results in decreased characteristics associated with dilated cardiomyopathy in the patient. The transcription control sequence can include, for example, α-myosin heavy chain promoter, cardiac myosin light chain-2 promoter, β-myosin heavy chain promoter, cardiac troponin I promoter and/or cardiac troponin T promoter. In this embodiment, the step of administering is preferably by a route selected from the group consisting of intracoronary administration, intraventricular injection, intraarterial administration and/or intravenous administration.

In another embodiment of the present invention, the patient has or is at risk of developing pulmonary hypertension. In this embodiment, the target cells are preferably vascular smooth muscle cells.

In yet another embodiment, the patient has or is at risk of developing osteoarthritis, and the target cells are preferably synovial lining cells. In this embodiment, the step of administering is preferably by a route selected from the group consisting of injection into a joint where osteoarthritis is or may occur, intravenous administration, intraarticular administration, and/or intraarterial administration.

In another embodiment of the present invention, the target cells are neural cells. In this embodiment, the transcription control sequence can include, for example, a promoter selected from the group consisting of chromogranin A promoter, chromogranin B promoter Thy-1 promoter, and/or vgf promoter. A transcription control sequence can also include neuron restrictive enhancer elements. In this embodiment, the patient can have or be at risk of developing a disease or condition which includes, but is not limited to a spinal cord transsection, acute neuronal ischemia, Alzheimer's disease (wherein the preferred target cells are hippocampal neurons), Parkinson's disease, and/or depression (wherein the preferred target cells are cells of the cortex and basal ganglia). When the patient has Parkinson's disease, the preferred neural cells are dopaminergic neural transplant cells, and the step of administering comprises ex vivo delivery of the composition to the dopaminergic neural transplant cells, followed by transplantation of the dopaminergic neural transplant cells into the patient.

Another embodiment of the present invention relates to a method for restoring the ability of a cell to differentiate. The method includes the step of transfecting a cell deficient in CREB expression or CREB biological activity with a recombinant nucleic acid molecule comprising a nucleic acid sequence encoding a cyclic-AMP responsive element binding (CREB) protein having CREB biological activity, such that said CREB protein encoded by the recombinant nucleic acid molecule is expressed in the cell. Prior to the step of transfecting, the cell is not fully differentiated. Various aspects of this method are as described for the method to modulate phenotype described above.

Yet another embodiment of the present invention relates to a method to inhibit tumor neovascularization in a patient. This method includes the step of administering to said patient a composition comprising a recombinant nucleic acid molecule comprising a nucleic acid sequence encoding a cyclic-AMP responsive element binding (CREB) protein having dominant negative CREB biological activity operatively linked to a transcription control sequence. The CREB protein is expressed by said recombinant nucleic acid molecule in fibroblasts and endothelial cells in or near a tumor in said patient, and the expression of said CREB protein in said fibroblasts and endothelial cells is sufficient to modulate the phenotype of said fibroblasts and endothelial cells, resulting in inhibition of tumor neovascularization in said patient. The CREB protein having dominant negative CREB biological activity can include, but is not limited to KCREB, A-CREB, CREB M1, and/or a CREB DNA-binding region. In one embodiment, the CREB protein having dominant negative biological activity comprises an amino acid sequence selected from SEQ ID NO:6 and/or an amino acid sequence comprising a biologically active fragment of SEQ ID NO:6. In another embodiment, the nucleic acid sequence is selected from SEQ ID NO:5 and/or a fragment of SEQ ID NO:5 encoding a CREB protein having dominant negative CREB biological activity. Other aspects of this embodiment of the invention, including delivery vehicles, routes of administration, and levels of expression, have been described above with regard to CREB proteins having CREB biological activity. In a preferred embodiment, the composition is administered directly into said tumor.

Yet another embodiment of the present invention relates to a method to decrease total body adiposity. This method includes the step of administering to said patient a composition comprising a recombinant nucleic acid molecule comprising a nucleic acid sequence encoding a cyclic-AMP responsive element binding (CREB) protein having dominant negative CREB biological activity operatively linked to a transcription control sequence. The CREB protein is expressed by said recombinant nucleic acid molecule in adipocytes of said patient, and the expression of said CREB protein in said adipocytes is sufficient to inhibit differentiation of said adipocytes, resulting in a decrease in total body adiposity in said patient. Aspects of this embodiment of the invention have been described above, including delivery vehicles, routes of administration, and levels of expression, have been described above with regard to CREB proteins having CREB biological activity.

### BRIEF DESCRIPTION OF THE FIGURES

FIG. 1A is a digitized image of a Western analysis using antibodies specific for P-CREB or total CREB (CREB), showing that CREB is expressed in NIH 3T3-L1 preadipocytes.

FIG. 1B is a digitized image of a Western analysis using antibodies specific for P-CREB, total CREB (CREB), CEBPs α and β, and RXR α, showing that only CREB is expressed in NIH 3T3-L1 preadipocytes.

FIG. 1C is a bar graph showing that adipocyte differentiation-inducing agents stimulate CREB transcriptional activity in NIH 3T3 -L1 fibroblasts and adipocytes.

FIG. 2A is a line graph showing the time course of VP16-CREB expression in stably transfected NIH 3T3-L1 cells following muristerone induction.

FIG. 2B is a line graph showing the time course of KCREB expression in stably transfected NIH 3T3-L1 cells following muristerone induction.

FIG. 2C is a bar graph showing luciferase activity measured as an index of transcriptional activity in NIH 3T3-L1 clonal cell lines inducibly expressing VP16-CREB (clones 2-4 and 9-7) or KCREB (clones 2-1 and 2-10) and treated with 0.5 mM Bt2cAMP or 10 μM muristerone, or both agents together.

FIG. 3 is a digitized image showing that VP16-CREB stimulates and KCREB inhibits adipogenesis in 3T3-L1 cells.

FIG. 4A is an amino acid alignment of the promoter regions of several adipocyte-specific genes indicating the presence of putative CRE sequences.

FIG. 4B is a digitized image showing a representative autoradiogram of the free (bottom) and CREB bound complexes of twenty base pair, double stranded nucleotides and purified, recombinant 30 CREB protein in comparison to reactions performed with a non-specific (NS) oligonucleotide lacking a CRE sequence.

FIG. 4C is a digitized image showing nuclear extract protein prepared from 3T3-L1 fibroblasts incubated with the labeled oligonucleotides either in the absence (−) or presence (+) of CREB specific antibody.

FIG. 5 is a bar graph illustrating that CREB regulates transcription from adipocyte-specific gene promoters.

FIG. 6A is a digitized image showing that CREB content is decreased in mouse models of insulin resistance and diabetes.

FIG. 6B is a digitized image showing that CREB content is decreased in a rat model of diabetes.

FIG. 7 is a bar graph showing that treatment of BASMC with cAMP significantly attenuates cellular migration in response to PDGF.

FIG. 8A is a line graph showing BASMC stably transfected with muristerone-inducible CREB exposed to 10% serum in the absence of muristerone.

FIG. 8B is a line graph showing BASMC stably transfected with muristerone-inducible CREB exposed to 10% serum in the presence of muristerone.

FIG. 8A is a line graph showing BASMC stably transfected with muristerone-inducible CREB exposed to 0.1% serum in the absence of muristerone.

FIG. 8A is a line graph showing BASMC stably transfected with muristerone-inducible CREB exposed to 0.1% serum in the presence of muristerone.

FIG. 9A is digitized image and bar graph illustrating that ICER expression increased in a does dependent manner with increasing doses of VP16-CREB adenovirus.

FIG. 9B is a bar graph illustrating that infection of SMC with VP16-CREB adenovirus led to a significant increase in CREluc reporter gene activation.

FIG. 10A is a bar graph showing that introducing recombinant constitutively active CREB into SMC decreases mitogen-stimulated proliferation.

FIG. 10B is a bar graph showing that introducing recombinant constitutively active CREB into SMC decreases mitogen-stimulated migration.

FIG. 11 is a bar graph illustrating that VP-16 CREB adenovirus infection of SMC blunts cell migration induced by high glucose.

FIG. 12A is a digitized image showing that insulin increases PCREB/CREB content in BASMC.

FIG. 12B is a digitized image showing that insulin growth factor I (IGF-I) increases PCREB/CREB content in BASMC.

FIG. 12A is a digitized image showing that isoproterenol increases PCREB/CREB content in BASMC.

FIG. 13A is a bar graph showing that a wild-type CREB construct activated CREB-dependent Gal4-mediated luciferase production.

FIG. 13B is a bar graph showing that treatment of BASMC with PKA inhibitor H89 attenuates high endogenous CREB kinase activity.

FIG. 13C is a bar graph showing that treatment of BASMC with insulin increases CREB-dependent promoter activation.

FIG. 13D is a bar graph showing that ICER is a negative regulator of CREB activity.

FIG. 14 is a bar graph illustrating regulation of CREB dependent transcription by various CREB adenoviral constructs.

### DETAILED DESCRIPTION OF THE INVENTION

The present invention generally relates to a method to modulate cell phenotype by altering the expression and/or biological activity of cyclic AMP responsive element binding (CREB) protein in a target cell. Such a method is particularly useful in patients where dysregulation of cellular differentiation and/or development (or lack thereof) of a particular cell phenotype is, or is predicted to become, problematic. Such a method is also useful in conditions in which cellular differentiation may not be dysregulated, but in which the modulation of the phenotype is still desired for a therapeutic or cosmetic benefit to a patient. For example, as discussed above, diseases and conditions in which modulation of cell phenotype would be expected to provide a therapeutic benefit include, but are not limited to, obesity, diabetes, cardiovascular disease (e.g., congestive heart failure, cardiomyopathy), macrovascular disease (e.g., atherosclerosis, angina, acute myocardial infarction, stroke, pulmonary hypertension, and amputation from peripheral vascular disease), post-angioplasty restenosis, osteoarthritis, and neurodegenerative diseases and conditions of neural damage or dysregulation (e.g., Alzheimer's disease, Parkinson's disease, depression, acute ischemia and spinal transsection). Other diseases and conditions in which modulation of cell phenotype would be desirable will be apparent to those of skill in the art and are intended to be encompassed by the present invention.

The present inventors have discovered that the transcription factor CREB is necessary and sufficient to induce a modulation of cell phenotype, and in many cells, to induce cell differentiation. Moreover, the present inventors have discovered that CREB expression and/or activity is decreased or absent in a variety of cell types associated with various diseases and conditions, and that restoration of CREB content and/or activity to these cells is sufficient to initiate or increase a differentiation program or phenotypic modulation program in the cells such that the phenotype of the cells is modulated. Therefore, the present inventors have discovered that several diseases and conditions can be prevented and/or ameliorated simply by regulating the content of CREB in a cell. The present inventors have discovered that regulation of CREB content, in the absence of activating CREB in the cell or manipulating the cell in any other way is sufficient to modulate phenotype in the cell.

These discoveries are surprising, because it is unexpected that a single transcription factor among a large variety of transcription factors and other molecules involved in cell growth and differentiation would be both necessary and sufficient to modulate cell phenotype. Moreover, it is additionally surprising that simply by increasing the content of CREB in a cell, for example by introduction of a recombinant nucleic acid molecule encoding CREB, in the absence of activating the recombinant CREB or adding any other factor to the cell, is sufficient to modulate the phenotype of the cell. Prior to the present invention, it was known that CREB plays a role in gene transcription (i.e., for genes bearing the cAMP responsive element (CRE)) and is one of many cellular factors involved in a complex cell signaling network which is important for cell proliferation and differentiation. The vast majority of publications prior to the present invention regard CREB as a ubiquitous, constitutively expressed factor, which therefore would not be expected to participate in cell/tissue-specific processes of growth and differentiation. Such studies regard CREB as being a “target” for growth- and differentiation-related systems or one of many participants in these processes. Similarly, prior to the present invention, differentiation, or modulation of cell phenotype, was viewed as the result of multiple extracellular agents acting through numerous intracellular signaling systems and transcription factors, and not as process controlled by a single transcription factor. Indeed, it is believed that most experts in the field regard CREB as a regulator of metabolism and neuroendocrine activities, but not as a global participant in cell proliferation and differentiation. Although a few investigators have noted a potential role for CREB and related proteins in cell proliferation, differentiation and phenotypic modulation, it was not known until the discovery by the present inventors, that CREB is both necessary and sufficient to initiate cell differentiation/phenotypic modulation programs. The present inventors have also discovered that CREB deficiency, in content and/or in activity, is correlated with dysregulation of phenotypic modulation, including dysregulation of differentiation, in many cell types, including adipocytes and vascular smooth muscle cells, and that solely by increasing the content of CREB in such cells, phenotypic modulation can be increased, for example.

The present inventors have therefore provided a therapeutic protocol by which a disease or condition in which dysregulation of phenotypic modulation or more particularly, cell differentiation, is problematic or in which modulation of a particular cell phenotype would provide a therapeutic or cosmetic benefit, can be treated. The inventors' discovery is significant, because the treatment is carried out simply by increasing the CREB content in a target cell, in the absence of adding any other factors or manipulating the cell in any other way. Similarly, the present inventors have discovered that there are certain conditions under which a modulation of phenotype such as a decrease or inhibition of cell differentiation is desirable, and whereby the phenotype of a cell can be modulated in a targeted cell solely by inhibiting the action of CREB, without the need to affect other transcription factors or signal transduction molecules in the cell.

As discussed above, prior to the present invention, several groups had produced data which promoted a potential role for CREB and related factors in cell growth and differentiation. For example, one group showed that ectopic expression of a dominant negative CREB protein in pituitary somatotrophic cells leads to somatotroph hypoplasia and dwarfism in transgenic mice (Struthers et al., 1991, Nature 350:622-624). Similarly, ectopic expression of a dominant negative ATF1 protein (another transcription factor protein related to CREB) has been shown to block cAMP-induced neurite outgrowth in PC12 cells (Shimomura et al., 1998, J. Neurochem., 70:1029-1034), and targeted expression of a dominant negative CREB in cardiac myocytes has been shown to produce idiopathic-dilated cardiomyopathy with exaggerated heterogeneity in myocyte phenotype (Fentzke et al., 1998, J. Clin. Investigation, 101:2415-2426). Surface antigen receptor activation of B lymphocyte proliferation appears to involve enhanced CREB phosphorylation in response to elevated PKA and PKC activity and downregulation of PP2A (Amato et al., 1997, J. Immunol, 159:4676-4685; Xie et al., 1995, J. Immunol., 154:1717-1723; Xie et al., 1996, Cell Immunol., 169:264-270), and expression of dominant negative CREB in T lymphocytes blocks their proliferation following activation (Barton et al., 1996, Nature (London), 379:81-85). CREB null transgenic mice exhibit perinatal mortality, reduced corpus callosum and anterior commissures in the brain, decreased thymic cellularity, and impaired T lymphocyte development (Rudolph et al., 1998, Proc. Nat'l. Acad. Sci. USA, 95:4481-4486). Yarwood and colleagues showed that cAMP enhances growth hormone-dependent differentiation of preadipocytes and suggests that CREB may potentiate this activity (Yarwood et al., 1998, Mol. Cell. Endocrinol. 138:41-50). Cyclic-AMP signaling to CREM and ICER via PKA has been shown to play a role in hepatocyte proliferation (Servillo et al., 1997, Genes and Development, 12:3639-3643; Servillo et al., 1997, Oncogene 14:1601-1606), and CREB phosphorylation directly inhibits hepatic stellate cell proliferation (Houglum et al., 1997, J. Clin. Investigation, 99:l322-1328). Similarly, cAMP-induced ICER IIγ expression blocks the proliferation of either mouse pituitary tumor cells or human choriocarcinoma cells at the G2/M boundary (Razavi et al., 1998, Oncogene, 17:3015-3019). Lamas, et al. (Lamas et al., 1997, Mol. Endo., 11:1425-1434) have reported that ICER modulates pituitary corticotroph proliferation.

However, no prior investigator has demonstrated or suggested that CREB is both necessary and sufficient to induce a phenotypic modulation program (e.g., a differentiation program) in a cell, and it is completely unexpected that by simply increasing the content of a single transcription factor, CREB, within a cell, phenotypic modulation can be induced. This is significant because, prior to the present invention, the existing data would lead one of skill in the art to the conclusion that a complex interaction of several cellular factors was responsible for the initiation of phenotypic modulation and that induction of phenotypic modulation (e.g., induction of differentiation) would require the specific activation of these multiple factors. Moreover, it would be unexpected that introduction to a cell of an unactivated recombinant transcription factor would be capable of inducing phenotypic modulation in a cell in which a differentiation program was not otherwise activated. Indeed, although a few investigators have observed an effect on cell growth and/or development (Struthers et al., Shimomura et al., Rudolph et al.) or an altered cell phenotype (Fentzke et al.) when a dominant negative CREB or ATF1 protein was introduced into particular cell types in vitro, these investigators have failed to appreciate that CREB is necessary and sufficient for initiation of phenotypic modulation and that restoration of CREB expression and/or activity in cells which are deficient in CREB is sufficient to induce phenotypic modulation and particularly, to increase differentiation, in the cell. Finally, prior to the present invention, it was not known that inhibition of CREB in adipocytes, even in the presence of insulin, would be sufficient to dedifferentiate adipocytes, or that inhibition of CREB in vascular smooth muscle cells would be sufficient to decrease or prevent differentiation of vascular smooth muscle cells (i.e., inhibit contractile functions and slow growth and increase proliferative and invasive phenotype).

More particularly, in one aspect of the present invention, the present inventors have demonstrated that CREB is necessary and sufficient to initiate the adipocyte differentiation program. This conclusion is based in part on the constitutive expression of CREB in 3T3-L1 fibroblasts prior to the induction of adipogenesis and throughout the differentiation process, as demonstrated by the present inventors and discussed in detail in the Examples section. Furthermore, both CREB phosphorylation and transcriptional activity are rapidly induced in 3T3-L1 fibroblasts by conventional differentiation-inducing agents, and CREB has been shown to bind to and stimulate transcription from the promoters of several adipocyte-specific genes. Most importantly, the present inventors have directly demonstrated that CREB stimulates adipogenesis through their ability to induce adipocyte differentiation with constitutively active, VP16-CREB, and to completely block the efficacy of normal differentiation-inducing agents with dominant negative KCREB.

Prior to the present invention, research had indicated that CREB might be one of the intracellular molecules which played a role in adipogenesis. Previous reports showed that both CREB phosphorylation and transcriptional activity were stimulated by agents that induce adipocyte differentiation such as Bt₂cAMP acting through the cAMP-dependent protein kinase (PKA), and insulin via an ERK ½ kinase cascade (Klemm et al., 1998, J. Biol. Chem., 273:917-923) and decreased nuclear protein phosphatase 2A activity (Reusch et al., 1995, Endocrinology, 136:2464-2469; Reusch et al., 1994, Endocrinology, 135:2418-2422). A number of groups have demonstrated similar increases in CREB phosphorylation and activity in response to other growth factors including Nerve Growth Factor (NGF) and Fibroblast Growth Factor (FGF) via ERK ½ and p38 Mitogen Activated Protein (MAP) Kinase pathways, respectively (Ginty et al., 1994, Cell, 77:713-725; Tan et al., 1996, EMBO J., 15:4629-4642). CREB is also regulated by several viral proteins, some of which alter cellular growth and differentiation Adya et al., 1994, Proc. Natl. Acad. Sci. USA, 91:5642-5646; Arany et al., 1995, Nature, 374:81-84; Barnabas et al., 1997, J. Biol. Chem., 272:20684-20690; Giebler et al., 1997, Mol. Cell. Biol., 17:5156-5164; Lee et al., 1996, J. Biol. Chem., 271:17666-17674; Lundblad et al., 1995, Nature, 374:85-88; Maguire et al., 1991, Science, 252:842-844; Wheat et al., 1994, Mol. Cell. Biol. 14:5881-5890) and other members of the CREB/Activating Transcription Factor (ATF) family of transcription factors that bind the same cis-acting promoter sequences as CREB are targets for various growth factor signaling systems and viral transforming proteins (Abdel-Hafiz, et al., 1992, Mol Endo. 6:2079-2089; Chatton et al., 1993, Mol. Cell. Biol., 13:561-570; Gupta et al., 1995, Science, 267:389-393; Liu et al., 1990, Cell, 61:1217- 1224; Maguire et al., 1991, Science, 252:842-844; Tan et al., 1996, EMBO J., 15:4629-4642). Although these data indicate that CREB is potentially involved in the regulation of cell proliferation and differentiation, it was not until the present inventors' surprising discovery that it was known that CREB is necessary and sufficient for initiation of adipocyte differentiation.

The present inventors' showing of the induction of adipogenesis by VP16-CREB alone indicates that CREB activation is sufficient to induce this process, whereas the ability of KCREB to block adipogenesis indicates that CREB is a necessary participant in adipocyte development. These conclusions are significant, because factors previously identified as participants in adipogenesis have now been shown by the present inventors to only be expressed in significant levels following initiation of the differentiation program. Without the discovery of the present inventors that increasing CREB content alone induces adipogenesis, it might have been believed that CREB was only one of many necessary participants in development of the adipocyte, as other investigators have concluded with regard to the role of CREB in a variety of cell types. However, the present inventors' results led to the discovery that CREB is a sole inducer of adipogenesis, and therefore, a target for intercellular signaling mechanisms that recruit the development of new fat cells in hyperplastic obesity. Furthermore, CREB and the signaling systems that impinge on CREB are disclosed herein to be targets for therapeutic agents to treat or prevent obesity. Interestingly, preliminary experiments in the present inventors' laboratory indicate that constitutive overexpression of KCREB in mature adipocytes leads to their dedifferentiation with loss of triacylglycerol vesicles, even in the presence of insulin (data not shown). Unger and colleagues (Zhou et al., 1999) have recently reported a similar reversal of adipocyte phenotype in normal rats following overexpression of leptin. These studies support the contention that adipocyte development and function can be regulated at various levels, opening the door to novel strategies designed to address obesity and related disorders. The present inventors have opened a new door to such strategies through the modulation of CREB.

In another aspect of the present invention, the present inventors have demonstrated that CREB is necessary and sufficient for phenotypic modulation of vascular smooth muscle cells. Smooth muscle cells isolated from discrete compartments of the mature vascular media exhibit unique phenotypes and distinct growth capabilities. Dempsey and Stenmark have defined four unique SMC populations in the inner, middle and outer compartments of the arterial media (Frid et al., Circ Res 81:940-952, 1997; Frid et al., Arterioscler Thromb Vasc Biol 17:1203-1209, 1997; Stenmark et al., Chest 114:82S-90S, 1998). These cells demonstrate differences in morphological appearance, expression of muscle specific proteins and growth capabilities. The L2 and L3c cells exhibit characteristics of well-differentiated SMC with contractile function and slow growth (i.e., are proliferation-resistant); whereas, the L1 and L3I cells grow rapidly in culture (i.e., are proliferation prone) and exhibit non-muscle cell characteristics. The present inventors have demonstrated that the well-differentiated, proliferation-resistant subpopulations have increased CREB protein content as compared to the less-differentiated, proliferation prone subpopulations. In addition, the present inventors have demonstrated a decrease in arterial blood vessel wall content of CREB in diabetes and insulin resistance, that induction of CREB expression in vascular smooth muscle cells inhibits SMC migration, proliferation and entry into cell cycle, and that high glucose changes SMC phenotype (migration, proliferation, and synthetic function) which can be restored to normal by the expression of CREB. Therefore, it is an embodiment of the present invention to modulate the phenotype of vascular smooth muscle cells toward a more well differentiated SMC phenotype exhibited by L2 and L3c cells by increasing the expression of CREB in the cells, or alternatively, to modulate the phenotype of vascular smooth muscle cells toward a less differentiated SMC phenotype exhibited by L1 and L3I cells by inhibiting CREB expression and/or activity in the cells.

It has been hypothesized that CREB is important for protection from toxins and mechanical injury (Walton et al., 1992, Mol. Endo., 6:647-655). Indeed melanoma cells are more sensitive to radiation injury when a dominant negative CREB is overexpressed, suggesting that CREB is protective against UV induced apoptosis in these cells (Yang et al., 1996, Oncogene, 12:2223-2233). Neurotoxic states such as acute ethanol exposure, formalin induced nerve injury, hyperglycemia and hypoxia induce CREB phosphorylation in neuronal tissues (Beitner-Johnson et al., 1998, J. Biol. Chem., 273:19834-9; Deisseroth et al., 1996, Neuron, 16:89-101; Hermanson et al., 1997, Brain Res. Mol. Brain Res., 51:188-96; Kvietikova et al., 1997, Kidney Int., 51:564-6; Salminen et al., 1998, Brain Res. Mol. Brain Res., 61:203-206; Schulman, 1994, Curr. Opin. Neurobiol., 5:375-81; Yang et al., 1998, Alcohol Clin. Exp. Res., 22:382-90; Yoshida et al., 1998, J. Biol. Chem., 273:33741-3 3749). Chronic ethanol exposure decreases this acute CREB response (Yang et al., 1998, Alcohol Clin. Exp. Res., 22:382-90). CREB content and DNA binding is decreased in senescent cells (Chin et al., 1996, Am. J. Physiol., 271:C362-C371). In endothelial cells, CREB binding to the fibronectin promoter decreases with age (Kumazaki et al., 1996, Mech. Aging Dev., 88:111-124). Depression decreases CNS CREB content and it is restored by antidepressants (Duman, 1998, Biol. Psychiatry, 44:324-35). These studies suggest that chronic toxin exposure may exhaust the acute CREB response and decrease CREB content, and in view of the present inventors' discovery that CREB is necessary and sufficient to initiate the modulation of phenotype in a cell, conditions in which chronic toxin exposure has occurred are also suitable conditions for use of the method of the present invention.

One embodiment of the present invention is a method to modulate the phenotype of a target cell population in a patient. This method includes the step of administering to a patient a composition comprising a recombinant nucleic acid molecule comprising a nucleic acid sequence encoding a cyclic-AMP responsive element binding (CREB) protein having CREB biological activity operatively linked to a transcription control sequence. The administration of the recombinant nucleic acid molecule results in the expression of the recombinant CREB protein by the recombinant nucleic acid molecule in target cells in the patient. The target cells are selected from the group of: (a) cells deficient in endogenous CREB expression; (b) cells deficient in endogenous CREB biological activity; and, (c) cells having normal endogenous CREB expression and biological activity, which are predisposed to become deficient in endogenous CREB expression or biological activity. Expression of the CREB protein in the target cells is sufficient to modulate the phenotype of the cells.

According to the present invention, the “phenotype” of a cell is any observable characteristic of a cell, and is effectively the functional expression of the genetic information (gene activity) in a given cell. The term “modulate” or derivatives of such term, means to change, regulate or vary from one state to another, and includes a measurable or observable increase or decrease in any measurable characteristic and/or a change from one characteristic to another, different characteristic. Therefore, the phrases “modulate the phenotype” or “phenotypic modulation” refer to any measurable or observable change in any measurable or observable characteristic of a cell. As such, a phenotypic modulation can be any measurable change, for example, in the morphology of a cell, in the expression of one more proteins by a cell, in the functional characteristics of a cell (e.g., contractility, migratory behavior, secretion of a particular factor), and/or in the growth factor requirements of the cell. As used herein, cellular differentiation is a type (i.e., a subset) of phenotypic modulation, and the phrase “differentiation” describes one or more cellular processes by which a cell undergoes a change (i.e., a phenotypic modulation) to an overtly specialized or defined cell type. Therefore, the term “phenotypic modulation” can be used to refer to “differentiation” of a cell to the extent that differentiation is described herein as a type of phenotypic modulation. However, for purposes of clarity, the term “differentiation” is not necessarily used interchangeably with the term “phenotypic modulation,” but rather, differentiation is considered herein to be a subset of phenotypic modulation. More specifically, for a given cell type which has exhibited a phenotypic modulation, such phenotypic modulation may not be considered in the art to be indicative of initiation of a differentiation program in the cell. Instead, the particular phenotypic modulation may be considered to be functional phenotypic change within a single differentiated phenotype. One type of phenotypic modulation is entry into or exit from cell cycle, although generally, the actual process of proliferation, or cell division, by a cell of stable phenotype is not considered to be a phenotypic modulation of a cell, since the cell does not undergo a change in an observable characteristic while it is dividing.

Wild-type, or naturally occurring, CREB is a 43-kDa nuclear transcription factor that is constitutively expressed in most cells and tissues (Gonzales et al., 1989, Cell, 59:675-680; Gonzalez et al., 1989, Nature, 337:749-752; Hoeffler et al., 1990, Trends in Endocrinology & Metabolism, 1:155-158; Lamph et al., 1990, Proc. Natl. Acad. Sci. USA, 87:4320-4324; Lee et al., 1990, EMBO J., 9:4455-4465; Sassone-Corsi et al., 1998, Genes Dev., 2:1529-1538; Sun et al., 1992, Mol. Endo., 6:1858-1866; Yamamoto et al., 1988, Nature, 334:494-498). It was originally identified in the regulation of gene transcription in response to elevated, intracellular cAMP levels in tissues. In this capacity, CREB binds to a specific target sequence or cAMP-response element (CRE), the consensus sequence of which is represented herein as (5′-TGACGTCA-3′) SEQ ID NO:9, in the promoter regions of cAMP-regulated genes. Under conditions that increase intracellular cAMP levels, CREB is phosphorylated by the catalytic subunit of cAMP-dependent protein kinase (PKA) at serine 133. Phosphorylation of CREB (PCREB) increases its association with transcriptional adapter proteins like CREB Binding Protein (CBP) or P300 which interact with the basal transcriptional machinery and thereby increase the rate of transcription. CREB's serine 133 phosphorylation state is determined by the level of activity of numerous intracellular signaling cascades in addition to PKA, such as, ERK ½ MAP kinase, p38 MAP kinase, PKC, and PI-3 kinase in response to multiple growth factors, calcium, immunoglobulins (IgG) and oxidant stress. Multiple CREB kinases activated by these upstream signaling pathways have been identified, including PKA; RSK 1,2,3; MAPKAP kinase 2 and 3; MSKI and 2; and calmodulin kinase. Transient phosphorylation and activation of CREB is the classic PCREB response. CREB dephosphorylation by phosphatases PP-1 and PP2A also appears to be crucial for CREB regulation. Phosphorylation of CREB on serine 133, which is one determinant of CREB transcriptional activity (CREB-TA), can be viewed as a nuclear read out of the convergence of multiple signaling pathways.

According to the present invention, a “CREB protein having CREB biological activity” can be a full-length CREB protein or any homologue of such a protein, including a protein in which amino acids have been deleted (e.g., a truncated version of the protein, such as a biologically active peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). A homologue of a CREB protein is a protein having an amino acid sequence that is sufficiently similar to a naturally occurring CREB protein amino acid sequence that the homologue has substantially the same or enhanced biological activity compared to the corresponding naturally occurring protein. The functional domains of a wild-type CREB protein are known in the art, and therefore, one of skill in the art would be able to selectively modify a wild-type CREB protein as discussed above to develop a CREB homologue with CREB biological activity. For example, it is known that the leucine zipper domain of the CREB protein in the C-terminal portion is important for DNA binding (from about positions 308-341 of rat CREB (SEQ ID NO:4) and from about positions 294 to 327 of human CREB (SEQ ID NO:2)). The basic region of the C-terminal portion of the CREB protein is important for wrapping around the DNA and therefore is also important for binding (from about positions 274-308 of rat CREB (SEQ ID NO:4) and from about positions 260 to 294 of human CREB (SEQ ID NO:2)). In the N-terminal portion of CREB, the kinase inducible domain is important for the activation and biological activity of wild-type CREB (from about positions 101 to 160 of rat CREB (SEQ ID NO:4) and from about positions 101-147 of human CREB (SEQ ID NO:2)). Interspersed throughout the N-terminal region of wild-type CREB (i.e., called the transactivation domain and including the kinase inducible domain) are several acidic amino acid residues which are believed to potentiate CREB biological activity.

In one embodiment, a homologue of a CREB protein has CREB biological activity and is encoded by a nucleic acid molecule which hybridizes under stringent hybridization conditions to a nucleic acid sequence encoding a naturally occurring CREB protein. Nucleic acid sequences encoding naturally occurring CREB proteins are known in the art and include the nucleic acid sequence encoding human CREB, represented herein as SEQ ID NO:1, and the nucleic acid sequence encoding rat CREB, represented herein as SEQ ID NO:3.

As used herein, stringent hybridization conditions refer to standard hybridization conditions under which nucleic acid molecules are used to identify similar nucleic acid molecules. Such standard conditions are disclosed, for example, in Sambrook et al., Molecular Cloning A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138,267-284; Meinkoth et al., ibid., is incorporated by reference herein in its entirety.

More particularly, stringent hybridization and washing conditions, as referred to herein, refer to conditions which permit isolation of nucleic acid molecules having at least about 75% nucleic acid sequence identity with the nucleic acid molecule being used to probe in the hybridization reaction, more particularly at least about 80%, and more particularly at least about 85%, and most particularly at least about 90%. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C., more preferably, between about 28° C. and about 40° C., and even more preferably, between about 35° C. and about 45° C. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 TO 9.62.

Homologues of naturally occurring CREB proteins are known in the art. For example, several constitutively active CREB proteins are described in detail herein and are considered to be CREB protein homologues. Additionally, a protein known as activating transcription factor-1 (ATF1) is considered to be a CREB protein homologue according to the present invention. ATF-1 is structurally homologous to a wild-type, or naturally occurring CREB protein (i.e., human ATF-1 is approximately 75% homologous to human CREB at the amino acid level), in that the amino acid sequence of ATF-1 shares high identity with CREB protein amino acid sequence throughout the length of ATF-1, with ATF-1 lacking the N-terminal 94 amino acid residues of CREB protein (i.e., a truncation within the transactivation domain, as compared to CREB). The nucleic acid sequence for human ATF-1 is represented herein by SEQ ID NO:20. SEQ ID NO:20 encodes an amino acid sequence represented herein as SEQ ID NO:21. ATF-1 can form a heterodimer with CREB and has CREB biological activity as discussed below.

As used herein, the biological activity or biological action of a protein refers to any function(s) exhibited or performed by a naturally occurring form of the protein as measured or observed in vivo (i.e., in the natural physiological environment of the protein) or in vitro (i.e., under laboratory conditions). For example, a biological activity of a wild-type CREB protein can include, but is not limited to, activation of the protein (e.g., phosphorylation of the protein, upregulated expression of the protein), protein binding activity (e.g., with CBP), protein translocation, DNA binding activity (i.e., with CRE sequences), induction of transcription, and/or initiation of phenotypic modulation in a cell. Modifications of a protein, such as in a homologue or mimetic (discussed below), which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

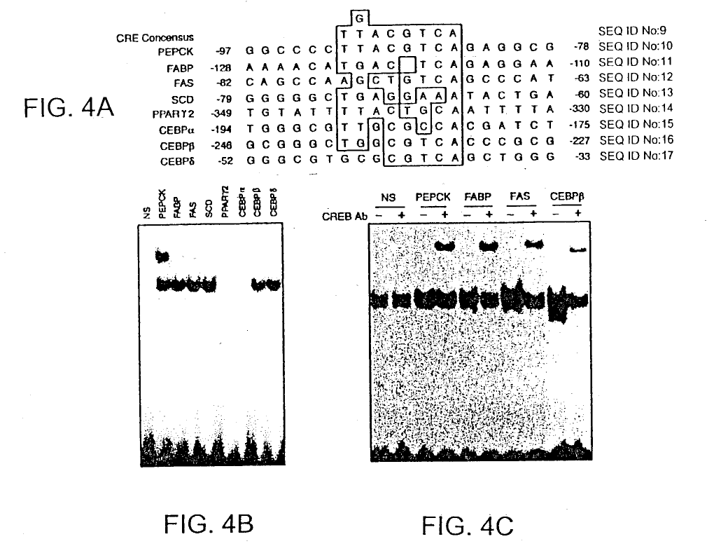

As used herein, a protein that has “CREB biological activity” refers to a protein (as described above) that is capable of modulating (i.e., increasing or decreasing) the activation of cAMP-response element (CRE)-dependent transcription. In other words, such a protein can modulate the transcriptional activity of a gene which has a CRE sequence in its regulatory region. The consensus sequence for CRE is represented-herein by SEQ ID NO:9, although multiple variants of the consensus sequence are known in the art. A number of CRE sequences are shown, for example, in FIG. 4A. CRE sequences can also be determined using a publicly available transcription element search system (TESS; Computational Biology and Infomatics Laboratory, School of Medicine, University of Pennsylvania). Preferably, a protein that has CREB biological activity can bind to a specific target DNA sequence or cAMP-response element. In one embodiment, a protein that has CREB biological activity is characterized by an ability to associate, when activated, with transcriptional adapter proteins like CREB Binding Protein (CBP) or P300 in a manner which results in an increase in transcription of a gene containing a CRE sequence. Such an association is a characteristic of a wild-type CREB protein, for example, although it is not necessarily a characteristic of a CREB homologue, such as a constitutively active CREB protein or a dominant negative CREB protein (discussed below). To become activated, a wild-type CREB protein that has CREB biological activity is typically phosphorylated on an amino acid residue which is equivalent to serine 133 in the naturally occurring protein, although such a characteristic is not necessarily a characteristic of a CREB homologue, such as a constitutively active CREB protein or a dominant negative CREB protein. If the protein that has CREB biological activity is a fragment of the naturally occurring protein, the fragment contains at least the minimum portion of the full-length CREB necessary for CREB biological activity, which includes serine 133 of the naturally occurring, full-length protein. In preferred embodiment, a protein that has CREB biological activity has an amino acid sequence which is substantially similar to the transactivation domain of the amino acid sequence of a naturally occurring CREB protein such that the protein can associate with transcriptional adapter proteins in a manner which results in an increase in transcription of a gene containing a CRE sequence. In a more preferred embodiment, a protein that has CREB biological activity has an amino acid sequences that are substantially similar to the transactivation domain, and the DNA binding domain of a naturally occurring CREB protein such that the protein can associate with transcriptional adapter proteins in a manner which results in an increase in transcription of a gene containing a CRE sequence and can bind to a specific target DNA sequence or cAMP-response element (CRE), for example.

CREB biological activity can be evaluated by one of skill in the art by any suitable in vitro or in vivo assay. Such assays include, for example, determining the expression of CREB protein or CREB transcriptional activity in a cell (e.g., by Western or Northern blot), determining changes in phosphorylation of CREB (wild-type) in a cell, and determining the modulation of cell phenotype in a cell by performing differentiation assays, immunohistochemistry, cell proliferation assays, and morphological assays, for example, using recombinant CREB proteins, inducible promoters, and measurable, markers in such assays. More specifically, assays for CREB biological activity include, but are not limited to, the following types of assays: (1) CREB transcriptional activity assays (transient and stable transfection assays with CREB responsive promoters linked to reporter genes (e.g., luciferase, CAT); nuclear run-on transcription assays; transfection assays with Gal4 responsive promoters/reporter genes and chimeric Gal4-CREB proteins; (2) CREB content and phosphorylation assays (Western blots, northern blots, immunohistochemistry); (3) CREB functional assays (proliferation assays, differentiation assays). Several assays for CREB biological activity in different cell types are described in detail in the Examples section, and additional assays and methods are described in detail, for example, in Klemm et al., 1998, J. Biol. Chem., 273:917-923; Reusch et al., 1995, Endocrinology, 136:2464-2469; Reusch et al., 1994, Endocrinology, 135:2418-2422; and Pugazhenthi et al., 1999, J. Biol. Chem., 274:2829-2837; all of which are incorporated herein by reference in their entirety.

Preferred proteins having CREB biological activity which can be expressed in a cell according to the method of the present invention include, but are not limited to any isolated and/or recombinantly produced wild-type (e.g., naturally occurring) CREB protein and any constitutively active CREB protein. According to the present invention, a wild-type CREB protein is a CREB protein that can be isolated from any species of the kingdom, Animalia, and which is characterized by its ability to bind to a specific target sequence or cAMP-response element (CRE), which is activated by transient phosphorylation, and which, when activated, associates with transcriptional adapter proteins in a manner which results in an increase in transcription of a gene containing a CRE sequence. A wild-type CREB protein can include isolated proteins encoded by CREB genes and by naturally occurring allelic variants of CREB genes. According to the present invention, a CREB gene encodes a cyclic AMP (cAMP) responsive element binding (CREB) protein and includes all nucleic acid sequences related to a natural CREB gene such as regulatory regions that control production of the CREB protein encoded by that gene (such as, but not limited to, transcription, translation or post-translation control regions) as well as the coding region itself. As used herein, an allelic variant of a CREB gene is a gene that occurs at essentially the same locus (or loci) in the genome as the CREB gene, but which, due to natural variations caused by, for example, mutation or recombination, has a similar but not identical nucleic acid sequence. Allelic variants typically encode proteins having similar activity to that of the protein encoded by the gene to which they are being compared. Allelic variants can also comprise alterations in the 5′ or 3′ untranslated regions of the gene (e.g., in regulatory control regions). Allelic variants are well known to those skilled in the art and would be expected to be found within a given species of the kingdom, Animalia, and particularly within Homo sapiens.

A CREB protein can also include a fusion protein, that includes a CREB protein-containing domain attached to one or more fusion segments. Suitable fusion segments for use with the present invention include, but are not limited to, any segments that can enhance the biological activity of the CREB protein or enhance the CREB protein's stability in the host cell. A suitable fusion segment can be a domain of any size that has the desired function.

Particularly preferred wild-type CREB proteins include human and rat CREB proteins. CREB proteins are highly conserved among animal species, and particularly, between species of the vertebrate class, Mammalia: By way of example, the human and rat CREB proteins are about 96% homologous at the nucleic acid level in the coding region, and about 99% homologous at the amino acid level (Hoeffler et al., 1990, Mol. Endo., 4:920-930; Hai et al., 1989, Genes and Develop. 3:2083-2090). Therefore, a CREB protein from one species of animal is biologically active in a cell from a different species of animal, particularly with regard to mammals. For example, the present inventors have expressed biologically active recombinant CREB proteins from human or rat in bovine, rat, mouse and human cells.

The nucleic acid and amino acid sequences of at least human and rat CREB proteins are known in the art. For example, the coding region of a nucleic acid molecule encoding a human CREB protein is represented herein as SEQ ID NO:1. SEQ ID NO:1 encodes a CREB protein having an amino acid sequence represented herein as SEQ ID NO:2. The coding region of a nucleic acid molecule encoding a rat CREB protein is represented herein as SEQ ID NO:3. SEQ ID NO:3 encodes a CREB protein having an amino acid sequence represented herein as SEQ ID NO:4. The coding region of a nucleic acid molecule encoding a human ATF-1 protein is represented herein as SEQ ID NO:20. SEQ ID NO:20 encodes an ATF-1 protein having an amino acid sequence represented herein as SEQ ID NO:21. It is to be understood that a nucleic acid sequence encoding the amino acid sequences identified herein can vary due to degeneracies. The nucleic acid and amino acid sequences of CREB proteins for at least a bovine species, Bos Taurus (Genbank Accession No. AF006042), can be found in public sequence databases, for example, Genbank. Therefore, proteins having an amino acid sequence of SEQ ID NO:2, SEQ ID NO:4, SEQ ID NO:21 and homologues thereof (e.g., SEQ ID NO:6, described below), are also preferred CREB proteins for use in the present invention. As used herein, nucleotide degeneracies refers to the phenomenon that one amino acid can be encoded by different nucleotide codons.

According to the present invention, a CREB protein having constitutively active CREB biological activity is a CREB protein which has been modified from the corresponding naturally occurring CREB protein, such that resulting protein is continuously activated. A constitutively active CREB can be modified, for example, by insertion, deletion, substitution or derivatization of the amino acids from the naturally occurring, or wild-type, CREB sequence, such that the modified CREB protein is constitutively active. Such modifications can include, but are not limited to, modifications which remove the requirement for phosphorylation for activation while leaving the protein in an activated state, modifications which maintain the active, phosphorylated state, or modifications which increase the binding of the transcriptional adapter proteins, such as CREB binding protein (CBP). Other modifications which result in constitutively active CREB will be apparent to those of skill in the art. Constitutively active CREB proteins are known in the art. By way of example, a constitutively active CREB protein useful in the present invention is described in the Examples section and is referred to as VP16-CREB. Constitutively active VP16-CREB is a fusion protein comprising the transactivation domain of the viral VP16 protein (amino acids 412-490 of the VP16 protein) linked to the CREB DNA binding domain (amino acids 217-327), the resulting protein of which is represented herein by SEQ ID NO:6. SEQ ID NO:6 is encoded by SEQ ID NO:5. Another constitutively active CREB protein is referred to herein as CREB DIEDML. CREB DIEDML is a full length CREB with a series of mutations that leads to constitutive binding to CREB binding protein (CBP). CBP interacts with the transcriptional machinery to activate CRE dependent transcription. CREB DIEDML is described in the Examples section.

In certain embodiments of the method of the present invention, which are discussed in detail below, it is desirable to inhibit the expression and/or biological activity of the endogenous CREB expressed by a cell. In these embodiments of the present invention, a recombinant nucleic acid molecule including a nucleic acid molecule encoding a protein having dominant negative CREB biological activity is employed. According to the present invention, a protein having dominant negative CREB biological activity, also referred to as dominant negative CREB, is a CREB protein which has been modified from the corresponding naturally occurring CREB protein, such that the resulting protein has a biological activity that inhibits the biological activity of a wild-type or endogenous CREB protein. For example, a dominant negative CREB protein can inhibit the biological activity of a wild-type CREB protein by: binding to the endogenous CREB so that the endogenous CREB is prevented from binding to other proteins; by competing against the endogenous CREB for binding substrates (i.e., DNA or protein binding sites); by degrading the 10 endogenous CREB; or by blocking the endogenous CREB from binding to a DNA or protein sequence required for wild-type CREB function. By way of example, one dominant negative CREB, referred to herein as KCREB, is described in Example 2. KCREB is a protein which binds to endogenous CREB and prevents its binding to CRE sequences. More specifically, KCREB is a naturally occurring CREB mutant in with an arginine to leucine substitution in the DNA binding domain (Walton et al., 1992, Mol. Endocrinol. 6:647-655). The mutation prevents KCREB from binding to DNA and as a result, KCREB:CREB heterodimers are transcriptionally inactive. The amino acid sequence of KCREB is represented herein as SEQ ID NO:8. SEQ ID NO:8 is encoded by a nucleic acid sequence represented herein as SEQ ID NO:7, however, as discussed above, it is to be understood that SEQ ID NO:8 can be encoded by a number of degenerate nucleic acid sequences. Other known dominant negative CREB proteins suitable for use in the present invention include, but are not limited to: A-CREB, CREB M1, ATF1RL, and a wild-type CREB DNA-binding fragment in the absence of a transactivation domain. A-CREB is a fusion protein comprising a designed acidic amphipathic extension onto the N-terminus of the CREB leucine zipper domain. The acidic extension of A-CREB interacts with the basic region of CREB forming a coiled-coil extension of the leucine zipper and thus prevents the basic region of endogenous, wild-type CREB from binding to DNA (Ahn et al., 1998, Mol. Cell. Biol. 18:967-977, incorporated herein by reference in its entirety). ATF1RL has a point mutation at the DNA binding domain of ATF and has been shown to block CREB-induced expression of a CRE reporter gene (Shimomura et al., 1998, J. Neurochem. 70:1029-1034, incorporated herein by reference in its entirety). CREB M1 is a nonphosphorylatable mutant of CREB, in which the serine at position 133 has been substituted with an alanine residue (described in detail in Somers et al., 1999, Mol. Endocrinol. 13:1364-1372, incorporated herein by reference in its entirety).

According to the present invention, a CREB protein having CREB biological activity, or a CREB protein having dominant negative CREB biological activity, is encoded by a nucleic acid sequence that is included in a recombinant nucleic acid molecule. It is noted that the present invention also encompasses the use of CREB protein mimetics and CREB synthetic mimetics, as discussed below in detail. A recombinant nucleic acid molecule of the present invention is a molecule that can include at least one of any nucleic acid sequence encoding a protein having CREB biological activity operatively linked to at least one of any transcription control sequence capable of effectively regulating expression of the nucleic acid molecule(s) in the cell to be transfected, examples of which are disclosed herein. Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. In addition, the phrase “recombinant molecule” primarily refers to a nucleic acid molecule operatively linked to a transcription control sequence, but can be used interchangeably with the phrase “nucleic acid molecule” which is administered to an animal.

In accordance with the present invention, an isolated nucleic acid molecule is a nucleic acid molecule that has been removed from its natural milieu (i.e., that has been subject to human manipulation). As such, “isolated” does not reflect the extent to which the nucleic acid molecule has been purified. An isolated nucleic acid molecule can include DNA, RNA, or derivatives of either DNA or RNA. There is no limit, other than a practical limit, on the maximal size of a nucleic acid molecule in that the nucleic acid molecule can include a portion of a gene, an entire gene, multiple genes, or portions thereof.

An isolated nucleic acid molecule of the present invention can be obtained from its natural source either as an entire (i.e., complete) gene or a portion thereof capable of forming a stable hybrid with that gene. Preferably, an isolated nucleic acid molecule is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on CREB biological activity. Allelic variants and protein homologues (e.g., proteins encoded by nucleic acid homologues) have been discussed in detail above.

A nucleic acid molecule homologue can be produced using a number of methods known to those skilled in the art (see, for example, Sambrook et al., ibid.). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene.

Preferred nucleic acid molecules according to the present invention are any isolated nucleic acid molecules which comprise a nucleic acid sequence encoding a CREB protein having CREB biological activity as described above. Preferred nucleic acid sequences are nucleic acid sequences which hybridize under stringent hybridization conditions with a nucleic acid sequence encoding a wild-type or constitutively active CREB protein as previously described herein, and which encode proteins having CREB biological activity. More preferred nucleic acid sequences are nucleic acid sequences encoding wild-type CREB proteins or constitutively active CREB proteins, including homologues thereof, as previously described herein. Even more preferred nucleic acid sequences are nucleic acid sequences encoding CREB proteins from human, rat and bovine. Additionally preferred nucleic acid sequences are nucleic acid sequences encoding a wild-type CREB, VP16-CREB, CREB DIEDML, or ATF-1 as previously described herein. Even more preferred nucleic acid sequences include nucleic acid sequences represented herein by SEQ ID NO:1, SEQ ID NO:3, SEQ ID NO:5, or SEQ ID NO:20, and fragments of such sequences which encode a protein having CREB biological activity.

In another embodiment, a preferred nucleic acid molecule according to the present invention is any isolated nucleic acid molecule which comprises a nucleic acid sequence encoding a CREB protein having CREB dominant negative biological activity as described above. Preferred nucleic acid sequences are nucleic acid sequences which hybridize under stringent hybridization conditions with a nucleic acid sequence encoding a dominant negative CREB protein as previously described herein, and which encode proteins having dominant negative CREB biological activity. More preferred nucleic acid sequences are nucleic acid sequences encoding dominant negative CREB proteins, including homologues thereof, as previously described herein. Additionally preferred nucleic acid sequences are nucleic acid sequences encoding a dominant negative CREB protein including KCREB, A-CREB, CREB M1, ATF1RL, and a wild-type CREB DNA-binding fragment in the absence of a transactivation domain as previously described herein. Even more preferred nucleic acid sequences encoding a protein having dominant negative CREB biological activity include nucleic acid sequences represented herein by SEQ ID NO:7, and fragments of such sequences which encode a protein having dominant negative CREB biological activity.

Knowing the nucleic acid sequences of certain nucleic acid molecules of the present invention allows one skilled in the art to, for example, (a) make copies of those nucleic acid molecules and/or (b) obtain nucleic acid molecules including at least a portion of such nucleic acid molecules (e.g., nucleic acid molecules including full-length genes, full-length coding regions, regulatory control sequences, truncated coding regions), Such nucleic acid molecules can be obtained in a variety of ways including traditional cloning techniques using oligonucleotide probes to screen appropriate libraries or DNA and PCR amplification of appropriate libraries or DNA using oligonucleotide primers. Preferred libraries to screen or from which to amplify nucleic acid molecule include mammalian genomic DNA libraries. Techniques to clone and amplify genes are disclosed, for example, in Sambrook et al., ibid.

A recombinant nucleic acid molecule includes a recombinant vector, which is any nucleic acid sequence, typically a heterologous sequence, which is operatively linked to the isolated nucleic acid molecule encoding a CREB protein, which is capable of enabling recombinant production of the CREB protein, and which is capable of delivering the nucleic acid molecule into a host cell according to the present invention. Such a vector can contain nucleic acid sequences that are not naturally found adjacent to the isolated nucleic acid molecules to be inserted into the vector. The vector can be either RNA or DNA, either prokaryotic or eukaryotic, and preferably in the present invention, is a virus or a plasmid. Recombinant vectors can be used in the cloning, sequencing, and/or otherwise manipulating of nucleic acid molecules. Recombinant vectors are preferably used in the expression of nucleic acid molecules, and can also be referred to as expression vectors. Preferred recombinant vectors are capable of being expressed in a transfected host cell, and particularly, in a transfected mammalian host cell in vivo.

In a recombinant molecule of the present invention, nucleic acid molecules are operatively linked to expression vectors containing regulatory sequences such as transcription control sequences, translation control sequences, origins of replication, and other regulatory sequences that are compatible with the host cell and that control the expression of nucleic acid molecules of the present invention. In particular, recombinant molecules of the present invention include nucleic acid molecules that are operatively linked to one or more transcription control sequences. The phrase “operatively linked” refers to linking a nucleic acid molecule to a transcription control sequence in a manner such that the molecule is able to be expressed when transfected (i.e., transformed, transduced or transfected) into a host cell.

Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in a host cell according to the present invention. A variety of suitable transcription control sequences are known to those skilled in the art. Preferred transcription control sequences include those which function in mammalian cells, with cell- or tissue-specific transcription control sequences being particularly preferred. Examples of preferred transcription control sequences include, but are not limited to, transcription control sequences useful for expression of a protein in adipocytes (e.g., aP2 and lipoprotein L (LPL) promoter, leptin promoter), transcription control sequences useful for expression of a protein in smooth muscle cells (e.g., α smooth muscle actin promoter and α smooth muscle myosin promoter), transcription control sequences useful for expression of a protein in cardiac myocytes (e.g., α-myosin heavy chain promoter, cardiac myosin light chain-2 promoter (MLC-2 promoter), β-myosin heavy chain promoter, cardiac troponin I promoter and cardiac troponin T promoter), and transcription control sequences useful for expression of a protein in neural cells (e.g., chromogranin A promoter, chromogranin B promoter, vgf promoter (promoter for gene encoding neurosecretory polypeptide, brain derived neurotrophic factor and neurotrophin 3), Thy-1 promoter, neuron restrictive enhancer elements). Particularly preferred transcription control sequences include inducible promoters, cell-specific promoters, tissue-specific promoters (e.g., insulin promoters) and enhancers. Suitable promoters for these and other cell types will be easily determined by those of skill in the art. Transcription control sequences of the present invention can also include naturally occurring transcription control sequences naturally associated with the protein to be expressed prior to isolation. In one embodiment, a transcription control sequence includes an inducible promoter.

Recombinant molecules of the present invention may also contain fusion sequences which lead to the expression of nucleic acid molecules as fusion proteins. Eukaryotic recombinant molecules may include intervening and/or untranslated sequences surrounding and/or within the nucleic acid sequences of nucleic acid molecules.

One type of recombinant vector useful in a recombinant nucleic acid molecule of the present invention is a recombinant viral vector. Such a vector includes a recombinant nucleic acid sequence encoding a CREB protein of the present invention that is packaged in a viral coat that can be expressed in a host cell in an animal or ex vivo after administration. A number of recombinant viral vectors can be used, including, but not limited to, those based on alphaviruses, poxviruses, adenoviruses, herpesviruses, lentiviruses, adeno-associated viruses and retroviruses. Particularly preferred viral vectors are those based on adenoviruses and adeno-associated viruses. Viral vectors suitable for gene delivery are well known in the art and can be selected by the skilled artisan for use in the present invention. A detailed discussion of current viral vectors is provided in “Molecular Biotechnology,” Second Edition, by Glick and Pasternak, ASM Press, Washington D.C., 1998, pp. 555-590, the entirety of which is incorporated herein by reference. A preferred viral vector for use in the present invention includes adenoviral vectors and adeno-associated viral vectors. In one embodiment, a preferred adenoviral vector is an adenovirus Ad5-derived vector which comprises SEQ ID NO:19.

For example, a retroviral vector, which is useful when it is desired to have a nucleic acid sequence inserted into the host genome for long term expression, can be packaged in the envelope protein of another virus so that it has the binding specificity and infection spectrum that are determined by the envelope protein (e.g., a pseudotyped virus). In addition, the envelope gene can be genetically engineered to include a DNA element that encodes and amino acid sequence that binds to a cell receptor to create a recombinant retrovirus that infects a specific cell type. Expression of the gene (i.e., the CREB gene) can be further controlled by the use of a cell or tissue-specific promoter. Retroviral vectors have been successfully used to transfect cells with a gene which is expressed and maintained in a variety of ex vivo systems

An adenoviral vector is a preferred vector for use in the present method. An adenoviral vector infects a wide range of nondividing human cells and has been used extensively in live vaccines without adverse side effects. Adenoviral vectors do not integrate into the host genome, and therefore, gene therapy using this system requires periodic administration, although methods have been described which extend the expression time of adenoviral transferred genes, such as administration of antibodies directed against T cell receptors at the site of expression (Sawchuk et al., 1996, Hum. Gene. Ther. 7:499-506). The efficiency of adenovirus-mediated gene delivery can be enhanced by developing a virus that preferentially infects a particular target cell. For example, a gene for the attachment fibers of adenovirus can be engineered to include a DNA element that encodes a protein domain that binds to a cell-specific receptor. Examples of successful in vivo delivery of genes has been demonstrated and is discussed in more detail below.

Yet another type of viral vector is based on adeno-associated viruses, which are small, nonpathogenic, single-stranded human viruses. This virus can integrate into a specific site on chromosome 19. This virus can carry a cloned insert of about 4.5 kb, and has typically been successfully used to express proteins in vivo from 70 days to at least 5 months. Demonstrating that the art is quickly advancing in the area of gene therapy, however, a recent publication by Bennett et al. reported efficient and stable transgene expression by adeno-associated viral vector transfer in vivo for greater than 1 year (Bennett et al., 1999, Proc. Natl. Acad. Sci. USA 96:9920-9925).

Another type of viral vector that is suitable for use in the present invention is a herpes simplex virus vector. Herpes simplex virus type 1 infects and persists within nondividing neuronal cells, and is therefore a suitable vector for targeting and transfecting cells of the central and peripheral nervous system with a CREB protein of the present invention. Preclinical trials in experimental animal models with such a vector has demonstrated that the vector can deliver genes to cells of both the brain and peripheral nervous system that are expressed and maintained for long periods of time.

One or more recombinant molecules of the present invention can be used to produce an encoded product (i.e., a protein having CREB biological activity or a protein having CREB dominant negative biological activity) useful in the method of the present invention. In one embodiment, an encoded product is produced by expressing a recombinant nucleic acid molecule as described herein under conditions effective to produce the protein. A preferred method to produce an encoded protein is by transfecting a host cell (i.e., a target cell) with one or more recombinant molecules to form a recombinant cell. Suitable host cells to transfect include any mammalian cell that can be transfected. Host cells can be either untransfected cells or cells that are already transfected with at least one nucleic acid molecule. Host cells according to the present invention can be any cell capable of producing a CREB protein as described herein. A preferred host cell includes any mammalian cell, and more preferably, mammalian adipocytes, vascular smooth muscle cells, cardiomyocytes (cardiac myocytes), hepatocytes, skeletal muscle cells, beta cells, pituitary cells, synovial lining cells, ovarian cells, testicular cells, fibroblasts, endothelial cells, and neural cells (including, but not limited to, hippocampal neural cells and cells of the cortex and/or basal ganglia).

According to the present invention, a host cell, also referred to as a target cell, in which a recombinant nucleic acid molecule encoding a CREB protein having CREB biological activity is to be expressed is selected from the group of: (a) a cell deficient in endogenous CREB expression; (b) a cell deficient in endogenous CREB biological activity; and (c) a cell having normal CREB expression and biological activity which is predisposed to become deficient in endogenous CREB expression or biological activity. As used herein, the term “target cell” refers to a cell to which a recombinant nucleic acid molecule of the present invention is selectively designed to be delivered. The term target cell does not necessarily restrict the delivery of a recombinant nucleic acid molecule only to the target cell and no other cell, but indicates that the delivery of the recombinant molecule, the expression of the recombinant molecule, or both, are specifically directed to a preselected host cell. Targeting delivery vehicles, including liposomes and viral vector systems are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., 1986, Biochemistry 25: 5500-6; Ho et al., 1987a, J Biol Chem 262: 13979-84; Ho et al., 1987b, J Biol Chem 262: 13973-8; and U.S. Pat. No. 4,957,735 to Huang et al., each of which is incorporated herein by reference in its entirety). Ways in which viral vectors can be modified to deliver a nucleic acid molecule to a target cell have been discussed above. Alternatively, the route of administration, as discussed below, can be used to target a specific cell or tissue. For example, intracoronary administration of an adenoviral vector has been shown to be effective for the delivery of a gene cardiac myocytes (Maurice et al., 1999, J. Clin. Invest. 104:21-29). Intravenous delivery of cholesterol-containing cationic liposomes has been shown to preferentially target pulmonary tissues (Liu et al., Nature Biotechnology 15:167, 1997), and effectively mediate transfer and expression of genes in vivo. Finally, a recombinant nucleic acid molecule can be selectively (i.e., preferentially, substantially exclusively) expressed in a target cell by selecting a transcription control sequence, and preferably, a promoter, which is selectively induced in the target cell and remains substantially inactive in non-target cells.

According to the present invention, a target cell which is deficient in endogenous CREB expression has a measurably reduced amount of CREB mRNA or CREB protein as compared to a normalized amount of CREB expression in the same cell type determined from a random population of normal patients. Similarly, a target cell which is deficient in endogenous CREB biological activity has a measurably reduced amount of CREB biological activity as compared to a normalized amount of CREB biological activity in the same cell type determined from a random population of normal patients. Reduced CREB biological activity can be a result of reduced CREB expression, but it is not limited to a reduction in CREB expression. Endogenous CREB expression is the level of CREB expression produced by the endogenous CREB gene (i.e., the CREB gene originating from within the host cell which has not been isolated). In most cell types, CREB is expressed at a level of at least about 10,000 molecules of CREB protein per cell, and more preferably, at least about 25,000 molecules of CREB protein per cell and even more preferably, at least about 50,000 molecules of CREB protein per cell. In a preferred embodiment, CREB is expressed at a level of from about 10,000 to 300,000 molecules of CREB protein per cell, and more preferably, from about 50,000 to about 200,000 molecules of CREB protein per cell. CREB biological activity has been described in detail above.

Methods of determining CREB expression and CREB biological activity in a given cell are known in the art and include, but are not limited to the methods described above. In patients with diseases including diabetes, macrovascular diseases, cardiomyopathy, osteoarthritis, post-angioplasty restenosis, amputations, spinal transsections, acute neuronal ischemia, and depression, a variety of cell types have been shown to have reduced CREB content (i.e., reduced CREB expression). Such cell types include, but are not limited to adipocytes in a patient that has diabetes; vascular smooth muscle cells in a patient that has diabetes, atherosclerosis, angina, acute myocardial infarction, stroke, pulmonary hypertension, amputation from peripheral vascular disease, or post-angioplasty restenosis; cardiomyocytes in a patient that has cardiomyopathy; synovial lining cells in a patient that has osteoarthritis; neural cells in a patient that has spinal cord transsection, acute neuronal ischemia, depression, Alzheimer's disease, and Parkinson's disease. A target cell that has normal CREB expression and/or biological activity, but which is predisposed to develop a deficiency in one and/or the other, is a target cell from a patient who has been clinically or genetically diagnosed as being susceptible to a disease or condition which can result in reduced CREB expression and/or biological activity, or who is showing early signs of such a disease or condition. Many of the diseases and conditions listed above are associated with genetic predictors and/or can be predicted based on the medical condition of the patient and any treatments to which the patient is currently exposed. For example, diabetes (the form primarily targeted by the present method is Type II, or adult onset, diabetes) is a disease for the development of which a patient can be predisposed. Predictors or factors influencing a patient's propensity to develop diabetes include genetic factors, family history, and obesity, age. Based on the present inventors' research, it is now known by disclosure herein that several cells of overtly diabetic patients have, or are expected to have, reduced or deficient CREB expression as a result of the diabetes and/or conditions related to the diabetes (e.g., atherosclerosis). Such cells include adipocytes, vascular smooth muscle cells, cardiomyocytes, hepatocytes, skeletal muscle cells, beta cells and pituitary cells. Therefore, a patient who is predisposed to develop diabetes is effectively predisposed to develop a deficiency in CREB expression and/or biological activity in certain target cell types. Such diseases and conditions are discussed in detail in the background and Examples section below.

In one embodiment of the present invention, the target cell for delivery of a nucleic acid sequence encoding a CREB protein having dominant negative CREB biological activity is an adipocyte in a patient who is to be treated to reduce total body adiposity, such as a patient who is obese. In such a patient, the endogenous CREB expression and biological activity is expected to be normal (in the absence of the patient having diabetes, for example), but in view of the present inventors' discovery, expression of a dominant negative CREB protein in the patient will cause lipid unloading and result in a decrease in total body adiposity.

In another embodiment of the present invention, the target cell for delivery of a nucleic acid sequence encoding a CREB protein having dominant negative CREB biological activity is a fibroblast and/or an endothelial cell in an area of neovascularization of a tumor. In the patient who has a tumor, the endogenous CREB expression and biological activity in an area of neovascularization is expected to be normal or high (in the absence of the patient having diabetes, for example), but in view of the present inventors' discovery, expression of a dominant negative CREB protein in the patient will be sufficient to inhibit proliferation and invasive potential of fibroblasts and/or endothelial cells into the tumor, resulting in inhibition of tumor neovascularization in the patient.

According to the method of the present invention, a host cell is preferably transfected in vivo (i.e., in a mammal) as a result of administration to a mammal of a recombinant nucleic acid molecule, or ex vivo, by removing cells from a mammal and transfecting the cells with a recombinant nucleic acid molecule ex vivo. Transfection of a nucleic acid molecule into a host cell according to the present invention can be accomplished by any method by which a nucleic acid molecule administered into the cell in vivo or ex vivo, and includes, but is not limited to, transfection, electroporation, microinjection, lipofection, adsorption, viral infection, naked DNA injection and protoplast fusion. Methods of administration are discussed in detail below.

It may be appreciated by one skilled in the art that use of recombinant DNA technologies can improve expression of transfected nucleic acid molecules by manipulating, for example, the duration of expression of the gene (i.e., recombinant nucleic acid molecule), the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into one or more host cell chromosomes, addition of vector stability sequences to plasmids, increasing the duration of expression of the recombinant molecule, substitutions or modifications of transcription control signals (e.g., promoters, operators, enhancers), substitutions or modifications of translational control signals (e.g., ribosome binding sites, Shine-Dalgarno sequences), modification of nucleic acid molecules of the present invention to correspond to the codon usage of the host cell, and deletion of sequences that destabilize transcripts. The activity of an expressed recombinant protein of the present invention may be improved by fragmenting, modifying, or derivatizing nucleic acid molecules encoding such a protein.

In one embodiment of the present invention, a recombinant nucleic acid molecule of the present invention is administered to a patient in a liposome delivery vehicle, whereby the nucleic acid sequence encoding the CREB protein enters the host cell (i.e., the target cell) by lipofection. A liposome delivery vehicle contains the recombinant nucleic acid molecule and delivers the molecules to a suitable site in a host recipient. According to the present invention, a liposome delivery vehicle comprises a lipid composition that is capable of delivering a recombinant nucleic acid molecule of the present invention, including both plasmids and viral vectors, to a suitable cell and/or tissue in a patient. A liposome delivery vehicle of the present invention comprises a lipid composition that is capable of fusing with the plasma membrane of the target cell to deliver the recombinant nucleic acid molecule into a cell.

A liposome delivery vehicle of the present invention can be modified to target a particular site in a mammal (i.e., a targeting liposome), thereby targeting and making use of a nucleic acid molecule of the present invention at that site. Suitable modifications include manipulating the chemical formula of the lipid portion of the delivery vehicle. Manipulating the chemical formula of the lipid portion of the delivery vehicle can elicit the extracellular or intracellular targeting of the delivery vehicle. For example, a chemical can be added to the lipid formula of a liposome that alters the charge of the lipid bilayer of the liposome so that the liposome fuses with particular cells having particular charge characteristics. Other targeting mechanisms include targeting a site by addition of exogenous targeting molecules (i.e., targeting agents) to a liposome (e.g., antibodies, soluble receptors or ligands).

A liposome delivery vehicle is preferably capable of remaining stable in a patient for a sufficient amount of time to deliver a nucleic acid molecule of the present invention to a preferred site in the patient (i.e., a target cell). A liposome delivery vehicle of the present invention is preferably stable in the patient into which it has been administered for at least about 30 minutes, more preferably for at least about 1 hour and even more preferably for at least about 24 hours. A preferred liposome delivery vehicle of the present invention is from about 0.01 microns to about 1 microns in size.

Suitable liposomes for use with the present invention include any liposome. Preferred liposomes of the present invention include those liposomes commonly used in, for example, gene delivery methods known to those of skill in the art. Preferred liposome delivery vehicles comprise multilamellar vesicle (MLV) lipids and extruded lipids. Methods for preparation of MLV's are well known in the art and are described, for example, in the Examples section. According to the present invention, “extruded lipids” are lipids which are prepared similarly to MLV lipids, but which are subsequently extruded through filters of decreasing size, as described in Templeton et al., 1997, Nature Biotech., 15:647-652, which is incorporated herein by reference in its entirety. Small unilamellar vesicle (SUV) lipids can also be used in the composition and method of the present invention. In one embodiment, liposome delivery vehicles comprise liposomes having a polycationic lipid composition (i.e., cationic liposomes) and/or liposomes having a cholesterol backbone conjugated to polyethylene glycol. In a preferred embodiment, liposome delivery vehicles useful in the present invention comprise one or more lipids selected from the group of DOTMA, DOTAP, DOTIM, DDAB, and cholesterol.

Preferably, the transfection efficiency of a nucleic acid:liposome complex of the present invention is at least about 1 picogram (pg) of protein expressed per milligram (mg) of total tissue protein per microgram (μg) of nucleic acid delivered. More preferably, the transfection efficiency of a nucleic acid:liposome complex of the present invention is at least about 10 pg of protein expressed per mg of total tissue protein per μg of nucleic acid delivered; and even more preferably, at least about 50 pg of protein expressed per mg of total tissue protein per μg of nucleic acid delivered; and most preferably, at least about 100 pg of protein expressed per mg of total tissue protein per μg of nucleic acid delivered.

Complexing a liposome with a nucleic acid molecule of the present invention can be achieved using methods standard in the art. A suitable concentration of a nucleic acid molecule of the present invention to add to a liposome includes a concentration effective for delivering a sufficient amount of recombinant nucleic acid molecule into a target cell of a patient such that the CREB protein encoded by the nucleic acid molecule can be expressed in a an amount effective to modulate the phenotype of the target cell. Preferably, from about 0.1 μg to about 10 μg of nucleic acid molecule of the present invention is combined with about 8 nmol liposomes. In one embodiment, the ratio of nucleic acids to lipids (μg nucleic acid:nmol lipids) in a composition of the present invention is preferably at least from about 1:10 to about 6:1 nucleic acid:lipid by weight (i.e., 1:10=1 μg nucleic acid: 10 nmol lipid).

According to the present invention, a recombinant nucleic acid molecule of the present invention is administered to a patient in a composition. In addition to the recombinant nucleic acid molecule, the composition can include, for example, a pharmaceutically acceptable carrier, which includes pharmaceutically acceptable excipients and/or delivery vehicles, for delivering the recombinant nucleic acid molecule to a patient (e.g., a liposome delivery vehicle). As used herein, a pharmaceutically acceptable carrier refers to any substance suitable for delivering a therapeutic composition useful in the method of the present invention to a suitable in vivo or ex vivo site. Preferred pharmaceutically acceptable carriers are capable of maintaining a recombinant nucleic acid molecule of the present invention in a form that, upon arrival of the nucleic acid molecule to a target cell, the nucleic acid molecule is capable of entering the cell and being expressed by the cell. Suitable excipients of the present invention include excipients or formularies that transport or help transport, but do not specifically target a nucleic acid molecule to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity.

Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal,—or o-cresol, formalin and benzol alcohol. Compositions of the present invention can be sterilized by conventional methods and/or lyophilized.

One type of pharmaceutically acceptable carrier includes a controlled release formulation that is capable of slowly releasing a composition of the present invention into an animal. As used herein, a controlled release formulation comprises recombinant nucleic acid molecule of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Suitable delivery vehicles have been previously described herein, and include, but are not limited to liposomes, viral vectors or other delivery vehicles, including ribozymes. Natural lipid-containing delivery vehicles include cells and cellular membranes. Artificial lipid-containing delivery vehicles include liposomes and micelles. As discussed above, a delivery vehicle of the present invention can be modified to target to a particular site in a patient, thereby targeting and making use of a nucleic acid molecule at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

It is noted that, although the composition can include any pharmaceutically acceptable carrier and virtually any other compound which may be suitable to administer to a patient for the purposes of providing a therapeutic benefit to the patient, it is a particularly beneficial aspect of the present invention, which was an unexpected discovery by the present inventors, that the composition of the present invention need not comprise any compound other than the recombinant nucleic acid molecule comprising a nucleic acid sequence encoding a CREB protein, (and a suitable delivery vehicle, if required, to delivery the recombinant nucleic acid molecule to a target cell). As discussed above, the present inventors have discovered that the transcription factor CREB is necessary and sufficient to induce a modulation of cell phenotype, and in many cells, to induce cell differentiation. Moreover, the present inventors have discovered that CREB expression and/or activity is decreased or absent in a variety of cell types associated with various diseases and conditions, and that restoration of CREB content and/or activity to these cells is sufficient to initiate or increase a differentiation program or phenotypic modulation program in the cells such that the phenotype of the cells is modulated. Therefore, the present inventors have discovered that several diseases and conditions can be prevented and/or ameliorated simply by regulating the content of CREB in a cell. According to the present invention, the phrase, “sufficient to modulate the phenotype,” with reference to the expression of recombinant CREB in a target cell, is to be interpreted to mean that regulation of CREB content in a target cell (i.e., by adding recombinant CREB having CREB biological activity or by inhibiting endogenous CREB by adding a recombinant CREB having dominant negative CREB biological activity), in the absence of an additional step of deliberately activating the recombinant CREB or the endogenous CREB in the cell, or manipulating the cell in any other way, is sufficient (i.e., enough, adequate) to modulate phenotype of the cell.

As discussed above, a composition of the present invention is administered to a patient in a manner effective to deliver the recombinant nucleic acid molecule comprising a nucleic acid sequence encoding a CREB protein having CREB biological activity to a target cell, whereby the target cell is transfected by the recombinant molecule and whereby the CREB protein is expressed in the target cell. Suitable administration protocols include any in vivo or ex vivo administration protocol.

According to the present invention, an effective administration protocol (i.e., administering a composition of the present invention in an effective manner) comprises suitable dose parameters and modes of administration that result in transfection and expression of a recombinant nucleic acid molecule encoding a CREB protein in a target cell of a patient, and subsequent modulation of the phenotype of the target cell, preferably so that the patient obtains some measurable, observable or perceived benefit from such administration. In some situations, where the target cell population is accessible for sampling, effective dose parameters can be determined using methods standard in the art for measuring whether the phenotype of a target cell has been modulated. Such methods include removing a sample of the target cell population from the patient prior to and after the recombinant nucleic acid molecule is administered, and measuring a change in the phenotype of the cell. Alternatively, effective dose parameters can be determined by experimentation using in vitro cell cultures, in vivo animal models, and eventually, clinical trials if the patient is human. Effective dose parameters can be determined using methods standard in the art for a particular disease or condition that the patient has or is at risk of developing. Such methods include, for example, determination of survival rates, side effects (i.e., toxicity) and progression or regression of disease.

According to the present invention, suitable methods of administering a composition comprising a recombinant nucleic acid molecule of the present invention to a patient include any route of in vivo administration that is suitable for delivering a recombinant nucleic acid molecule into a patient. The preferred routes of administration will be apparent to those of skill in the art, depending on the type of delivery vehicle used, the target cell population, and the disease or condition experienced by the patient. Preferred methods of in vivo administration include, but are not limited to, intravenous administration, intraperitoneal administration, intramuscular administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracerebral, nasal, oral, pulmonary administration, impregnation of a catheter, and direct injection into a tissue. In an embodiment where the target cells are in or near a tumor, a preferred route of administration is by direct injection into the tumor or tissue surrounding the tumor.

Ex vivo refers to performing part of the regulatory step outside of the patient, such as by transfecting a population of cells removed from a patient with a recombinant molecule comprising a nucleic acid sequence encoding a CREB protein according to the present invention under conditions such that the recombinant molecule is subsequently expressed by the transfected cell, and returning the transfected cells to the patient. Methods to achieve such transfection include, but are not limited to, transfection, viral infection, electroporation, lipofection, bacterial transfer, spheroplast fusion, and adsorption. Ex vivo methods are particularly suitable when the target cell can easily be removed from and returned to the patient.

Intravenous, intraperitoneal, and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering a recombinant nucleic acid molecule to a cell or tissue that is accessible by surgery, and particularly, on or near the surface of the body. Administration of a composition locally within the area of a target cell refers to injecting the composition centimeters and preferably, millimeters from the target cell or tissue.

Various methods of administration and delivery vehicles disclosed herein have been shown to be effective for delivery of a nucleic acid molecule to a target cell, whereby the nucleic acid molecule transfected the cell and was expressed. In many studies, successful delivery and expression of a heterologous gene was achieved in preferred cell types and/or using preferred delivery vehicles and routes of administration of the present invention. All of the publications discussed below and elsewhere herein with regard to gene delivery and delivery vehicles are incorporated herein by reference in their entirety. For example, using liposome delivery, U.S. Pat. No. 5,705,151, issued Jan. 6, 1998, to Dow et al. demonstrated the successful in vivo intravenous delivery of a nucleic acid molecule encoding a superantigen and a nucleic acid molecule encoding a cytokine in a cationic liposome delivery vehicle, whereby the encoded proteins were expressed in tissues of the animal, and particularly in pulmonary tissues. As discussed above, Liu et al., 1997, ibid. demonstrated that intravenous delivery of cholesterol-containing cationic liposomes containing genes preferentially targets pulmonary tissues and effectively mediates transfer and expression of the genes in vivo. Several publications by Dzau and collaborators demonstrate the successful in vivo delivery and expression of a gene into cells of the heart, including cardiac myocytes and fibroblasts and vascular smooth muscle cells using both naked DNA and Hemagglutinating virus of Japan-liposome delivery, administered by both incubation within the pericardium and infusion into a coronary artery (intracoronary delivery) (See, for example, Aoki et al., 1997, J. Mol. Cell, Cardiol. 29:949-959; Kaneda et al., 1997, Ann N.Y. Acad. Sci. 811 :299-308; and von der Leyen et al., 1995, Proc Natl Acad Sci USA 92:1137-1141).

As discussed above, delivery of numerous nucleic acid sequences has been accomplished by administration of viral vectors encoding the nucleic acid sequences. Using such vectors, successful delivery and expression has been achieved using ex vivo delivery (See, of many examples, retroviral vector; Blaese et al., 1995, Science 270:475-480; Bordignon et al., 1995, Science 270:470-475), nasal administration (CFTR-adenovirus-associated vector), intracoronary administration (adenoviral vector and Hemagglutinating virus of Japan, see above), intravenous administration (adeno-associated viral vector; Koeberl et al., 1997, Proc Natl Acad Sci USA 94:1426-1431). A publication by Maurice et al., 1999, ibid. demonstrated that an adenoviral vector encoding a β2-adrenergic receptor, administered by intracoronary delivery, resulted in diffuse multichamber myocardial expression of the gene in vivo, and subsequent significant increases in hemodynamic function and other improved physiological parameters. Levine et al. describe in vitro, ex vivo and in vivo delivery and expression of a gene to human adipocytes and rabbit adipocytes using an adenoviral vector and direct injection of the constructs into adipose tissue (Levine et al., 1998, J. Nutr. Sci. Vitaminol. 44:569-572).

In the area of neuronal gene delivery, multiple successful in vivo gene transfers have been reported. Millecamps et al. reported the targeting of adenoviral vectors to neurons using neuron restrictive enhancer elements placed upstream of the promoter for the transgene (phosphoglycerate promoter). Such vectors were administered to mice and rats intramuscularly and intracerebrally, respectively, resulting in successful neuronal-specific transfection and expression of the transgene in vivo (Millecamps et al., 1999, Nat. Biotechnol. 17:865-869). As discussed above, Bennett et al. reported the use of adeno-associated viral vector to deliver and express a gene by subretinal injection in the neural retina in vivo for greater than 1 year (Bennett, 1999, ibid.).

Gene delivery to synovial lining cells and articular joints has had similar successes. Oligino and colleagues report the use of a herpes simplex viral vector which is deficient for the immediate early genes, ICP4, 22 and 27, to deliver and express two different receptors in synovial lining cells in vivo (Oligino et al., 1999, Gene Ther. 6:1713-1720). The herpes vectors were administered by intraarticular injection. Kuboki et al. used adenoviral vector-mediated gene transfer and intraarticular injection to successfully and specifically express a gene in the temporomandibular joints of guinea pigs in vivo (Kuboki et al., 1999, Arch. Oral. Biol. 44:701-709). Apparailly and colleagues systemically administered adenoviral vectors encoding IL-10 to mice and demonstrated successful expression of the gene product and profound therapeutic effects in the treatment of experimentally induced arthritis (Apparailly et al., 1998, J. Immunol. 160:5213-5220). In another study, murine leukemia virus-based retroviral vector was used to deliver (by intraarticular injection) and express a human growth hormone gene both ex vivo and in vivo (Ghivizzani et al., 1997, Gene Ther. 4:977-982). This study showed that expression by in vivo gene transfer was at least equivalent to that of the ex vivo gene transfer. As discussed above, Sawchuk et al. has reported successful in vivo adenoviral vector delivery of a gene by intraarticular injection, and prolonged expression of the gene in the synovium by pretreatment of the joint with anti-T cell receptor monoclonal antibody (Sawchuk et al., 1996, ibid. Finally, it is noted that ex vivo gene transfer of human interleukin-1 receptor antagonist using a retrovirus has produced high level intraarticular expression and therapeutic efficacy in treatment of arthritis, and is now entering FDA approved human gene therapy trials (Evans and Robbins, 1996, Curr. Opin. Rheumatol. 8:230-234). Therefore, the state of the art in gene therapy has led the FDA to consider human gene therapy an appropriate strategy for the treatment of at least arthritis. Taken together, all of the above studies in gene therapy indicate that delivery and expression of a CREB encoding recombinant nucleic acid molecule according to the present invention is feasible.

Another method of delivery of recombinant molecules is in a non-targeting carrier (e.g., as “naked” DNA molecules, such as is taught, for example in Wolff et al., 1990, Science 247, 1465-1468). Such recombinant nucleic acid molecules are typically injected by direct or intramuscular administration. Recombinant nucleic acid molecules to be administered by naked DNA administration include a nucleic acid molecule of the present invention, and preferably includes a recombinant molecule of the present invention that preferably is replication, or otherwise amplification, competent. A naked nucleic acid reagent of the present invention can comprise one or more nucleic acid molecule of the present invention in the form of, for example, a dicistronic recombinant molecule. Naked nucleic acid delivery can include intramuscular, subcutaneous, intradermal, transdermal, intranasal and oral routes of administration, with direct injection into the target tissue being most preferred. A preferred single dose of a naked nucleic acid vaccine ranges from about 1 nanogram (ng) to about 100 μg, depending on the route of administration and/or method of delivery, as can be determined by those skilled in the art. Suitable delivery methods include, for example, by injection, as drops, aerosolized and/or topically. In one embodiment, pure DNA constructs cover the surface of gold particles (1 to 3 μm in diameter) and are propelled into skin cells or muscle with a “gene gun.”

As discussed above, the route of administration will depend on the delivery vehicle used and the target cell. For example, when the target cell is an adipocyte, preferred routes of administration include, but are not limited to intravenous administration, intraarterial administration, intraperitoneal administration and direct fat injection. When the target cell is a vascular smooth muscle cell, preferred routes of administration include, but are not limited to, intracoronary administration, intravenous administration, impregnation of an angioplasty catheter, intraarterial administration (e.g., carotid administration), and pulmonary administration. When the target cell is a cardiac myocyte, the preferred routes of administration include, but are not limited to, intracoronary administration, intravenous administration, intraventricular administration, and intraarterial administration. When the cells are synovial lining cells, preferred routes of administration include, but are not limited to, intravenous administration, intraarticular injection and intraarterial administration. When the cells are transplant cells, such as neuronal transplant cells for treatment of a patient with Parkinson's disease, the preferred route of administration is any method of ex vivo delivery. The above-described cell types and preferred routes of administration are provided as examples. Those of skill in the art will be able to determine preferred routes of administration for other cell types.

In accordance with the present invention, a suitable single dose of a recombinant nucleic acid molecule encoding a CREB protein as described herein is a dose that is capable of transfecting a host cell and being expressed in the host cell at a level sufficient, in the absence of the addition of any other factors or other manipulation of the host cell, to modulate the phenotype of the host cell when administered one or more times over a suitable time period. Doses can vary depending upon the cell type being targeted, the route of administration, the delivery vehicle used, and the disease or condition being treated.

In one embodiment, an appropriate single dose of a nucleic acid:liposome complex of the present invention is from about 0.1 μg to about 100 μg per kg body weight of the patient to which the complex is being administered. In another embodiment, an appropriate single dose is from about 1 μg to about 10 μg per kg body weight. In another embodiment, an appropriate single dose of nucleic acid:lipid complex is at least about 0.1 μg of nucleic acid, more preferably at least about 1 μg of nucleic acid, even more preferably at least about 10 μg of nucleic acid, even more preferably at least about 50 μg of nucleic acid, and even more preferably at least about 100 μg of nucleic acid.

Preferably, an appropriate single dose of a recombinant nucleic acid molecule encoding a CREB protein of the present invention results in at least about 1 pg of protein expressed per mg of total tissue protein per μg of nucleic acid delivered. More preferably, an appropriate single dose is a dose which results in at least about 10 pg of protein expressed per mg of total tissue protein per μg of nucleic acid delivered; and even more preferably, at least about 50 pg of protein expressed per mg of total tissue protein per μg of nucleic acid delivered; and most preferably, at least about 100 pg of protein expressed per mg of total tissue protein per μg of nucleic acid delivered.

In another embodiment, an appropriate single dose of a recombinant nucleic acid molecule encoding a CREB protein of the present invention results in an expression level in said cell of at least about 10,000 molecules of CREB per cell, and more preferably, at least about 25,000 molecules of CREB protein per cell and even more preferably, at least about 50,000 molecules of CREB protein per cell. In a preferred embodiment, CREB is expressed at a level of from about 10,000 to 300,000 molecules of CREB protein per cell, and more preferably, from about 50,000 to about 200,000 molecules of CREB protein per cell.

Compositions of the present invention can be administered to any animal patient, preferably to mammals, and more preferably to humans. According to the present invention, administration of a composition is useful to modulate the phenotype of a target cell. Typically, it is desirable to modulate the phenotype of a target cell to obtain a therapeutic benefit in the patient. Patients whom are suitable candidates for the method of the present invention using a recombinant nucleic acid molecule encoding a CREB protein having CREB biological activity include, but are not limited to, patients that have, or are at risk of developing (e.g., are predisposed to), diabetes (type II), atherosclerosis, angina, acute myocardial infarction, stroke, pulmonary hypertension, amputation from peripheral vascular disease, post-angioplasty restenosis, heart failure (including dilated cardiomyopathy or diabetic cardiomyopathy), osteoarthritis, spinal transsection, acute neuronal ischemia, Alzheimer's disease, Parkinson's disease, and/or depression.

According to the present invention, the method of the present invention is primarily directed to modulating the phenotype of a target cell in a patient with the added, but not required, goal of providing some therapeutic benefit to a patient. Modulating the phenotype of a target cell in a patient in the absence of obtaining some therapeutic benefit is useful for the purposes of determining factors involved (or not involved) in a disease and preparing a patient to more beneficially receive another therapeutic composition. In a preferred embodiment, however, the methods of the present invention are directed to the modulation of the phenotype of a target cell which is useful in providing some therapeutic benefit to a patient. As such, a therapeutic benefit is not necessarily a cure for a particular disease or condition, but rather, preferably encompasses a result which can include alleviation of the disease or condition, elimination of the disease or condition, reduction of a symptom associated with the disease or condition, prevention or alleviation of a secondary disease or condition resulting from the occurrence of a primary disease or condition (e.g., atherosclerosis resulting from diabetes), and/or prevention of the disease or condition. As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate disease symptoms, signs or causes. As such, to protect a patient from a disease includes both preventing disease occurrence (prophylactic treatment) and treating a patient that has a disease (therapeutic treatment). In particular, protecting a patient from a disease is accomplished by modulating the phenotype of a target cell in the patient by expressing a recombinant CREB protein having CREB biological activity (or a recombinant CREB protein having dominant negative CREB biological activity) such that a beneficial effect is obtained. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

By performing the method of the present invention, a recombinant CREB protein is expressed in a target cell, such expression being sufficient to modulate the phenotype of the target cell. In one embodiment of the present invention, the target cell is an adipocyte. In one embodiment, when the target cell is an adipocyte, and the adipocyte is deficient in CREB expression and/or biological activity, typically, the patient has or is at risk of developing diabetes (type II). The adipocyte in a patient that has diabetes, prior to the step of administering the recombinant nucleic acid molecule of the present invention, is generally deficient in CREB expression and/or biological activity as compared to an adipocyte from a patient that does not have and is not at risk of developing diabetes. In the patient at risk of developing diabetes, the CREB expression and biological activity may be normal (i.e., within the range of expression expected from the normal population of similar genetic background).

Expression of a recombinant CREB protein having CREB biological activity in the adipocyte can result in a change in expression of a protein in the adipocytes which can include, but is not limited to, an increase phosphoenolpyruvate carboxykinase (PEPCK), an increase Glut4, an increase in PPAR γ, an increase in fatty acid synthetase (FAS), an increase in fatty acid binding protein (FABP), an increase in C/EBP α, an increase in C/EBP β, and an increase in LPL and/or a decrease in PREF-1. Preferably, expression of a recombinant CREB protein having CREB biological activity in the adipocytes of the patient produces a result in the patient which includes, but is not limited to, decreased insulin resistance, normalized glucose control, and/or normalized lipid handling, as compared to any of these measurements prior to the conducting of the method of the present invention, or as compared to a patient who has not been administered the recombinant nucleic acid molecule encoding a CREB protein. Typically, prior to the step of administering or in the absence of the step of administering, a diabetic patient would experience increased insulin resistance, abnormal glucose control and abnormal lipid handling as compared to a range of parameters established from the non-diabetic population of similar genetic background. According to the present invention, determination of insulin resistance, glucose control and lipid handling can be readily accomplished by a clinician in the field of diabetes.

When the target cell is a vascular smooth muscle cell (VSMC), typically, the patient has or is at risk of developing diabetes (type II), atherosclerosis, angina, acute myocardial infarction, stroke, pulmonary hypertension, amputation from peripheral vascular disease, and/or post-angioplasty restenosis. Many of these conditions can be associated with the development of one or more of the other conditions. For example, a diabetic patient is at increased risk of developing atherosclerosis or suffering post-angioplasty restenosis. The VSMC in a patient, prior to the step of administering the recombinant nucleic acid molecule of the present invention, is generally deficient in CREB expression and/or biological activity as compared to a VSMC from a patient that does not have and is not at risk of developing one or more of the above conditions, or a similar condition that would be expected to affect VSMCs. In the patient at risk of developing such a condition, the CREB expression and biological activity may be normal (i.e., within the range of expression expected from the normal population of similar genetic background). In the embodiment wherein a patient is undergoing angioplasty, the step of administration is preferably performed concurrent with or substantially immediately following the angioplasty.

Expression of a recombinant CREB protein having CREB biological activity in the VSMC can result in a change in expression of a protein in the VSMC which can include, but is not limited to, a decrease in n vascular endothelial growth factor (VEGF), an increase in endothelial nitric oxide synthase (eNOS), an increase in tissue-plasminogen activator (tPA), a decrease in plasminogen activator inhibitor-1 (PAI-1), a decrease in heparin binding-endothelial growth factor (HB-EGF), and/or a decrease in inducible nitric oxide synthase (iNOS). Preferably, expression of a recombinant CREB protein having CREB biological activity in the VSMC of the patient produces a result which includes, but is not limited to, decreased proliferation of said cells, decreased migration of said cells, inhibition of cell cycle entry by said cells, increased contractility, decreased synthetic function and/or decreased cytokine expression, as compared to any of these measurements prior to the conducting of the method of the present invention. Typically, prior to the step of administering or in the absence of administering, a patient would experience increased proliferation of said cells, increased migration of said cells, cell cycle entry by said cells, decreased contractility, increased synthetic function and increased cytokine expression as compared to a range of parameters established from the normal population (i.e., the population not having the condition) of similar genetic background. According to the present invention, determination of these parameters can be readily accomplished by a clinician in the field. In addition, expression of a recombinant CREB protein having CREB biological activity in the VSMC of the patient produces a result which includes, but is not limited to, decreased susceptibility to post-angioplasty restenosis, reduced vessel occlusion, reduced atherosclerosis plaque formation, and decreased potential for pulmonary hypertension, as compared to a patient who has not been administered a recombinant nucleic acid molecule encoding a CREB protein, or as compared to prior to the step of administering.

When the target cell is a cardiomyocyte (also referred to as a cardiac myocyte), typically, the patient has or is at risk of developing heart failure, including, but not limited to dilated cardiomyopathy and diabetic cardiomyopathy. The cardiomyocytes in a patient, prior to the step of administering the recombinant nucleic acid molecule of the present invention, are generally deficient in CREB expression and/or biological activity as compared to a cardiomyocyte from a patient that does not have and is not at risk of developing heart failure. In the patient at risk of developing such a condition, the CREB expression and biological activity may be normal (i.e., within the range of expression expected from the normal population of similar genetic background).

Expression of a recombinant CREB protein having CREB biological activity in the cardiomyocytes can produce a result which can include, but is not limited to, expression of α-myosin heavy chain (α-MHC), spontaneous contraction, myocyte size and fibrillary structure, vacuolation and fibrosis, as compared to any of these measurements prior to the conducting of the method of the present invention. Typically, prior to the step of administering or in the absence of the step of administering, a patient would experience a decreased expression of α-myosin heavy chain (α-MHC), reduced spontaneous contraction and variations in myocyte size, vacuolation and microfibrillary structure, as compared to a range of parameters established from the normal population (i.e., the population not having the condition) of similar genetic background. Preferably, expression of a recombinant CREB protein having CREB biological activity in the cardiomyocytes results in decreased characteristics associated with dilated cardiomyopathy in the patient. According to the present invention, determination of these parameters can be readily accomplished by a clinician in the field.

When the target cell is a synovial lining cell, typically, the patient has or is at risk of developing osteoarthritis. The synovial lining cells in a patient, prior to the step of administering the recombinant nucleic acid molecule of the present invention, are generally deficient in CREB expression and/or biological activity as compared to a synovial lining cell from a patient that does not have and is not at risk of developing osteoarthritis. In the patient at risk of developing such a condition, the CREB expression and biological activity may be normal (i.e., within the range of expression expected from the normal population of similar genetic background).

Expression of a recombinant CREB protein having CREB biological activity in the synovial lining cells can produce a result which can include, but is not limited to, inhibition of synovitis, suppression of joint histopathology, inhibition of arthritis, as compared to any of these measurements prior to the conducting of the method of the present invention. Typically, prior to the step of administering or in the absence of the step of administering, a patient would experience increased synovitis, joint histopathology and arthritis as compared to a range of parameters established from the normal population (i.e., the population not having the condition) of similar genetic background. Preferably, expression of a recombinant CREB protein having CREB biological activity in the synovial lining cells results in decreased characteristics associated with osteoarthritis in the patient. According to the present invention, determination of these parameters can be readily accomplished by a clinician in the field.

When the target cell is a neural cell, typically, the patient has or is at risk of developing a disease or condition associated with a defect or damage to a neural cell which is associated with a deficiency in CREB expression and/or biological activity. Neural diseases or conditions that are associated with a CREB deficiency include, but are not limited to, spinal cord transsection, acute neuronal ischemia, Alzheimer's disease, Parkinson's disease and depression. In a patient that is has or is at risk of developing Alzheimer's disease, the neural target cell is a hippocampal neural cell. In a patient that has Parkinson's disease, the target cell is a neuronal cell transplant. When the patient has or is at risk of developing depression, the neural target cell are cells of the cortex and basal ganglia. The neural in a patient, prior to the step of administering the recombinant nucleic acid molecule of the present invention, are generally deficient in CREB expression and/or biological activity as compared to a neural cell from a patient that does not have and is not at risk of developing a neural condition or disease. In the patient at risk of developing such a condition, the CREB expression and biological activity may be normal (i.e., within the range of expression expected from the normal population of similar genetic background).

Expression of a recombinant CREB protein having CREB biological activity in the neural cells can produce a result which can include, but is not limited to, increased cell number, survival and/or expression of cell type-specific neurotransmitters, as compared to such parameters measured prior to the conducting of the method of the present invention. Typically, after the step of administering, a patient would experience improvement in memory (e.g., Alzheimer's), improved movement (Parkinson's), decreased depression, or decreased functional loss from acute ischemia (spinal cord injury/transsection or cardiovascular disease/condition), as compared to a range of parameters established from the normal population (i.e., the population not having the condition) of similar genetic background, and/or as compared to the experience of the patient prior to the step of administering or in the absence of the step of administering. Preferably, expression of a recombinant CREB protein having CREB biological activity in the neural cells results in decreased characteristics associated with the neural condition or disease in the patient. According to the present invention, determination of these parameters can be readily accomplished by a clinician in the field.

In one embodiment of the present invention, the target cell is an adipocyte in a patient that is in need of or desires to reduce total body adiposity. In one embodiment, the patient is obese. As used herein, the terms “obese” and “obesity” are used to refer to a condition in which an individual has a body mass index (BMI) of greater than 27 kilograms per square meter. It is to be understood, however, that this embodiment of the present method can be used to treat any individual who wishes to decrease total body adipocity, including for cosmetic reasons. In this embodiment, a recombinant nucleic acid molecule encoding a CREB protein having dominant negative CREB biological activity is administered to the patient. In the adipocyte in a patient that is to be treated to reduce total body adiposity, the CREB expression and biological activity may be normal (i.e., within the range of expression expected from the normal population of similar genetic background). Expression of a recombinant CREB protein having dominant negative CREB biological activity in the adipocyte preferably produces a result which can include: a reduction in the number of adipocytes in the patient, a reduction in fat stores in adipose tissue, a reduction in the discomfort and/or altered functions and detrimental conditions associated with such excessive fat stores, a reduction in the susceptibility or in the onset of the symptoms or complications of obesity, a reduction in body weight and/or a reduction in the rate of weight gain, as compared to any of these measurements prior to the conducting of the method of the present invention, or as compared to a patient who has not been administered the recombinant nucleic acid molecule encoding a CREB protein having dominant negative CREB biological activity. Reduction of adipocytes has been shown to be an effective method for treating obesity and excess body weight with minimal side effects (Flint, 1998, Biochem. Biophys. Res. Comm. 252:263-268). Flint showed that administration of an antibody to adipocytes resulted in a decrease in the number of adipocytes and favorable changes in body fat mass. Since the present inventors have shown that administration of a recombinant nucleic acid molecule encoding a CREB protein having dominant negative CREB biological activity leads to dedifferentiation of adipocytes with loss of triacylglycerol vesicles, even in the presence of insulin, such a loss in adipocytes and reduction in fat stores is expected to have an equal or better effect on body fat mass.

In another embodiment of the present invention, the target cell is a fibroblast or an endothelial cell in a neovessel of a tumor in a patient and the method is used to reduce tumor neovascularization. In this embodiment, a recombinant nucleic acid molecule encoding a CREB protein having dominant negative CREB biological activity is administered to the patient. In the fibroblasts or endothelial cells in or near a tumor in a patient that is to be treated to reduce tumor neovascularization, the CREB expression and biological activity may be normal or high (i.e., within the range of expression or higher than expected from the normal population of similar genetic background). It has been reported in the literature that CREB binding protein (CBP) plays an important role in vasculo-angiogenesis, and that in the absence of CBP, vasculo-angiogenesis is defective (Oike et al., Am. Soc. Hematol. 9:2771-2779, 1999). The present inventors have found that in atherosclerosis in humans, vessels have neovascularization in the adventitia (outer wall of the artery) in which fibroblasts and endothelial cells have a high CREB content. Therefore, the CREB content in these cells in the neovasculature are correlated with the proliferative and invasive potential of such cells. Given these data, the present inventors believe that expression of a recombinant CREB protein having dominant negative CREB biological activity in tumor cells of a patient will inhibit the proliferative and invasive potential of fibroblasts and endothelial cells in or near the neovessels of the tumor, thereby inhibiting neovascularization of the tumor. Therefore, administration of the dominant negative CREB protein is believed to be an effective anti-tumor treatment.

Another embodiment of the present invention relates to a method for restoring the ability of a cell to differentiate. This method includes the step of transfecting a cell that is deficient in CREB expression or CREB biological activity with a recombinant nucleic acid molecule comprising a nucleic acid sequence encoding a cyclic-AMP responsive element binding (CREB) protein having CREB biological activity, such that the CREB protein encoded by the recombinant nucleic acid molecule is expressed in the cell. Prior to the step of transfecting, the cell is not fully differentiated. The various components and protocols by which this method can be achieved have been previously described herein.

Yet another embodiment of the present invention relates to a method to treat diabetes in a patient. Such a method includes the step of administering to a patient a composition comprising a recombinant nucleic acid molecule comprising a nucleic acid sequence encoding a cyclic-AMP responsive element binding (CREB) protein having CREB biological activity operatively linked to a transcription control sequence. The CREB protein is expressed by the recombinant nucleic acid molecule in target cells in the patient selected from adipocytes and vascular smooth muscle cells. Expression of the CREB protein in the target cells is sufficient to modulate the phenotype of said cells. Preferably, expression of the CREB protein in the target cells produces a result in the patient selected from the group of increased glucose control, decreased insulin resistance, reduced post-angioplasty restenosis, reduced atherosclerosis, reduced total body adiposity, normalization of lipid handling and/or normalization of hepatic glucose and protein handling. The various components and protocols by which this method can be achieved have been previously described herein.

Another embodiment of the present invention relates to a method to modulate the phenotype of adipocytes in a patient who has or is at risk for developing diabetes, such method including the step of administering to the patient a composition comprising a recombinant nucleic acid molecule comprising a nucleic acid sequence encoding a cyclic-AMP responsive element binding (CREB) protein having CREB biological activity operatively linked to a transcription control sequence. The CREB protein is expressed by the recombinant nucleic acid molecule in adipocytes of the patient, and the expression of the CREB protein in the adipocytes is sufficient to modulate the phenotype of the adipocytes. The various components and protocols by which this method can be achieved have been previously described herein.

Yet another embodiment of the present invention relates to a method to modulate the phenotype of vascular smooth muscle cells in a patient, comprising administering to the patient a composition comprising a recombinant nucleic acid molecule comprising a nucleic acid sequence encoding a cyclic-AMP responsive element binding (CREB) protein having CREB biological activity operatively linked to a transcription control sequence. The CREB protein is expressed by the recombinant nucleic acid molecule in vascular smooth muscle cells of the patient; and the expression of the CREB protein in the vascular smooth muscle cells is sufficient to modulate the phenotype of the vascular smooth muscle cells. In one embodiment, the patient has or is at risk for developing a condition selected from the group of diabetes, atherosclerosis, angina, acute myocardial infarction, stroke, amputation from peripheral vascular disease, post-angioplasty restenosis, and pulmonary hypertension. The various components and protocols by which this method can be achieved have been previously described herein.

Yet another embodiment of the present invention is a method to modulate cardiomyocyte phenotype in a patient, comprising administering to a patient a composition comprising a recombinant nucleic acid molecule comprising a nucleic acid sequence encoding a cyclic-AMP responsive element binding (CREB) protein having CREB biological activity operatively linked to a transcription control sequence. The CREB protein is expressed by the recombinant nucleic acid molecule in cardiomyocytes of the patient, and the expression of the CREB protein in the cardiomyocytes is sufficient to modulate the phenotype of the cardiomyocytes. Preferably, the patient has or is at risk for developing dilated cardiomyopathy or diabetic cardiomyopathy. The various components and protocols by which this method can be achieved have been previously described herein.

One embodiment of the present invention is a method to modulate neural cell phenotype. This method includes the step of administering to a patient a composition comprising a recombinant nucleic acid molecule comprising a nucleic acid sequence encoding a cyclic-AMP responsive element binding (CREB) protein having CREB biological activity operatively linked to a transcription control sequence. The CREB protein is expressed by the recombinant nucleic acid molecule in neural cells of the patient, and the expression of the CREB protein in the neural cells is sufficient to modulate the phenotype of the neural cells. In one embodiment, the patient has or is at risk of developing Alzheimer's disease, and the neural cells are hippocampal neurons. In another embodiment, the patient has or is at risk of developing Parkinson's disease, and the neural cells are dopaminergic neural transplant cells, and the step of administering is performed ex vivo, prior to transplantation of the dopaminergic neural transplant cells into said patient. In other embodiments, the patient has a spinal cord transsection, has or is at risk for developing acute neuronal ischemia, or has or is at risk for developing depression. In this latter embodiment, the neural cells are selected from the group of cortex cells and basal ganglia cells. The various components and protocols by which this method can be achieved have been previously described herein.

Yet another embodiment of the present invention is a method to treat osteoarthritis. such method includes the step of administering to a patient that has or is at risk of developing osteoarthritis a composition comprising a recombinant nucleic acid molecule comprising a nucleic acid sequence encoding a cyclic-AMP responsive element binding (CREB) protein having CREB biological activity operatively linked to a transcription control sequence. The CREB protein is expressed by the recombinant nucleic acid molecule in synovial lining cells of the patient, and the expression of the CREB protein in the synovial lining cells is sufficient to modulate the phenotype of the synovial lining cells. The various components and protocols by which this method can be achieved have been previously described herein.

Yet another embodiment of the present invention is a method to inhibit tumor neovascularization in a patient. This method includes the step of administering to the patient a composition comprising a recombinant nucleic acid molecule comprising a nucleic acid sequence encoding a cyclic-AMP responsive element binding (CREB) protein having dominant negative CREB biological activity operatively linked to a transcription control sequence. The CREB protein is expressed by said recombinant nucleic acid molecule in fibroblasts and/or endothelial cells in or near a tumor in the patient, and the expression of the CREB protein in the cells is sufficient to modulate the phenotype of the cells, resulting in an inhibition of tumor neovascularization in said patient. Preferably, the proliferative ability and potential for tumor invasiveness of the cells is reduced by expression of the dominant negative CREB protein. In this embodiment, the CREB protein having dominant negative CREB biological activity is selected from the group of KCREB, A-CREB, CREB M1, ATF1RL, and a wild-type CREB DNA-binding fragment. Preferred dominant negative CREB proteins are described in detail above. The various components and protocols by which this method can be achieved have been previously described herein.

Yet another embodiment of the present invention is a method to decrease total body adiposity, comprising administering to a patient a composition comprising a recombinant nucleic acid molecule comprising a nucleic acid sequence encoding a cyclic-AMP responsive element binding (CREB) protein having dominant negative CREB biological activity operatively linked to a transcription control sequence. The CREB protein is expressed by said recombinant nucleic acid molecule in adipocytes of said patient, and the expression of the CREB protein in the adipocytes is sufficient to inhibit differentiation of the adipocytes, resulting in a decrease in total body adiposity in the patient. The various components and protocols by which this method can be achieved have been previously described herein.

One embodiment of the present invention relates to a method to modulate the phenotype of a target cell population in a patient, comprising administering to a patient a composition comprising a synthetic or peptide mimetic of a cyclic-AMP responsive element binding (CREB) protein, also referred to herein as a CREB mimetic. The mimetic is introduced into target cells in the patient. The target cells are selected from the group of: (a) cells deficient in endogenous CREB expression; (b) cells deficient in endogenous CREB biological activity: and, (c) cells having normal endogenous CREB expression and biological activity which are predisposed to become deficient in endogenous CREB expression or biological activity. The introduction of the CREB mimetic into the target cell is sufficient to modulate the phenotype of the cells.

As used herein, the terms “mimetic” and “mimetope” can be used interchangeably. According to the present invention, a mimetic of CREB protein refers to any compound that is able to mimic the biological action of CREB protein, often because the mimetic has a three dimensional structure that mimics the three dimensional structure of the CREB protein. As such, a CREB protein homologue as described above can be one type of mimetic, however, mimetics encompassed by the present invention can also include molecules that are much less similar to the native protein at an amino acid level, as well as non-protein molecules (i.e., synthetic mimetics). Mimetics can include, but are not limited to: peptides that have substantial modifications which, for example, decrease their susceptibility to degradation; anti-idiotypic and/or catalytic antibodies, or fragments thereof; non-proteinaceous portions of an isolated protein (e.g., carbohydrate structures); or synthetic or natural organic molecules, including nucleic acids.

Mimetics can be designed using computer-generated three dimensional structures of CREB proteins, for example. Mimetics can also be obtained by generating random samples of molecules, such as oligonucleotides, peptides or other organic or inorganic molecules, and screening such samples for CREB protein biological activity or affinity for a binding partner of a naturally occurring CREB protein (e.g., a DNA binding site, CRE, or a protein binding site).

Also included in the present invention are CREB mimetics having increased or decreased stability and/or increased or decreased biological activity compared to an unmodified CREB protein. As used herein, the term “unmodified CREB protein” refers to a CREB protein as described herein that has not been intentionally subjected to either random or site-directed (i.e., targeted) mutagenesis or which has not undergone naturally occurring mutagenesis. The present invention includes an isolated peptide mimetic or synthetic mimetic having measurable CREB protein biological activity and modified stability compared to an unmodified CREB protein. The present invention also includes a peptide mimetic or synthetic mimetic having modified CREB protein biological activity compared to a naturally occurring, unmodified CREB protein.

According to the present invention, the peptide and synthetic mimetics of CREB proteins can be designed by creating a new chemical or biological (e.g. protein, peptide, antibody, antisense, ribozyme) compound or searching databases of libraries of known compounds (e.g., a compound listed in a computational screening database containing three dimensional structures of known compounds). Designing can also be performed by simulating chemical or biological compounds having substitute moieties at certain structural features. The step of designing can include selecting a compound based on a known function of the compound. A preferred step of designing comprises computational screening of one or more databases of compounds in which the three dimensional structure of the compound is known and is interacted (e.g., docked, aligned, matched, interfaced) with the three dimensional structure (or predicted three dimensional structure) of CREB protein by computer (e.g. as described by Humblet and Dunbar, Animal Reports in Medicinal Chemistry, vol. 28, pp. 275-283, 1993, M Venuti, ed., Academic Press). Methods to synthesize suitable chemical or biological compounds are known to those of skill in the art and depend upon the structure of the chemical or other molecule being synthesized. Methods to evaluate the bioactivity of the synthesized compound depend upon the bioactivity of the compound (e.g., inhibitory or stimulatory) and are disclosed herein.

Various methods of drug design are disclosed in Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In a method of drug design, it is not always necessary to align a candidate compound (i.e., a compound being analyzed in, for example, a computational screening method) to each residue in a target site. Suitable candidate compounds can align to a subset of residues described for a target site. Preferably, a candidate compound comprises a conformation that promotes the formation of covalent or noncovalent crosslinking between the target site and the candidate compound. Preferably, a candidate compound binds to a surface adjacent to a target site to provide an additional site of interaction in a complex. When designing an antagonist (e.g., a dominant negative CREB mimetic), the antagonist should bind with sufficient affinity to the binding site or to substantially prohibit a ligand (i.e., a molecule that specifically binds to the target site) from binding to a target area. It will be appreciated by one of skill in the art that it is not necessary that the complementarity between a candidate compound and a target site extend over all residues specified here in order to inhibit or promote binding of a ligand.

In general, the design of a chemical or biological compound possessing stereochemical complementarity can be accomplished by means of techniques that optimize, chemically or geometrically, the “fit” between a compound and a target site. Such techniques are disclosed by, for example, Sheridan and Venkataraghavan, Acc. Chem Res., vol. 20, p. 322, 1987: Goodford, J. Med. Chem., vol. 27, p. 557, 1984; Beddell, Chem. Soc. Reviews, vol. 279, 1985; Hol, Angew. Chem., vol. 25, p. 767, 1986; and Verlinde and Hol, Structure, vol. 2, p. 577, 1994, each of which are incorporated by this reference herein in their entirety.

The following examples are provided for the purposes of illustration and are not intended to limit the scope of the present invention.

### EXAMPLES

### Materials

Materials for the following experiments were obtained as follows.

An expression vector (pRSV-KCREB) for the dominant negative CREB inhibitor protein, KCREB, was provided by Dr. Richard Goodman (Oregon Health Sciences Univ., Portland, Oreg.). CREB and P-CREB specific antibodies were purchased from New England Biolabs (Beverly, Mass.). Antibodies to PPARγ2, RXRα, CEBPs α and γ, and VP16 were purchased from Santa Cruz Biotechnology (Santa Cruz, Calif.). Cell Titer 96 AQ reagents were from Promega (Madison, Wis.), and the Ecdysone Inducible Expression System and total RNA isolation reagents were from Invitrogen (Carlsbad, Calif.).

A biotinylated, 60 base oligonucleotide complimentary to the mouse Fatty Acid Binding Protein (FABP or aP2/422, bases 1-60 of the open reading frame (SEQ ID NO:18)), and 20 base pair double stranded oligonucleotides for gel retardation assays were purchased from Gene Link (Thornwood, N.Y.). Other plasmids, luciferase assay reagents, and all other chemicals and materials have been described in (Klemm et al., 1998, J. Biol. Chem., 273:917-923), which is incorporated herein by reference in its entirety.

### Example 1

The following example demonstrates that CREB is constitutively expressed prior to and during adipogenesis and is regulated by differentiation inducing agents.

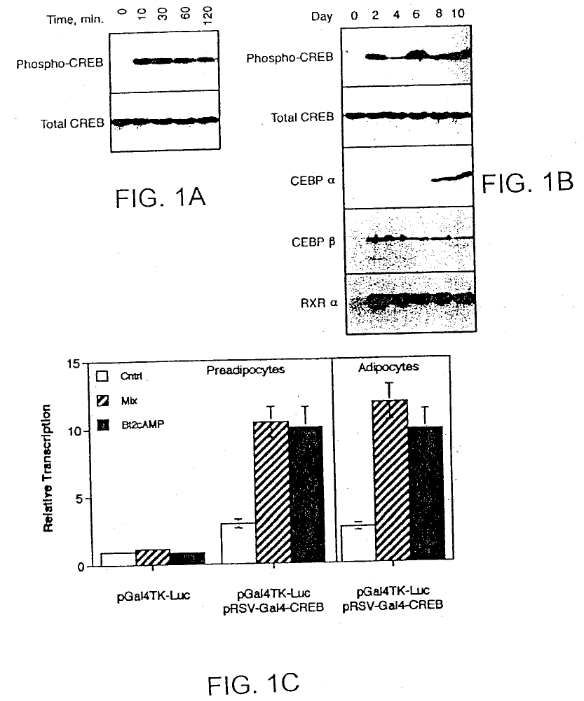

Maximal differentiation of 3T3-L1 preadipocytes to mature adipocytes requires the addition of a mixture of insulin, a glucocorticoid, and a CAMP mimetic (MacDougald et al., 1995, Annu. Rev. Biochem., 64:345-373). However, Green and Kehinde (Green et al., 1975, Cell, 5:19-27) demonstrated that 3T3-L1 cells would undergo adipogenesis and accumulate triacylglycerol when treated with insulin or cAMP mimetics alone. The present inventors had previously observed increases in CREB phosphorylation and transcriptional activity in 3T3-L1 cells treated with these agents, leading them to investigate whether CREB may play a role in adipogenesis. The first clue that CREB might be involved in adipocyte differentiation was observed in assays in which total CREB (unphosphorylated plus phosphorylated) protein and Ser133 phosphorylated CREB (phospho-CREB or P-CREB) were measured in NIH 3T3-L1 cells (FIG. 1). In the experiment shown in FIG. 1A, NIH 3T3-L1 preadipocytes, grown to confluency, were refed with complete growth medium containing 1 μg/ml insulin, 1 uM dexamethasone, and 0.5 mM Bt2cAMP for the times indicated above each lane. Approximately 25 ug of cell lysate protein from each sample were separated on 10% acrylamide-SDS gels and transferred to nitrocellulose membranes. Duplicate membranes were subjected to Western analysis using antibodies specific for P-CREB or total CREB (CREB) as indicated.

In the experiment shown in FIG. 1B, preadipocytes were grown to confluency and then refed with medium containing insulin, dexamethasone, and Bt2cAMP for 48 hours. The cells were then refed every two days with medium containing 1 ug/ml insulin. Cell lysates were prepared on the days indicated above each lane and 25 ug of lysate protein from each sample was separated on 10% polyacrylamide-SDS gels and transferred to nitrocellulose membranes. Individual membranes were probed with antibodies specific for P-CREB, total CREB (CREB), CEBPs α and β, and RXR α, as indicated. Briefly, the nitrocellulose blots were blocked with phosphate buffered saline-containing 5% dry milk and 0.1% Tween 20, and then treated with antibodies that recognize phosphorylated CREB (P-CREB), total CREB, CEBPs α and β, RXR α, PPARγ2, or VP16. The blots were washed and subsequently treated with goat anti-rabbit IgG conjugated to alkaline phosphatase (for CREB, P-CREB, CEBP α and γ, RXR α and PPARγ2 antibodies)or anti-goat/alkaline phosphatase conjugate (VP16 antibody). After the blots were washed, specific immune complexes were visualized with bromo-chloro-indoyl-phosphate and nitro blue tetrazolium.

CREB was present in 3T3-L1 fibroblasts prior to the induction of adipogenesis, and throughout the differentiation process at relatively stable levels (FIG. 1A and 1B, total CREB panels). This is in sharp contrast to other adipocyte-specific transcription factors like CEBPs α and β, and RXR α which are undetectable in untreated preadipocytes (FIG. 1B, Day 0). CEBP β and RXR α first become detectable in our experiments on day 2 of differentiation, while CEBP α does not appear until day 8.

Not only was CREB present in 3T3-L1 cells before and during adipogenesis, but phosphorylation of CREB was rapidly stimulated in cells treated with a differentiation-inducing mixture containing insulin, Bt₂cAMP, and dexamethasone (FIG. 1A). Phospho-CREB (P-CREB) levels increased approximately 20-fold within 10 minutes of treatment, remained elevated for another 20 minutes, and then began to decline slowly. Variations in CREB phosphorylation were also noted during the 10 day differentiation process (FIG. 1B) and appear to reflect changes in CREB phosphorylation due to refeeding of the cells with insulin-supplemented, serum-containing medium.

Next, the ability the ability of differentiation-inducing agents to regulate CREB transcriptional activity was assessed. For these experiments, 3T3-L1 preadipocytes or mature adipocytes were transfected with a plasmid from which a chimeric protein composed of the CREB transactivation domain (amino acids 1-261 of CREB-327) linked to the Gal4 DNA binding domain (amino acids 1-174) was expressed. Transcriptional activity of this chimeric protein was measured by co-transfecting the cells with a plasmid containing a Gal4-responsive promoter linked to a luciferase reporter gene (pGal4TK-Luc). Twenty four hours after transfection, the cells were treated with 0.5 mM Bt₂cAMP alone, or a mixture of 1 ug/ml insulin, 1 uM dexamethasone, and 0.5 mM Bt₂cAMP for 4 hours. Control cells received no treatment. Luciferase levels were measured in cell lysates as an index of transcription from the Gal4-TK promoter. Luciferase and β-galactosidase assays were performed as previously described (Klemm et al., 1998, J. Biol. Chem., 273:917-923). Levels of transcription are shown relative to levels measured in untreated control cells transfected with pGal4TK-Luc alone. As shown in FIG. 1C, transcription from the Gal4-responsive promoter was unaffected by any treatment in the absence of Gal4-CREB protein. However, in either preadipocytes or mature adipocytes expressing Gal4-CREB, the differentiation-inducing mixture of insulin, Bt₂cAMP, and dexamethasone or, Bt₂cAMP alone stimulated transcription from the Gal4-responsive promoter by 10 to 12-fold. The ability of adipogenesis-inducing agents to stimulate both CREB phosphorylation and transcriptional activity, and the constitutive expression of CREB prior to and during differentiation supported the hypothesis that CREB might play a role in initiating and maintaining the adipocyte differentiation program.

### Example 2

The following example demonstrates the generation of stably transfected 3T3-L1 fibroblasts that inducibly express constitutively active and dominant negative forms of CREB.

To directly assess the participation of CREB in adipogenesis, stably transfected 3T3-L1 cell lines were generated in which the expression of constitutively active or dominant negative forms of CREB could be induced with the insect hormone homologue, muristerone. This system allowed the direct modulation of CREB transcriptional activity without relying on pharmacological agents that might regulate other signaling pathways and transcription factors. Constitutively active CREB consisted of the transactivation domain of the viral VP16 protein (amino acids 412-490) linked to the CREB DNA binding domain (amino acids 217-327), the resulting protein of which is represented herein by SEQ ID NO:6. KCREB, a protein which binds to endogenous CREB and prevents its binding to CRE sequences (Walton et al., 1992, Mol. Endo., 6:647-655) was employed as the dominant negative CREB. Two clones expressing VP16-CREB (2-4 and 9-7) and two clones expressing KCREB (2-1 and 2-10) in response to muristerone were isolated and characterized.

Briefly, the Edison-Inducible Expression System was employed to prepare stably transfected 3T3-L1 cells in which the expression of VP16-CREB and KCREB could be induced. The open reading frame for KCREB was isolated from the plasmid, PRSV-KCREB as HindIII-EcoRI fragment. This fragment was subjected to PCR with a 5′ primer that introduced a consensus Kozak translation initiation sequence immediately upstream of the first methionine codon. The resulting PCR product was ligated into the Hind III and EcoRI sites of the plasmid, pIND. The open reading frame for VP16 (amino acids 412-490) was excised from the plasmid, pVP16 (Arthur Gutierrez-Hartman, University of Colorado Health Sciences Center, Denver, Colo.) as a HindIII-BamHI fragment. This fragment was subjected to PCR to introduce a Kozak sequence immediately upstream of the translation start site. This fragment was directly ligated to a BglII-EcoRI fragment containing the DNA binding domain (amino acids 217-327) of CREB-327 excised from the plasmid pRSET-CREB (James Hoeffier, Invitrogen, Carlsbad, Calif.). This chimeric VP16-CREB gene was ligated into the HindIII and EcoRI sites of pIND. The resulting plasmids were confirmed by restriction enzyme mapping and sequencing.

3T3-L1 fibroblasts were passaged and treated with differentiation-inducing agents as previously described (Klemm et al., 1998, J. Biol. Chem., 273:917-923), incorporated herein by reference in its entirety. Plates of 3T3-L1 fibroblasts and adipocytes were grown to 70-80% confluency and transfected with the above-described plasmids with Superfect Reagent (Oiagen, Valencia, Calif.) according to the manufacturer's recommendations. Cells stably transfected with the plasmid, pVgRXR were selected for in conventional medium containing 500 ug/mi Zeocin, and cells stably transfected with pIND-VP16-CREB, pIND-KCREB or pIND-LacZ plasmids were selected for in medium containing 500 ug/ml Geneticin. Large, rapidly growing, well separated colonies were isolated 10 to 12 days after selection was begun with either antibiotic. Isolated clones were passaged in low glucose DMEM containing 10% FCS, 1 mM L-glutamine, and 500 ug/ml each of Zeocin and Geneticin. VP16-CREB or KCREB expression was induced through the addition of 10 uM Muristerone to the growth. The effect of VP16-CREB and KCREB expression on 3T3-L1 proliferation was assessed by measuring cell number with the Cell-Titer 96 Aq reagent system (Promega Corp., Madison, Wis.).

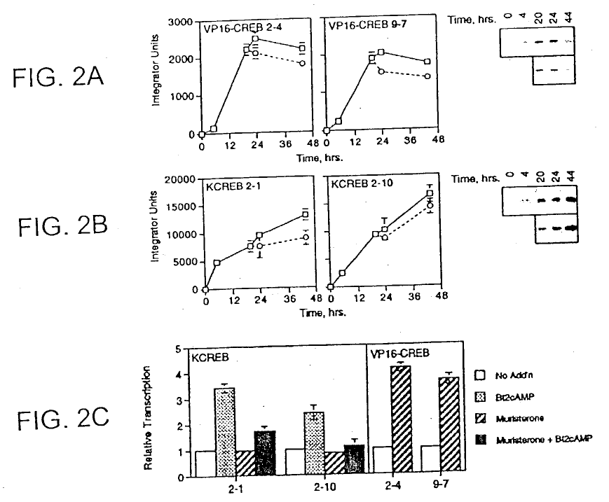

The muristerone-induced appearance of VP16-CREB protein in clones 2-4 and 9-7 was examined by Western blot analysis using antibodies specific for VP16 (FIG. 2A), and the kinetics of KCREB induction were followed by Western blots analysis using antibodies to total CREB (level corrected for endogenous CREB content in untreated cells) (FIG. 2B). In these experiments, the expression of VP16-CREB or KCREB was monitored in these clonal cell lines versus time following treatment with muristerone at a final concentration of 10 uM. At 20 hours, duplicate wells of cells were refed with medium lacking muristerone (levels indicated by dashed lines) for comparison to cells in medium with muristerone (solid lines). Levels of VP16-CREB and KCREB were measured by separating 25 ug of protein from lysate prepared at the times shown on 10% acrylamide-SDS gels. Proteins were transferred from the gels to nitrocellulose membranes subsequently probed with antibodies to VP16 (for VP16-CREB) or CREB (for KCREB). Since the CREB antibody detected both KCREB and endogenous CREB proteins, levels of KCREB expression were corrected for endogenous CREB levels measured in untreated cells (not shown). The optical densities of the bands on the blots was determined using Scan Analysis Software after the blots had been scanned into a Macintosh computer via an Agfa DuoScan T2100 scanner. A representative blot for each protein is displayed to the right of the graphs.

FIG. 2A shows that VP16-CREB levels increased slowly over the first four hours following muristerone addition in both cell lines, and reached maximal levels in 20 to 24 hours. Thereafter, protein levels decreased slowly even in the presence of muristerone. Removal of muristerone from the cells slightly increased the rate of VP16-CREB disappearance. The kinetics of KCREB induction differed significantly from VP16-CREB expression (FIG. 2B). KCREB levels increased much more rapidly than VP16-CREB in both clones, and continued to rise throughout the course of the assay. Although removal of muristerone at 20 hours decreased the rate of KCREB expression, KCREB levels continued to increase.

To test the ability of KCREB and VP16-CREB expression to influence gene transcription, a plasmid containing a truncated, CRE-containing portion of the phosphoenolpyruvate carboxykinase (PEPCK) gene promoter linked to a luciferase reporter gene (−109 pPC-Luc) was transfected into each of the clones. The following day the cells were treated with 0.5 mM Bt₂cAMP, or 10 uM muristerone, or both agents together as indicated in FIG. 2C. Four hours later cell lysates were prepared and luciferase activity measured as an index of transcriptional activity. Levels are shown relative to levels of luciferase activity in untreated, control cells (No Add'n). In KCREB clones 2-1 and 2-10, transcription from this promoter was efficiently stimulated 2.5 to 3.5-fold by treatment of the cells with Bt2cAMP (FIG. 2C). Prior overnight treatment of the cells with muristerone alone had no effect on basal transcription levels, but efficiently inhibited Bt2cAMP-stimulated transcription. These results are consistent with the ability of KCREB to block the activity of endogenous CREB. Alternately, muristerone treatment of VP16-CREB clones 2-4 and 9-7 stimulated luciferase production from the PEPCK promoter fragment by 3.5 to 4-fold as compared to levels measured in untreated cells. This data confirmed our hypothesis that VP16-CREB would stimulate transcription from CREB-regulated promoters in the absence of signals directed towards endogenous CREB.

Two possible mechanisms by which CREB could potentiate adipose differentiation would be by increasing cell proliferation related to clonal expansion, or by inhibiting cell growth as a prelude to terminal differentiation. To examine these possibilities, changes in the rate of cell growth in control versus muristerone-treated VP16-CREB and KCREB expressing clones was measured. No significant differences in the rate of cell proliferation were noted over a period of 72 hours following muristerone treatment between control and treated VP16-CREB or KCREB expressing cells (data not shown).

### Example 3

The following example demonstrates that CREB is necessary and sufficient to induce adipogenesis.

Based on the data above showing that CREB phosphorylation and transcriptional activity were stimulated by agents that induce adipose differentiation, it was determined whether expression of VP16-CREB would initiate or potentiate adipogenesis, and whether KCREB would inhibit differentiation in preadipocytes treated with differentiation-inducing agents. This hypothesis was first tested in experiments in which triacylglycerol storage was monitored as an index of adipose differentiation by Oil Red 0 staining.

Differentiation assays were performed on cells growing on 8 chamber microscope slides. In these experiments, NIH 3T3-L1 preadipocyte cell lines inducibly expressing VP16-CREB (clones 2-4 and 9-7) or KCREB (clones 2-1 and 2-10) or control cells (stably transfected with the plasmids, pVgRXR and piND-LacZ) were grown to confluence in high glucose medium. The cells were treated with a differentiation mixture, muristerone, or both. Cells treated with the differentiation mixture received 10 ug/ml insulin, 1 uM dexamethasone, and 0.5 mM Bt2cAMP for 48 hours, and then were refed every two days with conventional medium containing 10 ug/ml insulin. Muristerone was added to medium at a final concentration of 10 uM for the entire 10 day differentiation period. After 10 days in culture, the cells were stained with Oil Red 0 to visualize triacylglycerol vesicles as previously described (Klemm et al., 1998, J. Biol. Chem., 273:917-923), and counterstained with hematoxylin to visualize cell morphology. Cells were observed by brightfield microscopy and representative fields, were photographed. Each of the VP16-CREB and KCREB cell lines, as well as control cells (stably transfected with the pIND-LacZ expression vector) showed no signs of triacylglycerol accumulation if propagated in the absence of differentiation-inducing agents (data not shown). Likewise, all cell lines exhibited significant triacylglycerol accumulation and large, rounded morphology 10 days following exposure to a differentiation-inducing mixture of insulin, Bt₂cAMP, and dexamethasone. Thus, each of the cell lines exhibited normal differentiation characteristics. No triacylglycerol accumulation was observed in control cells treated with muristerone alone, but triacylglycerol vesicles were readily apparent in control cells exposed to both muristerone and the conventional differentiation-inducing mixture (data not shown). These results indicated that muristerone alone had no significant impact on cell phenotype.

However, in both of the VP16-CREB expressing cell lines, treatment with muristerone alone was sufficient to induce triacylglycerol accumulation and rounded cell morphology. These data indicated that VP16-CREB expression, stimulated by muristerone, was capable of initiating adipogenesis. Alternately, both KCREB expressing cell lines failed to exhibit signs of differentiation when treated with muristerone prior to and during their exposure to the conventional differentiation-inducing mixture. The ability to block adipogenesis by inhibiting endogenous CREB activity indicated that CREB is required to induce normal adipose differentiation.

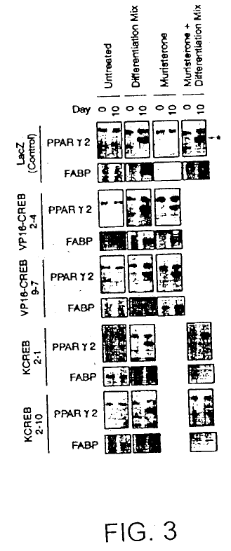

These data were confirmed by measuring the expression of the adipocyte-specific markers, PPARγ2 and Fatty Acid Binding Protein (FABP or aP2/422) in cell lysates prepared on days 0 and 10 of treatment (FIG. 3). In this experiment, control, VP16-CREB and KCREB expressing cells lines were grown and treated as above. On days 0 and 10 of the experiment, whole cell lysates and total RNA were prepared from duplicate wells of cells. Approximately 25 ug of protein in the cell lysates (lysed directly on multiwell slides in SDS gel loading buffer) was separated on 10% polyacrylamide-SDS gels and transferred to nitrocellulose blots. The blots were probed with a polyclonal antibody to PPARγ2, and the specific PPARγ2 band is indicated by an * in the top row of blots (FIG. 3).

Similarly, 10 ug of total RNA was separated on 1% denaturing (formaldehyde) agarose gels run at 5 V/cm until the Bromophenol Blue tracking dye had migrated half the length of the gel. The gels were soaked in several changes of distilled water, overnight at 4° C., and stained with ethidium bromide and briefly examined in ultraviolet light to insure RNA integrity and equivalent RNA amounts in each lane. The gels were destained in several changes of 5× sodium chloride/sodium citrate (SSC) buffer. RNA was transferred onto nitrocellulose membranes. The resulting blots were heated to 80° C. under vacuum for two hours. The membranes were blocked for 30 minutes at 70° C., and then a biotin-labeled FABP-specific oligonucleotide probe (250 fg/ml) was added for an additional 30 min. Blots were washed in three changes of 0.1×SSC, containing 1% SDS at 70° C. for 15 minutes each wash. Specific hybridization complexes were then visualized with bromo-chloro-indoyl-phosphate and nitro blue tetrazolium.

As expected, no expression of the adipocyte-specific markers, PPARγ2 and Fatty Acid Binding Protein (FABP or aP2/422), was noted in untreated control, VP16-CREB, or KCREB expressing cell lines. However, when treated with the differentiation-inducing cocktail, PPARγ2 and FABP expression were observed in day 10 samples from all cell lines. As observed for triacylglycerol storage, muristerone had no effect on the anticipated expression of PPARγ2 and FABP in uninduced and induced control cells. No PPARγ2 or FABP were present on day 0 in either VP16-CREB cell line, but were easily detected in day 10 lysates from muristerone treated cells in the absence of other differentiation-stimulating agents. On the other hand, expression of KCREB before and during the application of the conventional differentiation-inducing mixture completely blocked the appearance of both PPARγ2 and FABP in the day 10 samples. Once again, these data support the hypothesis that CREB is sufficient and necessary to initiate the adipocyte differentiation program.

A number of factors appeared to influence the ability of VP16-CREB and KCREB to regulate adipogenesis. For example, the muristerone-induced expression of VP16-CREB alone was sufficient to induce adipogenesis and lipid accumulation, as determined by spectrophotometric analysis of isopropanol extracts (Green et al., 1975, Cell, 5:19-27). However, when VP16-CREB-expressing (muristerone induced) cells were also treated with insulin, triacylglycerol accumulation was enhanced compared to cells treated with muristerone alone (data not shown). Further increases in lipid accumulation were observed in VP16-CREB expressing cells treated with insulin and dexamethasone. Although insulin and dexamethasone appeared to potentiate lipid storage in these cells, these agents did not alter the percentage of cells undergoing adipogenesis. Whether the differences in lipid accumulation reflect overall changes in differentiation related processes or simply increases in glucose uptake and/or triacylglycerol synthesis and storage has not been determined.

In similar experiments it was noted that the “complete” inhibition of adipogenesis required the constitutive expression of KCREB. When KCREB expression was induced with muristerone for only the first 48 hours of the experiment, approximately 5 to 10% of the cells exhibited low levels of triacylglycerol storage and a rounded morphology (data not shown). In contrast, no cells exhibited lipid accumulation when treated with muristerone to induce KCREB expression for the entire 9 or 10 day differentiation period (data not shown).

### Example 4

The following example shows that CREB regulates “adipocyte-specific” genes.

Without being bound by theory, the present inventors believe that CREB stimulates adipogenesis most likely through the activation of genes that drive clonal expansion (c-Fos) is and/or differentiation (PPARγ2, CEBPs α, β, and δ, and RXRs α and γ) and/or the expression of adipocyte phenotype markers (PEPCK, FABP, Fatty Acid Synthetase (FAS), Lipoprotein Lipase (LPL), Stearoyl CoA Desaturase (SCD), etc.). The promoter regions of several of these adipocyte-specific genes were visually inspected for the presence of putative CRE sequences. As shown in FIG. 4A, potential CREs present in these promoters are indicated by the box enclosed regions which surround the nucleotides which are homologous to those in the consensus CRE sequences shown at the top of the figure. FIG. 4A shows that several of these genes revealed sequences with significant homology to the consensus CRE sequence.

Next, the ability of purified, recombinant CREB to bind double stranded oligonucleotide probes of these sequences in gel retardation assays was tested as previously described in Klemm et al., 1998, J. Biol. Chem., 273:917-923. Twenty base pair, double stranded nucleotides, end labeled with y32P-ATP and polynucleotide kinase, were incubated with purified, recombinant 30 CREB protein. The reactions were separated on nondenaturing, 6% polyacrylamide gels and exposed to Kodak X-ARomat film. FIG. 4B shows a representative autoradiogram of the free (bottom) and CREB bound complexes in comparison to reactions performed with a non-specific (NS) oligonucleotide lacking a CRE sequence. Recombinant CREB was able to bind to probes corresponding to sequences in the promoters of the PEPCK, FABP, FAS, SCD, and CEBP and 6 genes, but not from the PPARγ2 or CEBP a genes. Likewise, endogenous CREB present in 3T3-L1 fibroblast nuclear extracts was shown to bind some of these promoter sequences in “supershift” gel retardation assays. In these experiments, 5 ug of nuclear extract protein prepared from 3T3-L1 fibroblasts was incubated with the indicated, labeled oligonucleotides either in the absence (−) or presence (+) of CREB specific antibody. The reactions were separated on polyacrylamide gels as described above and exposed to film. FIG. 4C shows a representative autoradiogram of unbound and protein bound oligonucleotides. Reactions containing antibody which recognizes total CREB exhibited an additional “supershifted” band that was absent in reactions lacking the CREB antibody with oligonucleotides to putative CRE sequences in the PEPCK, FABP, FAS, and CEBP β promoters. Obviously, CREB may not bind to some of the tested sequences in the context of their native promoter environment or under conditions found within the cell. Alternately, CREB may interact with the putative CRE sites we have tested in the PPARγ2 and CEBP α promoters under different conditions, or perhaps bind to other sequences in these promoters not identified by visual inspection. These data suggest that CREB may participate in adipogenesis by binding to regulatory elements in the promoters of adipocyte-specific genes.

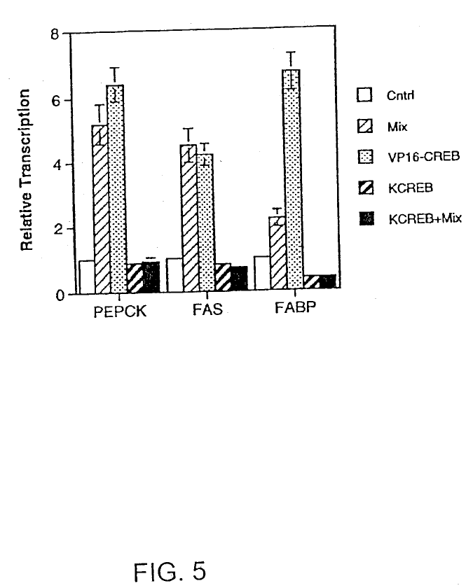

The ability of CREB to regulate transcription from three adipocyte-specific gene promoters is demonstrated in FIG. 5. Control, VP16-CREB or KCREB inducibly expressing 3T3-L1 fibroblasts were transfected with plasmids containing the full length promoters of the PEPCK, FABP or FAS genes linked to luciferase. The cells were cotransfected with the internal control plasmid, pRSV-pGal. The following day the cells were treated with muristerone to induce VP16-CREB or KCREB expression as indicated in FIG. 5, and/or with the conventional differentiation mixture of 10 ug/ml insulin, 1 uM dexamethasone and 0.5 mM Bt₂cAMP for 4 hours. Cell lysates were then prepared, and luciferase activity measured as an index of transcriptional activity. Levels of transcription are shown relative to levels measured in untreated cells for each promoter tested, and were corrected for transfection efficiency. In these experiments, transcription (luciferase production) from the “full-length” promoters of the PEPCK, FABP, and FAS genes could be stimulated by treating the cells with the conventional differentiation-inducing mixture or by induction of VP16-CREB expression. Alternately, expression of KCREB consistently decreased basal transcription levels slightly from all three promoters and completely blocked the stimulation of transcription by the differentiation-inducing mixture. Thus, CREB not only binds to CRE sequences in these gene promoters, but appears to directly modulate transcription from them.

The present inventors' data confirm that CREB and other Activating Transcription Factor (ATF)/cAMP Response Element Modulator (CREM) Inducible cAMP Early Repressor (ICER) family members play important roles in multiple cellular activities, most notably proliferation and differentiation. Initial clues to CREB's participation in these activities came from studies showing that several growth factors and other extracellular stimuli activate CREB. The present inventors demonstrated that insulin stimulates CREB phosphorylation in 3T3-L1 fibroblasts and adipocytes and HepG2 cells through an ERK½ signaling system (Klemm et al., 1998) and a decrease in nuclear PP2A activity (Reusch et al., 1995; Reusch et al., 1994). Greenberg and colleagues have reported a similar signaling cascade to CREB for NGF in neuronal cells (Ginty et al., 1994; Xing et al., 1996; Xing et al., 1998). Likewise, Fibroblast Growth Factor (Tan et al., 1996) and Insulin-like Growth Factor 1 (Pugazhenthi et al., 1998) also stimulates CREB phosphorylation and activity in neuronal cells, but this process appears to be mediated by p38 MAP kinase rather than ERK½. CREB and related proteins have also been implicated in the GI/S transition of the cell cycle in studies showing that cyclin A gene transcription is stimulated by CAMP agonists via CRE sequences in the cyclin A gene promoter (Desdouets et al., 1995).

One question to be addressed concerns the target(s) which CREB modulates in order to induce adipogenesis. The present inventors' data indicate that CREB can bind to putative CREB in the promoters of several “adipocyte-specific” genes. The binding of CREB to an oligonucleotide probe corresponding to a sequence in the CEBP β promoter was particularly interesting. CEBP β is expressed very early in adipogenesis, and will induce the differentiation of fibroblasts to adipocytes when expressed ectopically (Yeh et al., 1995). The present inventors' data suggest that one mechanism by which CREB may induce adipocyte differentiation is through an ability to stimulate CEBP β expression, which may be sufficient to induce the entire adipogenic cascade. Studies are currently underway to assess the regulation of transcription from the CEBP gene promoter by CREB, and to firmly identify CREB recognition sites in the promoter of this gene. CREB also bound to sequences from genes expressed later in adipogenesis like PEPCK and FABP. This ability suggests that CREB may play crucial roles throughout the differentiation process, or perhaps in maintaining the mature adipocyte phenotype.

With regard to how CREB regulates growth in certain cell lines and differentiation in others, one possible mechanism hinges on the availability or accessibility of proliferation-related genes in some cells and tissues versus the accessibility of differentiation-inducing genes and phenotype markers in other cell types. Applying this mechanism to adipogenesis suggests that only differentiation-inducing and/or adipocyte-specific genes rather than proliferation-inducing are accessible to CREB in preadipocytes. Another possible mechanism focuses on the interactions of CREB with other transcription factors that, in concert, exert proliferative versus differentiation-inducing effects in a cell or tissue dependent manner. Interactions between CREB and other transcription factors have been described in several systems, but their role in adipogenesis remains unclear. A number of possible mechanisms may account for CREB's participation in both proliferation and differentiation pathways. It will be interesting to decipher which mechanisms are actually functioning in these capacities, and define potential interactions between the mechanisms in the coordinate regulation of these processes.

### Example 5

The following example demonstrates a correlation between CREB content and SMC phenotype.

In light of the role of cAMP as a critical modulator of smooth muscle cell (SMC) phenotype and CREB as a modulator of differentiation in multiple tissue types, CREB distribution in bovine aortas and pulmonary arteries was investigated. Bovine pulmonary arteries and aortas were sectioned and stained with CREB antibodies as follows. Cultures of bovine SMC are assessed for contractile proteins indicative of cellular differentiation, as well as cellular content and localization of cytoskeletal and signaling proteins, by immunohistochemistry. Cells are plated on chambered microscope slides and grown to confluency. Following experimental treatment, SMC cells are fixed in ice-cold methanol. Fixed cells are treated with primary antibodies which identify α-smooth muscle actin (Sigma A2547, clone #1A4), and smooth muscle myosin (Sigma; M7786, clone LSM-V) for assessment of differentiation state, and CREB, PCREB and ICER to determine protein content. Primary antisera are removed and cells are treated with secondary antisera coupled to fluorescent compounds (FITC or Texas Red), washed, and cellular proteins visualized using fluorescence microscopy.

Vessel walls in the systemic circulation have demonstrated two morphologically and immunohistochemically distinct cell populations with highly differentiated phenotype (L2 and L3c) and contrasted with two other populations with enhanced growth potential (L1 and L3I). The results of the CREB distribution analysis showed that there is a strong correlation with high CREB content and well-differentiated high a smooth muscle actin staining in the cell populations. A very strong CREB signal was noted in the L2 and L3c areas whereas staining was light in the L1 and the L3I areas (data not shown). These data suggest that CREB content is either a determinant of or a marker for SMC differentiation. Additionally, decreased CREB content was noted in areas with high growth potential. This population has been implicated in restenosis and atherosclerosis where an exaggerated SMC proliferative response is seen.

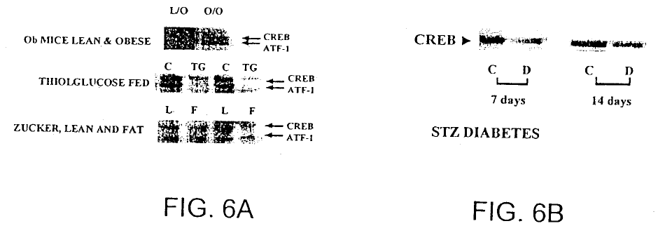

It is clear clinically that diabetes and insulin resistance are associated with increased atherosclerosis. A number of groups have described increased restenosis and enhanced SMC phenotypic modulation in animal models of these diseases. In preliminary studies, total aorta CREB content in animal models of insulin resistance and diabetes was assessed. Homogenates from aorta tissue from three rodent models of insulin resistance (ob/ob and lean/ob mice, lean mice and thiolglucose-injected corresponding littermates, and lean and obese Zucker rats) were analyzed by SDS-PAGE electrophoresis. 30 ug of homogenate were loaded on 12% gels and analyzed for CREB protein content by immunoblot. FIG. 6A shows that insulin resistance was associated with a decreased vascular wall CREB content. Thoracic aortas harvested from STZ rats also demonstrate a clear decrease in aortic CREB protein content at 7 and 14 days of diabetes compared to controls (FIG. 6B). Preliminary analysis of scanning densitometry revealed a 49% decrease in the ob/ob, 51% decrease in thiolglucose fed, 38% decrease in Zucker (F) and 40% decrease in STZ animals.

### Example 6

The following example shows that cAMP is a critical determinant of SMC phenotype.

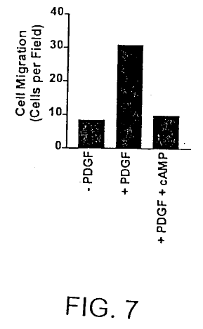

To confirm literature reports of the importance of cAMP for the smooth muscle cell (SMC) phenotype, bovine aorta smooth muscle cells (BASMC) were examined for changes in morphology and migration upon modulation of PKA activity. These cells are from a single vascular bed and harvested from an ingrown strain of cattle. BASMC are an established model of vascular smooth muscle tissue in culture (Cucina et al., 1999, J. Surg. Res., 82:61-66; Frid et al., 1997, Circ. Res., 81:940-952; Frid et al., Arterioscler Throb Vasc Biol 17:1203-1209,1997; Hatzi et al., 1999, Eur. J. Biochem., 260:825-832; Stenmark et al., Chest 114:82S-90S, 1998; Takahashi et al., Circ Res 84:543-550, 1999; Underwood et al., 1998, J. Vasc. Res., 35:449-460), and are useful for assessing vascular smooth muscle cell proliferation, migration and contraction. Inhibition of PKA activity by administration of 10 uM H89, a PKA inhibitor, for 4, 8 and 24 hours, resulted in significant alterations in cellular morphology relative to untreated control cells (data not shown). Specifically, H89 results in loss of SMC “spindle shape” representative of differentiated, contractile phenotype. Treatment of BASMC with 0-10 uM H89 for 72 hours decreased α SM actin content and led to structural rearrangement of contractile fibers. These results demonstrate a clear alteration in SMC morphology in cells treated with the PKA inhibitor H89. As shown in FIG. 7 (values represent the mean of 8 fields per Boyden Chamber membrane), treatment of BASMC with 0.5 uM dibutyrl cyclic AMP, a stimulator of PKA, blocked PDGF stimulated SMC migration (100 pM PDGF). These data support the importance of PKA for SMC phenotype and illustrate the ability to detect differences in morphology and migration in SMC.

### Example 7

The following example shows that high glucose and high insulin induce CREB phosphorylation and decrease CREB content.

It has been demonstrated in the literature that both hyperinsulinemia (HI) and hyperglycemia (HG) cause SMC phenotypic modulation (Absher et al., Atheroscerosis 143:245-251, 1999; Avena et al., J Nasc Surg 28:1038-1039, 1998; Kimura et al., Immunopharmacology 40:105-118, 1998; Wang et al., Nippon Ika Daigaku Zasshi 65:284-290, 1998). Studies were undertaken in BASMC to examine the impact of high glucose (HG) and/or high insulin (HI) on CREB and ICER content. BASMC were grown to confluence, ss for 24 hours, then treated with the concentrations of insulin and/or glucose. HI exposure for 72 hours increased CREB phosphorylation with a minor decrease in CREB content (−24%±7 n=4; data not shown). Exposure of BASMC to hyperglycemia leads to delayed increases in PCREB with significantly decreased CREB protein content (−32%±6 n=6 p<0.05) at 72 hours (data not shown).

Next, BASMCs were exposed to either 3 nM or 100 nM insulin for 72 hours and analyzed for mRNA content of ICER. ICER protein was induced by physiological doses of glucose and insulin. Supraphysiological doses of glucose induced ICER protein without insulin. High dose insulin decreased ICER content at the 72 hour time point and increased the presence of ICER degradation products (not shown). BASMC were cultured to 90% confluence and treated with glucose (5 and 25 mM) and insulin (3 and 100 mM) for 72 h. Total RNA was isolated from these cells using Qiagen's RNeasy kit. RNA samples were fractionated on denaturing 1.2% agarose-formaldehyde gels and transferred to Hybond N+ membrane. ICER IIγ CDNA probe was labeled with thermostable alkaline phosphatase using the AlkPhos-Direct kit from Amersham Lifesciences (Buckinghamshire, England). Hybridization, washing and detection by CDP-Star were performed according to manufacturer's protocol. The results demonstrated that high PCREB is capable of inducing ICER. Examination of ICER in cells treated with either insulin and/or glucose demonstrated an increase in ICER mRNA. This data supports a direct role for glucose in CREB down-regulation noted in STZ-DM and suggests that insulin disrupts CREB signaling via ICER induction.

Taken together these data support hypothesis that hyperglycemia and hyperinsulinemia can be associated with changes in CREB, ICER or both. ICER induction is most prominent at 24-48 hours and falls off as CREB content declines around 72-96 hours.

### Example 8

The following example demonstrates transfection and infection of BASMC with CREB constructs, and the ability of such constructs to alter SMC phenotype.

In order to delineate the importance of CREB for SMC phenotype and post-angioplasty restenosis, it is critical to be able to transfect or infect cells with CREB reporter constructs, specific genes of interest or modulators of CREB function. To this end, a series of experiments were done to determine the feasibility of gene transfer in BASMC.

Early passage cultures (P1-P5) of SMC cells were plated in 6-well culture dishes at a density of 1.4×10⁵ cells/cm² and maintained in Growth Medium for 18 hrs. Transfection was performed using Lipofectamine Plus Transfection Reagent (GIBCO/BRL) as described by the manufacturer. In addition to specific chimeric promoter-luciferase plasmid constructs, SMC cells were cotransfected with a constitutively-expressed beta-galactosidase reporter plasmid construct (RSV-βgal). The Lipofectamine Plus:DNA mixture in Growth medium was left on cells for 3 hrs, and allowed to recover in Growth Medium overnight. SMC cells were subsequently serum-starved in 1×MEM (Minimal Essential Medium containing 1× Non-Essential Amino Acids and 0.4 mM glutamine) for 24 hrs. Agonist treatment was performed in 1×MEM for duration's of 4-24 hrs, and cells were subsequently extracted in 1× Reporter Lysis Buffer (Promega) for analysis of reporter gene expression. Luciferase Reporter activity was corrected for differences in transfection efficiency, cell number, and extract recovery, using beta-galactosidase activity determined in the same cellular extract. Results showed a 5-15% efficiency in transfections using lipofectamine (data not shown).

In initial adenoviral delivery experiments, SMC were transfected to express Green Fluorescent Protein (GFP) using recombinant, replication-deficient adenovirus under the regulation of the CMV immediate early promoter (i.e., a viral vector comprising SEQ ID NO:19). BASMCs were plated at a density of 1.4×10⁵ cells/cm² in 24 well culture dishes and maintained in Growth Medium for 24 hrs. Cultures of BASMCs were treated with recombinant replication-deficient adenovirus at a titer of 100-1000 particles per cell for 4-24 hrs. Transfection efficiency, as indicated by GFP expression, and SMC cell viability were assessed in adenovirus-treated cultures 24-48 hrs post-infection. Expression of GFP was determined by fluorescence microscopy, while cell viability was crudely determined by microscopic assessment of cell density and morphology. Results showed a 90-100% infection with adenovirus without appreciably altering morphology (data not shown).

The observation that CREB content was high in SMC with highly differentiated phenotype suggested that CREB could be playing a role in causing that phenotype. To test the validity of that hypothesis the present inventors introduced CREB into SMC using transfection and also using adenoviral infection. These studies demonstrated that overexpression of CREB and constitutively active CREB decrease mitogen and glucose stimulated migration and proliferation.

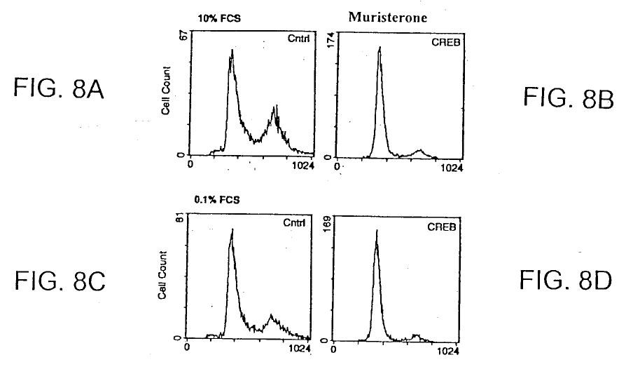

BASMC were stably transfected with the muristerone-inducible wild-type CREB (WT CREB) expression vector. They were then exposed to serum at 0.1 or 10% with and with exposure to muristerone to induce CREB expression. FIGS. 8A-8D show control cells on the left and muristerone cells on the right. Cells were incubated with Krishan's stain and subjected to fluorescence flow cytometry to assess proliferative capacity to determine phase in the cell cycle. The cells treated with 10% FCS showed a significant portion in S phase, second peak, indicating active DNA replication consistent with proliferation. This peak was smaller in the cells incubated in low serum. Induction of WT CREB expression with muristerone led to cell cycle arrest with the majority of the cells appearing in the Go peak. These data are consistent with the hypothesis that increased CREB protein content can modulate proliferative capacity. The stably transfected clone served as its own control. Preliminary studies were next undertaken to investigate the impact of adenoviral infection with constitutively active CREB (adVP16 CREB) on CRE driven gene expression, proliferation, and migration.

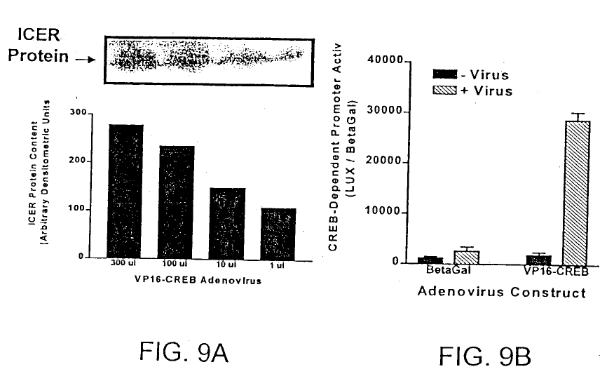

SMC are difficult to transfect with high efficiency and tend to change phenotype with multiple passages. It therefore seemed likely that a highly efficient adenoviral infection strategy could most effectively and efficiently address the present inventors' questions regarding phenotype. The first in a series of recombinant CREB adenoviral constructs to be created was the constitutively active isoform VP16CREB (adVP16CREB). This construct infects SMC with high efficiency. To test the functional consequences of infection with this construct, its ability to drive transcription of an exogenous CREB-dependent promoter-reporter construct (CREluc), as well as its ability to induce CREB-dependent ICER gene expression in SMC, was examined. First, SMC infected with 0-300 ul of crude adVP16CREB were lysed and assessed for protein content of the CREB dependent gene ICER. FIG. 9A shows that ICER content increased with increasing doses of adVP16CREB. A representative immunoblot of one of three such experiments is shown at the top of panel FIG. 9A.

SMC transiently transfected with a CREluc reporter construct (STRATAGENE) were infected with 300 μl of crude adVP16CREB and assessed for reporter activation. FIG. 9B shows that adVP16CREB infection led to a significant (30-61 fold) increase in luciferase activity relative to that seen with adBetaGal.

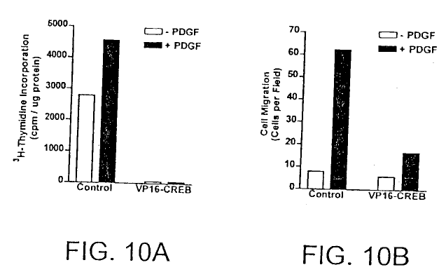

It was next important to assess the impact of adVP16CREB infection on SMC phenotype. BASMC were grown to 70% confluence and serum starved for 48 hours. Cells were then treated with 0.1 uM PDGF and assessed for thymidine incorporation (FIG. 10A) and migration (FIG. 10B) as follows. Rates of DNA synthesis in cultured SMC cells were estimated by determination of the rate of incorporation of ³H-thymidine into cellular DNA. SMC cells plated in 12 well plates were subjected to experimental treatment, and pulsed with ³H-thymidine (2.0 μCi/ml) 21 hr post-treatment. Cell counts were made at the onset of the experiment to ensure that equivalent numbers of cells are present in each experimental sample. ³H-thymidine incorporation into SMC cell DNA is expressed as disintegrations per minute (DPM) per cell and DPM per well. Migratory behavior of SMC cells was assessed using a 12 well micro-Boyden chamber apparatus (Transwell Apparatus, Costar, Corning, N.Y.). Chemotactic compounds, such as Platelet-derived Growth Factor-BB, were diluted in serum-free medium and added to the lower chamber of the apparatus. Wells were covered with a Type I collagen-coated PVP-free filter with 8 mm pores (Transwell Apparatus, Costar, Corning, N.Y.). Trypsinized cells were resuspended in serum-free medium containing either chemotactic compounds or vehicle, and plated at a density of 25,000 or 50,000 cells per well in a volume of 100 μL. At the end of the incubation, cells attached to the filter were fixed and stained in Dif Quick (American flospital Supply Corp., McGraw Park, Ill.). Cells that have migrated, located on the underside of the filter, were counted manually and migration expressed relative to control.

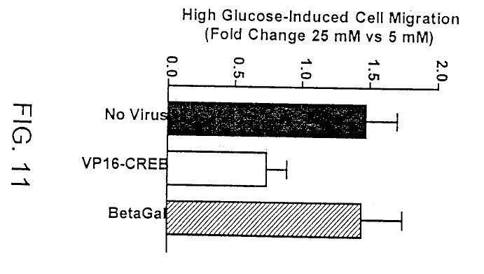

FIGS. 10A and 10B are representative of three similar experiments done in triplicate. FIGS. 10A and 10B show that adVP16CREB dramatically decreased PDGF-stimulated thymidine incorporation and cell migration in SMC. Moreover, treatment of SMC with high glucose (25 mM vs 5 mM) for 48 h resulted in increased cell migration in Boyden Chamber experiments. FIG. 11 shows that infection with adVP16CREB (but not adBetaGal) attenuates glucose-induced acceleration of cell migration (n=6 each group). These studies clearly demonstrate the ability of constitutively active CREB to promote a more highly differentiated phenotype.

### Example 9

The following example shows the effect of various agents on CREB expression and activity.

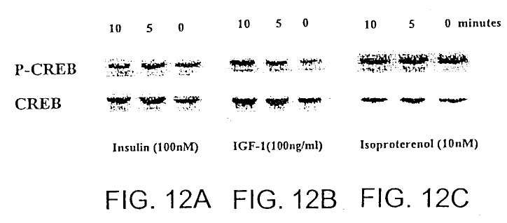

CREB drives transcription when acutely phosphorylated on serine 133. A series of experiments was performed to determine the ability of insulin, IGF-1 and ISO to acutely enhance CREB phosphorylation in BASMC. BASMC were grown to confluence, ss for 48 hours, and acutely treated with insulin, IGF-1, or Iso in the concentrations noted in FIG. 12A-12C. FIGS. 12A-12C shows that each of these agents acutely increases P-CREB/CREB content.

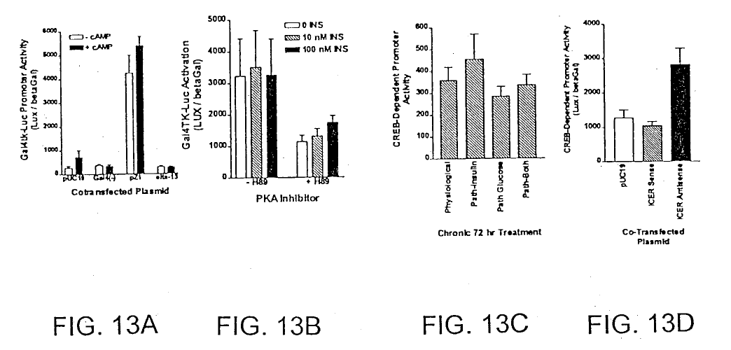

Isoproterenol is well documented to act through cAMP, yet no studies defining its impact on CREB transactivation in SMCs have been reported. In the next experiment, the ability of ISO to drive a CREB dependent reporter construct was determined. BASMC were transfected with Gal4-TKluc plasmid containing the Gal4 promoter and a thymidine kinase enhancer upstream of the gene encoding a luciferase reporter. These cells were cotransfected with control plasmid (pUC19), a plasmid encoding the Gal4 protein alone, or plasmids encoding chimeric Gal4 proteins with either wild type CRF-B (pZI-Gal4-TKluc) or Ser 133 mutated CREB (SeYI33). FIG. 13A shows that only the wild type CREB construct activated CREB-dependent Gal4-mediated luciferase production, and mutation of the Ser 133 residue ablates endogenous CREB kinase mediated transcription. FIG. 13B shows that treatment of BASMC with the PKA inhibitor H89 attenuates high endogenous CREB kinase activity and unmasks insulin-sensitive CREB transactivating potential. Of note, basal PKA mediated CRE reporter transcription is very high in these cells and is blocked by the PKA inhibitor H89. Inhibition of PKA with H89 reveals clear dose-dependent transcriptional activation of CREB by insulin in these cells. FIG. 13C shows that treatment of BASMC with pathological concentrations of insulin (100 nM) increases CREB-dependent promoter transactivation, while pathological glucose (25 mM) attenuates this activity. FIG. 13D shows that transfection with expression vectors containing the cDNA for ICER II gamma in the sense or antisense orientation modulate CREB-dependent promoter activity. Expression of the ICER-sense construct diminishes CREB-mediated transcription slightly, while transfection with the ICER-antisense construct results in a significant increase in CREB-mediated transcription. These results are consistent with ICER's role as a negative regulator of CREB activity, and also with the presence of a high level of endogenously expressed ICER protein in cultured BASMC. Taken together, these studies demonstrate the ability to transfect BASMC and get adequate expression of reporter constructs and also show that the ICER constructs function as anticipated.

### Example 10

The following example shows that adenoviral vectors encoding CREB constructs of the present invention regulate CREB dependent transcription.

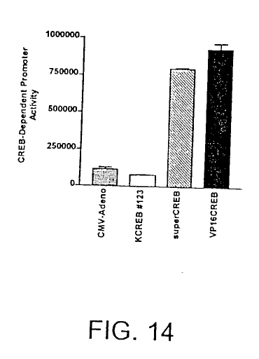

CDNA for KCREB, CREB DIEDML and VP16CREB were cloned into adenoviral expression vectors as described previously herein. The plasmids were transiently transfected into HEK cells to assess their impact upon a cotransfected CREluc reporter construct. FIG. 14 shows that VP16CREB and CREB DIEDML strongly drive the reporter whereas KCREB decreases luciferase expression, indicating that the constructs are functioning as expected. Transfected cells were also lysed and CREB protein content and Flag content assayed Western analysis. All proteins were highly expressed in HEK cells (data not shown).

### Example 11

The following example demonstrates detection of post-angioplasty neointimal thickening in rat carotid arteries in an animal model for investigating modulation of cell differentiation using CREB constructs.

For the detection of changes in post-angioplasty intimal thickening it is necessary to be able to induce a clear intimal thickening in response to balloon injury. Rat carotid arteries were subjected to balloon angioplasty. Briefly, 16 week old Sprague Dawley rats were anesthetized, the ventral neck will be clipped and scrubbed with antiseptic agent (betadine). Using aseptic techniques, a ventral midline skin incision was made and sharp and blunt dissection was used to expose the animal's left carotid artery. Sutures were loosely placed around the left common carotid artery and external carotid artery for traction to minimize blood loss during the angioplasty procedure. A ligature was placed at the cranial-most aspect of the external carotid arteries. A small incision was made using ophthalmic iris scissors, and a cannula made of polyethylene tubing (PE-160) was introduced into the incision. A #2F balloon embolectomy catheter was introduced into the external carotid artery and pushed caudally through the common carotid artery until the aortic arch is reached. The catheter's balloon was then inflated with sterile saline and pulled cranially until the balloon reaches the external carotid artery. At that point, the balloon was deflated and the catheter pushed caudally to the aortic arch again, and the process repeated for a total of three passes of the inflated balloon up the common carotid artery. After the third pass, the balloon catheter was removed and the external carotid artery will be ligated caudal to the arterial incision (just cranial to the bifurcation of the carotid artery). The area was inspected for hemorrhage and the incision closed with 3-0 nylon skin sutures. The animals' recovery was monitored postoperatively. They were kept warm by placing cages under a heat lamp, and the rats were given intraperitoneal isotonic fluids if the surgery took excessive time or the animals appeared to be having a delayed recovery. After recovery, the rats were housed in animal care facilities until they were euthanized.

Carotid arteries subjected to angioplasty, as well as their sham-contralateral controls, were removed 14 days after the procedures, sectioned, and assessed histologically for the development of neointima. Histological assessment of the intimal thickening demonstrated a clear quantitative increase in neointimal cells in balloon injured carotid arteries (data not shown).

### Example 12

The following examples demonstrates that augmentation of CREB protein expression by adenoviral gene transfer at the time of angioplasty will promote SMC differentiation and thereby decrease post-angioplasty restenosis.

Optimal doses for adenoviral delivery will be established, and animals undergoing balloon angioplasty will be infected with adenoviral control, wild type CREB (WT-CREB), or one of two different types of constitutively active CREB, VP16CREB or CREB DIEDML at the time of balloon injury (described in Example 11above). The VP16CREB is a chimeric CREB protein that contains the DNA binding domain of CREB fused to the transcriptional activation domain of the viral oncogene VP16. This fusion protein has a strong capacity to activate CRE dependent transcription. The CREB DIEDML construct was provided by Richard Goodman (Vollum Institute, Portland Oreg.). CREB DIEDML is a full length CREB with a series of mutations that leads to constitutive binding to CREB binding protein (CBP). CBP interacts with the transcriptional machinery to activate CRE dependent transcription.

Initial studies to determine the appropriate viral load and infection efficiency will be done using adenoviral green fluorescent protein (GFP) and adenoviral beta galactosidase (b gal) already available in the laboratory. The gene delivery will be conducted generally as described by Rodman et al., showing a series of experiments using adenoviral gene vectors delivered intra-arterially (Rodman et al., Am J Respir Cell Mol Biol 16:640-649, 1997; Varenne et al., Circulation 98:919-926, 1998). For the in vivo viral infection studies, adult male rats will undergo carotid artery balloon catheter injury as described above, except using a double balloon catheter. The arteries will be injured with a double balloon catheter and then infected with a sterile, replication-incompetent adenovirus construct for 10-20 min prior to incision. The specific details of the anesthesia, duration of balloon injury, calculation of viral load and peri-operative animal handling will be determined experimentally. To collect neointimal SMC for cell culture, the animals will be euthanized at the post-injury intervals detailed under experimental design and carotid arteries will be removed. The carotid arterial media and neointima will be aseptically dissected and SMC cultures will be started by explant techniques. Neointimal SMC will be selected based on their capacity to grow from explant within 24 hours (as described by Weiser-Evans et al., 1999, J. Vasc. Sturg. 29:1104-1151, abstract). Cells will be maintained in 10% calf serum in DMEM and will be used for experiments in the 2nd to 4^{th} passages.

The specific study design is as follows:

| Treatment | Right Carotid | Left Carotid |
|---|---|---|
| Control | Injury | Control |
| Viral control | Injury adenoGFP | Control adenoGFP |
| WT CREB | Injury adWT CREB | Injury adVECTOR |
| WT CREB | Injury adWT CREB | Control adWT CREB |
| VP16 CREB | Injury VP16 CREB | Injury adVECTOR |
| VP16 CREB | Injury VP16 CREB | Control VP16 CREB |
| CREB DIEDML | Injury CREB DIEDML | Injury adVECTOR |
| CREB DIEDML | Injury CREB DIEDML | Control CREB DIEDML |

6 animals per group per time point

Dose ranging studies will be done on both control and balloon injured vessels to determine optimal dose and volume, as well as ligation tire. For these studies a minimum of 6 animals will be examined per group to permit statistical analysis. For control purposes, the animal will be treated using the opposite carotid as a control. Two types of control are Is critical. First, for comparison of the injury response the animal will need to be lesioned bilaterally and treated on one side with CREB and on the other with vector alone. The second control will be to treat an injured vessel with CREB and compare that to the uninjured CREB treated vessel. Animals will be euthanized at 7 days post-balloon injury and their carotid arteries harvested for histological assessment of intimal thickening, viral expression, apoptosis markers, and DNA replication. Neointimal cells and medial cells will be harvested at similar time points and placed in primary culture for evaluation of CREB protein content and functional studies to define proliferation, migration and CREB dependent gene expression (assessed as described in examples above and in addition methods below).

These studies will permit evaluation of the impact of functional CREB on post-injury intimal thickening. Without being bound by theory, the present inventors expect that WT CREB and CREB DIEDML will decrease neointimal thickening in response to balloon injury. VP-16 CREB is expected to initially blunt the response as well but the extremely high transactivation seen with this construct could lead to either increased cell survival (which could result in intimal thickening). It could also result in inadequate post-injury repair because it take the cells out of cell cycle so effectively. The most informative experiment will be with the WT CREB as it is the protein depleted in highly proliferative regions of SMC and animal models known to have exaggerated restenosis. Seven days will be the main time point, and later, 14 and 28 day timepoints will be assessed as the lesions mature to assess the longer-term consequences of CREB overexpression.

Additional Methods:

Cell proliferation: Alterations in cell number in response to experimental interventions are determined by counting cells using standard hemocytometry. Cells are trypsinized for 5 min, gently titrated in an equal volume of Growth Medium, and an aliquot subjected to counting. Late cellular proliferation is assessed through a combination of cell counting and MTT assay (see below).

MTT assay for Late Cellular Proliferation: 3-(dimentylthiazol-2-yl)-2,5-diphenyl tetrazolium bromide (MTT) is taken up and converted by viable cells to an insoluble blue compound by mitochondrial activity. RASMCs are plated at 2,000 cells per well in a 96 well dish and subjected to experimental treatments. At the end of the experimental treatment, 20 μL of MTT solution (5 mg/ml in Phosphate Buffered Saline; PBS) is added to each 200 μL well and incubated at 37° C. for 4-6 hrs. Plates are spun in an IE Centrifuge for 3 min at 800 rpm and the culture supernatant is carefully removed from the well: 150 μL of acidic isopropanol (70% isopropanol, 30% 0.2 N HCl) is added to each well, and blue MTT-derivative is dissolved overnight at 4° C. Blue derivative is quantified spectrophotometrically by measuring absorbence on an ELISA plate reader using 550 nm and 590 nm filters.

Histological Analysis: Tissue sections of carotid arteries will be processed as described by (Frid et al., Circ Res 81:940-952,1997; O'Brien et al., Circulation 98:519-527, 1998). Bilateral rat carotids will be harvested and histological sections will be stained for CREB, PCREB, ICER, 4′,6′-diamino-2-phenylindole (DAPI), and microfilaments, as well as the candidate target genes. Histological quantification of protein distribution will be scored using the optima image analyzer and evaluating 10 HP fields per section as described by O'Brien (O'Brien et al., Circulation 98:519-527,1998). For histological analysis of lesion size and intimal thickening, tissue sections will be stained with hematoxylin and eosin. The cross-sectional area of neointima will be measured on each vessel using the NIH Image program (analyses will be performed on a Macintosh computer using the public domain NIH Image program; developed at the U.S. National Institutes of Health and available on the Internet at http://rsb.info.nih.gov/nih-image/). Medial area will be defined as the area enclosed by the internal and external elastic laminae, whereas intimal area will be defined as the area between the internal elastic lamina and the perimeter of the lumen.

Flow cytometry: Alternatively, the cellular content of specific proteins or the number of cells expressing a particular protein is assessed using fluorescence flow cytometry. Cells are trypsinized and fixed in culture medium containing 4% formalin for 30 min. Cells are rinsed in PBS, permeabilized with ice-cold ethanol for 10 min, washed, centrifuged, and resuspended (2-3×10⁴ cells/ml) in 3% FCS in PBS to quench nonspecific antibody binding. 3-5×10⁵ cells are transferred to 96 well dishes for immunostaining. Cells are incubated in the presence of the appropriate antisera at 4° C. for 30 min, washed twice, and incubated in fluorescein isothiocyanate-coupled secondary antibody for 30 min at 4° C. Cells are washed and resuspended in PBS and subjected to fluorescence flow cytometry (Coulter Epics Profile) using an argon laser at 488 nm. Cellular content of specific proteins is expressed in arbitrary fluorescence units per cell or the proportion of fluorescent cells.

In Situ Hybridization: Sections of rat carotid artery, either those subjected to angioplasty and it's contralateral control, will be examined for alterations in CREB and ICER mRNA content using in situ hybridization. Sections from coronary arteries removed from experimental animals will be fixed in 4% paraformaldehyde-lysine periodate fixative for 24 h, followed by incubation in 70% ethanol. Sections are rehydrated in aqueous ethanol of decreasing ethanol concentrations prepared in RNase-free water. All procedures after this point utilize RNase-free water.

Sections are pretreated in 0.025 N HCl for 10 min, rinsed in RNase-free water, treated with Proteinase K (20 mg/ml), and rinsed with water. Sections are dehydrated in aqueous ethanol increasing ethanol concentrations air dried, and outlined with hydrophobic isolator. Sections are prehybridized against 100 μL of Amresco prehybridization buffer (Amresco, Cat #0973) for 2 h at 42° C. Single-stranded RNA riboprobe, produced from plasmids containing cDNA encoding CREB or ICER, labeled with digoxigenin-coupled UTP (Boehringer Mannheim), is added to prehybridization solution and samples incubated overnight at 42° C. in a humid chamber.

Slides are subjected to increased stringency washes (2×SSC to 0.5×SSC), 45 min each at room temperature in RNase-free glassware. A single wash with 20 mg/ml RNase in 0.5×SSC is performed at 37° C. to remove non-specifically-bound single-stranded probe, followed by two washes in 0.5×SSC. Following blocking in 2% sheep serum containing 0.3% Triton X-100, probes are detected using diluted alkaline phosphatase-conjugated digoxigenin-antibody for 2 h at room temperature. Slides are washed for 10 min each in TRIS-based buffers, and color solution (containing NBT/BCIP and levamisole) added to each section. Sections are incubated overnight in the dark. The color reaction is stopped, and sections counter stained in Fast Green for 1 min. Slides are mounted and sections examined by light microscopy.

### Example 12

The following example shows whether the impact of CREB content restoration on post-angioplasty restenosis influenced by diabetes or insulin resistance.

Diabetes and insulin resistance are associated with a higher incidence of restenosis. CREB protein content is lower in vessel walls of animals with diabetes and insulin resistance. These studies will examine the impact of changing functional CREB content on restenosis in animal models of diabetes and insulin resistance.

Parallel questions to those asked in Example 11 will be asked in animals with STZ-DM. If these studies are informative, an additional series of animals with genetic insulin resistance, Zucker rats, will be examined. The studies will be carried out exactly as detailed in Example 11, with one important exception: only disease modifying CREB isoforms will be examined.

| 7 day STZ DM animals | Right Carotid | Left Carotid |
|---|---|---|
| Control | Injury | Control |
| Viral control | Injury adenoGFP | Control adenoGFP |
| WT CREB* | Injury adWT CREB | Injury adVECTOR |
| WT CREB* | Injury adWT CREB | Control adWT CREB |

*Other CREB isoforms will be examined as indicated from studies above

It is expected that intravascular CREB protein content will be low at baseline in these animals. Thus, introduction of WT CREB is expected to be more efficacious in these experiments than in control animals. In the injured vessels, we expect that WT CREB, VP16 CREB and CREB DIEDML will decrease intimal thickening.

The use of animals with either chemically induced DM or genetic insulin resistance adds a level of complexity for the interpretation of the results. Each of these conditions is associated with changes in SMC function as well as decreased CREB protein content. For these reasons it will be essential to examine the control carotid for disease related changes. All studies will be analyzed in the context of control animal balloon injury as well as in comparison to the non-injured vessel of the diseased animal. If CREB plays a role in restenosis, it could be more important in these animals where CREB is depleted prior to balloon injury.

### Example 13

The following example shows whether CREB content or function is altered following balloon injury.

Altering smooth muscle cell CREB protein content with an inducible CREB expression vector decreases entry into cell cycle. Infecting SMC with an adenoviral construct containing a constitutively active CREB decreases thymidine incorporation and migration. Post-angioplasty, neointimal SMC are highly proliferative and migratory when compared to medial SMC. This is strikingly similar to the differences between the L1 (poorly differentiated) and the L2 (highly differentiated) cell populations the present inventors have previously characterized. It is important to determine whether CREB content or function is altered in the post-angioplasty model.

These studies will characterize neointimal versus medial SMC for CREB content, CREB transcriptional activation capacity, content of the negative CREB modulator, Inducible CREB Early Repressor (ICER), as well as functional properties such as migration, cell cycle and proliferation. SMC cultures will be started by explant techniques from injured and control vessels. Neointimal (NEO) and medial (MED) SMC will be collected from control and injured vessels at 0, 4, 7, 14 and 28 days. This series of time points is important to detect the transient changes in SMC phenotype that occur early after angioplasty and return to a more quiescent phenotype by day 14-28 post-injury. As mentioned in the background section cyclic nucleotides decrease in the post-angioplasty vessel and are restored to control levels between 14 and 28 days. The impact of balloon injury on CREB has not been characterized or reported in the literature. Passage one explanted cells will be assayed for CREB and ICER by Western analysis. Migration, proliferation and cell cycle will be determined as described in previous examples. Very few cells are required for these assays so it is feasible to use passage 1-2 cells. Transcriptional activity will be assessed by two strategies: transient transfection (initially) and later adenoviral infection with an adenoCREluc (when the constructs are completed). To obtain adequate cell numbers for these studies it will be necessary go to higher passage (3-6). Initial studies will be done only in control animals not exposed to either diabetes or in vivo gene transfer. If these studies are informative, the STZ DM animal time course and infected animals will be examined.

It is expected that in the control animals the NEO cells will have low CREB content and function, especially at the 4 and 7 day time points. These cells should exhibit increased proliferation and migration. At 14 and 28 days the cells should become more differentiated and we expect CREB content to return to baseline. This phenotype should be exaggerated by the existence of diabetes or insulin resistance. These studies will provide background for mechanistic experiments to examine CREB content with time in diabetic animals. It will also enable the determination of the most critical time point post-angioplasty for examination of CREB dependent target genes.

### Example 14

The following example demonstrates whether a correlation exists between CREB content and CREB dependent gene expression in post-angioplasty vessels.

A significant number of genes known to be dysregulated in diabetes and atherosclerosis contain CREB binding sequences (CREs) in their promoter regions. Examples of these include VEGF, PAI-1, and tPA. Alteration in CREB content could affect the regulation of these CREB targets. These studies will examine the expression of mRNA and protein of these candidate CREB target genes and correlate this expression with vessel wall CREB and ICER content. Additional targets will be identified using array analysis.

Initial experiments, using tissues harvested in Example 11, will examine the expression of ICER, VEGF, PAI-1, and tPA by in situ hybridization, immunohistochemistry and Western blot analysis. These findings will be correlated with CREB protein content. Cells isolated from control and post-injury vessels will be cultured and transiently transfected with promoter reporter constructs for ICER, VEGF, PAI-1, and tPA, to assess whether a difference in basal expression exists between control and injured vessels. Multiple agents and signaling events are likely to be responsible for alterations in expression of these candidate genes. To assess the importance of CREB for any changes observed, two strategies will be employed. First, the impact of adenoviral infection with control, WT-CREB, VP16CREB and CREB DIEDML on the mRNA content of ICER, VEGF, PAI-1, and tPA will be assessed. Additional studies will examine the impact of infection with dominant negative CREB isoforms K-CREB and CREB M1 on these genes. Second, dominant negative and constitutively active CREB expression vectors will be cotransfected with the promoter reporter constructs for ICER, VEGF, PAI-1, and tPA.

It is entirely possible that while the above mentioned target genes contain numerous CREs, CREB may not be a primary determinant of their content. With this in mind, parallel studies will be undertaken to contrast the gene expression pattern between NEO and MED cells using array analysis. It will then be of interest to infect the NEO cells with WT-CREB, K-CREB, CREB M1, VP16CREB and CREB DIEDML and compare them with the parental cell lines. These infections will be done in explanted NEO and MED cells rather than cells from infected animals so adequate amounts of RNA can be isolated.

These experiments are straightforward and will yield important new information regardless of the impact of CREB on these genes. It is expected that induction of VEGF and PAI-1 expression and decreased expression of ICER and tPA in NEO SMC will occur. The importance of CREB and ICER for the expression of VEGF, PAI-1, and tPA is unknown as these are candidate genes and not proven CREB downstream targets. For this reason a second strategy of differential array analysis will also be examined. This will surely identify candidate structural, cell cycle, and transcriptional genes worth exploring more fully.

Array analysis: The experiments described above will determine if expression of CREB can influence angioplasty-induced changes in SMC biology, specifically proliferation and cell migration. The genetic changes which underlie these alterations in SMC biology will be examined using Atlas cDNA Expression Array Analysis (Clontech). Differences in the patterns of gene expression will be examined (1) in neointimal and medial SMC following angioplasty, and (2) in SMC prior to and following exogenous expression of CREB resulting from recombinant adenovirus-mediated gene transfer. Briefly, total RNA will be extracted from SMC using the guanidinium isothiocyanate:phenol:chloroform method. Poly A⁺ RNA (mRNA) will be isolated by oligo-dT-affinity chromatography. cDNA will be synthesized from SMC mRNA, and labeled as recommended by Clontech. CDNA probes produced from SMC mRNA from different experimental groups will be hybridized to separate Atlas CDNA Array membranes. Differences observed in the intensities of signals for given “spots” on comparable membranes represent differences in the abundance of specific mRNA between experimental groups. As such, differences in the patterns of SMC expression of 588 different genes, which are the result of either the location of SMC in the vessel wall or of exogenous expression of CREB, can be rapidly assessed.

### Example 15

The following example shows the impact of chronic hyperglycemia and hyperinsulinemia on CREB content and phosphorylation state, and on the expression of ICER in SMC.

For the initial studies, primary cultures of bovine aortic vascular smooth muscle cells (BASMC) are used. Once each of the assays is optimized, a parallel series of experiments are done in SD rat aortic SMC (RASMC). The in vitro experiments under control HG, HI conditions employ passage 3-5 RASMC. For examination of control-vs-STZ-DM and or IR animals passage one cells will be used to avoid obscuring of differences secondary to passaging in culture.

It has been proposed that cAMP acts as a gate for mitogenic signaling by inhibiting ERK MAPK activation and inducing cell cycle arrest (Graves et al., 1993, Proc. Natl. Acad. Sci. USA, 90:10300-10304; Iyengar, Science 271:461-463, 1996). Protracted agonist stimulation, which chronically increases cAMP content, blunts further signaling through CREB by decreasing CREB content and attenuating transcriptional responses to acute stimuli. This has been demonstrated in the setting of β adrenergic stimulation induced cardiomyopathy. One consequence of sustained or high magnitude CREB phosphorylation is the induction of ICER which can inhibit CREB mediated gene expression and CREB gene expression. Results from examples above indicate that a parallel situation may exist in diabetes where the vessel wall is chronically exposed to an environment of hyperglycemia and hyperinsulinemia. In Example 5, a decrease was observed in total CREB protein in vessel walls from insulin resistant and diabetic animals. Example 7 also demonstrated a decrease in CREB protein content and an increase in ICER expression in SMC exposed to HG and HI in culture. Therefore, it is important to fully characterize these changes in SMC in culture and in animal models of diabetes.

In the following experiments, SMC in culture will be exposed to HG and HI and assessed for CREB content and phosphorylation, and ICER content over time. In addition, aortic SMC from control, insulin deficient and insulin resistant rat models of diabetes (STZ, Zucker and Zucker DM) will be assessed for the above. Finally, histological sections from control, insulin resistant and diabetic rat aortas will be assessed for distribution of CREB, PCREB, ICER, and SMC differentiation markers.

SMCs in culture will be exposed to HG and HI and assessed for CREB content, phosphorylation, and ICER content. BASMC are prepared from aortic tissue as described above. Cells are cultured in Growth Medium, consisting of Minimal Essential Medium (Sigma) containing 1× Non-Essential Amino Acids, 0.4 mM glutamine, and 10% Fetal Bovine Serum (Gemini Bio-Products Inc.). Cells are passaged following trypsinization and plated for experiments at densities indicated below. RASMC are isolated from aortic tissue harvested from eight adult Sprague-Dawley rats. Aortic tissue is minced and cells released by collagenase dissociation (7000 U/ml in MEM Eagle's Medium). Dissociated SMC are plated on 35 mm tissue culture-treated culture dishes. Each preparation yields approximately 20 plates of RASMC. Cells are maintained in growth medium and passaged as described for BASMC above.

For these studies, SMCs will be cultured in media with high concentrations of glucose (5-25 mM) and insulin(3-100 nM). Cells will be exposed to these conditions for 24 hours to 10 days and analyzed using immunohistochemistry and Western analysis for the content and phosphorylation of CREB and for ICER content. It is anticipated that there will be a decreased CREB content, preliminary studies suggest that this will take 2-7 days. The induction of ICER will also be examined. A parallel series of experiments will be conducted with thoracic aortas from control, STZ rats and Zucker rats with and without diabetes to assess the impact of diabetes (insulinopenic and insulin resistant) on vessel wall CREB and ICER content in vivo. For these studies, a time course will be conducted at baseline, 1 wk, 2 wk, 4 wk, 6 wk and 8 wk in control and diabetic animals. Functional changes, specifically increased proliferation and migration, have been noted in cultured SMC from STZ rats at 8 weeks that were not present at 2 weeks. Both insulin deficient and insulin resistant rats will be used to examine hyperglycemia and hyperglycemia plus hyperinsulinemia in vivo. While neither animals is a perfect model for diabetes, these studies should provide important new data on the impact of chronic hyperglycemia and hyperglycemia in an insulin resistant state upon CREB and ICER content.

Based on data presented in Example 7, it is anticipated that hyperglycemia will decrease CREB content and induce ICER. High insulin is likely to increase ICER protein with a less significant impact on CREB content. Enhanced migration and proliferation in SMC from these animals is also expected.

### Example 15

The following example provides additional evidence that altering CREB content or function changes SMC phenotype.

CREB is important for differentiation in neurons and adipocytes and essential for maintenance of the mature functional phenotype in cardiac myocytes. Example 8 indicates that increasing WT CREB content in SMC in culture decreases proliferation. The following experiments will explore the phenotypic consequences of altering CREB content and phosphorylation and ICER content on SMC phenotype in culture.

SMC in culture will be stably transfected with inducible dominant negative, constitutively active, WT CREB, sense or antisense ICER, or infected with adenoviral vectors of these genes. These cells will be assessed for: a) proliferation, b) migration, c) contraction and cytoskeletal changes as well as a battery of SMC differentiation markers. For these studies, two approaches for introducing CREB and ICER will be employed. SMC in culture are classically known to be somewhat resistant to transfection protocols. Using Lipofectamine Plus, the present inventors have been able to achieve approximately a 5-15% transfection efficiency and 90-100% adenoviral infection (See Example 8). In the initial experiments, SMCs will be infected with adenoviral dominant negative CREB (adKCREB) or VP-16 CREB (adVP16CREB) and WT CREB (adCREB). Positive clones for the VP16CREB have already been identified and are being purified. Infected cells will be assessed for phenotype and contrasted with adenovirus empty vector transfected cells. The assays to determine phenotype will include: 1) Proliferation: Cell proliferation will be assessed after a 48 hour SS by Thymidine incorporation, MTT, and cell count; additionally, flow cytometry for cell count and assessment of cell cycle phase will be performed (See methods in previous examples); 2) Cells will be transferred to Boyden chambers and assessed for unstimulated and mitogen stimulated migration; 3) Cells will be analyzed for cytoskeletal changes including; actin polymerization, FAK, α SM actin, and β NM actin by immunohistochemistry, and Western analysis for total cell actin (αSM vs. βNM). Additional parallel transfection experiments will be done in cells stably transfected with ecdysome inducible WT CREB, K-CREB and VP-16 CREB and analyzed as above.

It is expected that dominant negative CREB and sense ICER will lead to SMC phenotypic modulation. If the dose delivered overwhelms the cells, apoptosis may occur. Assays to confirm or deny the induction of apoptosis are readily available in the lab in conjunction with the neurodegeneration REAP. Overexpression of WT CREB is likely to promote a differentiated phenotype. The present inventors have already provided data above showing arrest of cell cycle and decreased migration with CREB overexpression. The constitutively active CREB could induce ICER and promote dedifferentiation, promote proliferation by driving immediate early genes, or maintain the mature phenotype. Adenoviral overexpression of constitutively active signaling elements can lead to non-physiological protein interactions. Preliminary transient VP16CREB transient transfections in BASMC demonstrated phenotypic changes consistent with increased differentiation. The dominant negative CREB and ICER overexpression experiments will be the most informative for the question of how CREB down-regulation impacts phenotype.

### Example 16

The following example shows the impact of down-regulation of CREB and induction of ICER impact on CREB dependent transcription of relevant target genes.

The present inventors have determined the ability of insulin and IGF-1 to regulate CREB dependent genes essential for differentiation and survival in adipocyte and neuronal cells lines (Klemm et al., 1998, J. Biol. Chem., 273:917-923; Pugazhenthi, et al., J. Biol. Chem.: (in press), 1998; Pugazhenthi, et al., J. Biol. Chem.: (in press), 1998; Reusch et al., 1995, Endocrinology, 136:2464-2469; Reusch et al., 1994, Endocrinology, 135:2418-2422). β adrenergic stimulation serves this function in SMCS. The present inventors have identified a set of candidate genes, which are dysregulated in atherosclerosis, that contain CREB response elements. Two of these potential candidate genes, vascular endothelial growth factor (VEGF) and inducible nitric oxide (iNOS), have been selected because they have been reported in the literature to be regulated by insulin and dysregulated in atherosclerosis in endothelial cells and SMC (Begum et al., 1998, J. Biol. Chem., 273:25164-70; Hermanson et al., 1997, Brain Res. Mol. Brain Res., 51:188-96; Hoeffler et al., 1990, Mol. Endo., 4:920-930; Horvai et al., 1997, Proc. Natl. Acad. Sci. USA, 94:1074-1079; Hoshiya et al., Hypertension 31:665-671, 1998; Igarashi et al., 1999, J. Clin. Invest., 103:185-95; Iyengar, Science 271:461-463, 1996). If these genes are CREB dependent, then the impact of decreased CREB protein content and blunted active signaling via CREB or ICER induction could alter their transcription. It will be determined whether regulation of VEGF or iNOS is CREB dependent and the impact of HG and HI on their regulation.

First, the impact of isoproterenol (ISO), insulin and IGF-1 on CREB transcriptional activity in SMC will be assessed using CREB responsive reporter systems, VEGF and iNOS promoter-reporter constructs. Additionally, mRNA content of V-EGF and iNOS in cultured SMC will be assessed under all conditions. Next, a parallel series of experiments to those above will be undertaken using chronic HG and HI. Next, the impact of chronic exposure to HG and/or HI upon acute CREB transcriptional activation by ISO, insulin and IGF-1 mediated transcription will be assessed.

The impact of ISO, insulin, and IGF-1 on CREB transcriptional activity will be assessed in SMC. The impact of HG and HI and CREB-TA will be assessed using a number of CRE containing luciferase reporter constructs available in the present inventors' laboratory cotransfected with two different dominant negative CREB isoforms (K-CREB and A-CREB, Charles Vinson, NIH). Next two CRE containing candidate genes, VEGF and iNOS, will be examined to determine whether their transactivation by insulin and IGF-1 is CREB dependent using cotransfection experiments with K-CREB, A-CREB, and ICER. These promoter constructs include: Vascular Endothelial Growth Factor luciferase, VEGF-Luc (Genentec), and inducible Nitrous Oxide Synthase (iNOS)(E. Chang, UC14SC). Regulation of each of these genes is altered in atherosclerosis and/or by diabetes. If VEGF and/or iNOS are CREB dependent the impact of acute and chronic HG and IE will be assessed alone or in combination with acute insulin or IGF-1. ISO and dibuterol cyclic. AMP (dbcAMP) will be used as positive controls to assess CREB dependent transcription in all circumstances. The induction of these genes by the above mentioned agents (ISO, insulin, IGF-1, HG, and IE) will be assessed initially for the full-length promoter constructs. The contribution of CREB will be assessed using K-CREB and A-CREB cotransfected with the full-length promoter constructs. If cotransfection with the dominant negative CREB suggests that CREB is regulating any of these genes, two different strategies will be employed to assess the CREB response element. Initially, deletion mutations of each of the promoters will be used to assess where the CREB responsive element is in each of the promoters. If a CREB response element is located in the area important for CREB responsiveness, that CREB response element will be mutated by site directed mutagenesis to assess the role of CREB in the regulation of any and all of these genes. No examination of the importance of CREB for the regulation of VEGF and iNOS has been published; these studies should yield important new information.

It is anticipated that hyperglycemia and hyperinsulinemia increase the transcription of VEGF and decrease iNOS. VEGF has numerous CREB response elements in its promoter regions and it is likely that the depletion of CREB protein or induction of ICER in diabetes will impact their transcription. It is also conceivable that CREB depletion could alter protein-protein interactions in the nucleus.

### Example 17

The following example shows which pathways activated by high glucose and/or high insulin lead to ICER induction and CREB down-regulation.

HG and HI induce changes in signaling pathways known to regulate CREB and have been indicted as contributors to diabetic vascular dysfunctions. Hyperglycemia increases some isoforms of protein kinase C (PKC). Hyperglycemia also activates some p38 MAPK isoforms resulting in CREB phosphorylation. For hyperglycemia it has been demonstrated that treatment with PKC inhibitors and antioxidants can improve diabetic vascular contractile function in vivo. This phenomenon is likely to be, to some extent, CREB dependent. In the following experiments, the signal transduction pathways that contribute to toxin mediated phosphorylation of CREB, down-regulation of CREB content, and induction of ICER will be assessed. It will also be determined whether inhibitors of PKC and/or p38 NIAPK block these effects. High insulin increases growth factor and fatty acid induced signaling through ras and rho and increases basal ERK activity. It may also impact p38 MAPK activity.

Cultured SMC will be exposed to acute and chronic HG and HI. The signaling pathways contributing to alterations in CREB and ICER will be characterized using a combination of pharmacological inhibitors followed by constitutively active and dominant negative regulators of the p38 MAPK, ERK and PKC signaling pathways. It is anticipated that PKC and p38 MAPK will play a role in CREB regulation by glucose. The impact of augmented signaling through the ERK pathway by FE is less clear. It will be important to determine if these are the primary pathways important for CREB and ICER regulation or if others exist. The results of these signaling studies will serve as the basis for use of specific inhibitors of signal transduction pathways as tools to block phenotypic modulation by diabetes related stimuli.

HG and HI increase CREB phosphorylation in SMC. Initial experiments under this aim will screen for important signaling pathways for this phenomenon using pharmacological inhibitors of P13 kinase, wortmannin, and LY294002; p70 S6 Kinase, rapamycin; MEKI, PD98059; p38 MAPK, SB203580 (Klemm et al., 1998, J. Biol. Chem., 273:917-923; Pugazhenthi et al., 1999, J. Biol. Chem., 274:2829-2837). PKC, GF109203X, LY3791961 (Impey et al., 1998, Neuron, 21:869-883; Salminen et al., 1998, Brain Res. Mol. Brain Res., 61:203-206; Walter et al., 1998, Mol. Cell. Endocrinol., 143; 167-178; and PKA, H89. Read out for these initial experiments will be CREB phosphorylation and CREB transcriptional activity. It is expected that hyperglycemia mediated CREB phosphorylation will be blocked by the PKC P inhibitor probably p38 MAPK inhibitor. Hyperinsulinemia will probably work via ERK½MAPK, but may use additional pathways as is the case for insulin in PC 12 cells. If the data emerge as expected, experiments will be performed to assess whether pharmacological inhibitors, such as the PKC inhibitor LYLY3791961 (Kurt Ways, Lilly) for hyperglycemia can block induction of ICER and down-regulation of CREB content. These experiments should yield important new data as to whether CREB activation is important for its down-regulation.

If these experiments implicate PKC and p38 MAPK, as is expected, the next series of experiments will characterize the PKC and p38 isoforms responsible for CREB down-regulation. In the case of PKC, the literature indicates that PKC β and δ are upregulated in the diabetic vasculature in response to hyperglycemia. PKC δ can drive CREB dependent transcription in PC-12 cells. To examine the importance of PKC β for CREB downregulation, CREB content and ICER in cell lines stably overexpressing PKC β will be assessed. These experiments should define a specific role, if any, for PKC β activation in the down-regulation of CREB. To assess the impact of other PKC isoforms and p38 MAP isoforms, cells will be transiently transfected with active forms (for PKC) or specific isoforms of p38 MAPK with activated upstream kinases (MKK 3 or 6) to assess active impact on CREB dependent transcription and regulation by insulin, IGF-1, HG, and HI. In the proposed model, only those isoforms capable of inducing CREB phosphorylation and transcription which are augmented by HG and/or HI will be further characterized. It is expected that PKC δ and p38 α MAPK to meet these criteria, though others may also. To determine the impact of signaling pathways identified above on CREB and ICER function, the following series of experiments will be undertaken. Cells will be co-transfected with VEGF or iNOS promoter-reporter constructs and PKC or p38 MAPK a. Promoter activity will be assessed under basal conditions and in cells treated with HG or HI for 24, 48, 72 and 96 hours. Acute transcriptional response to insulin, IGF-1 and ISO will also be studied in these co-transfection experiments. If either of these signaling elements down-regulates CREB dependent transcription that element will be further examined by adenoviral infection. For PKC no adenoviral constructs are currently available and would need to be constructed. Cells infected with PKC or p38MAPK (plus MKK3) would be assessed for CREB and ICER protein and mRNA content. If any element leads to CREB down-regulation or induction of ICER, cells would be treated with pharmacological inhibitors prior to infection to see if this could inhibit CREB down-regulation or the induction of ICER.

It is expected that the protein kinase C and p38 inhibitors will block diminution in CREB and the induction of ICER in response to HG. It is also expected that hyperglycemia will increase PKC βII and δ and also p38 MAPK activity (isoform unknown). Chronic elevation of p38 MAPK and PKC would be anticipated to increase CREB phosphorylation which we consider to be the stimulus for induction of ICER and down-regulation of CREB. The mode whereby chronic hyperinsulinemia induces ICER is less clear.

It has been proposed that cAMP acts as a gate for mitogenic signaling by inhibiting ERK MAPK activation and inducing cell cycle arrest. Protracted agonist stimulation, which chronically increases cAMP content, blunts further signaling through CREB by decreasing CREB content and attenuating transcriptional responses to acute stimuli. This has been demonstrated in the setting of β adrenergic stimulation induced cardiomyopathy. One consequence of sustained or high magnitude CREB phosphorylation is the induction of ICER, Inducible cAMP Early Repressor, a CREB-related CREM family transcription factor capable of inhibiting CREB mediated gene expression as well as CREB gene expressions. The present inventors' data indicate that a parallel situation may exist in diabetes where the vessel wall is chronically exposed to an environment of hyperglycemia and hyperinsulinemia.

### Example 18

The following example demonstrates that expression of a dominant negative CREB protein in visceral fat tissue cells leads to the loss of triacylglycerol stores, and a decrease in adipocyte number.

Excessive body weight gain, or obesity is the result of numerous, interacting behavioral, physiological and biochemical factors. One increasingly important factor is the generation of additional fat cells, or adipocytes in response to excess feeding and/or large increases in body fat composition. The generation of new adipocytes is controlled by several “adipocyte-specific” transcription factors that regulate preadipocyle proliferation and adipogenesis. In recent studies, the present inventors have shown that the constitutively expressed transcription factor, CREB, is necessary and sufficient to induce adipogenesis in 3T3-L1 cells in culture. Interestingly, the present inventors have also found that the introduction of a dominant negative CREB protein (KCREB) into mature 3T3-L1 adipocytes results in the complete loss of triacylglycerol stores within three to six days. These data demonstrate a central role for CREB in initiating adipogenesis, and in the maintenance of the mature adipocyte phenotype.

The data in Examples 2-4 demonstrated that two KCREB expressing adipocyte cell lines failed to exhibit signs of differentiation when treated with muristerone prior to and during their exposure to the conventional differentiation-inducing mixture. The ability to block adipogenesis by inhibiting endogenous CREB activity indicated that CREB is required to induce normal adipose differentiation. In addition, Examples 2-4 showed that expression of KCREB before and during the application of the conventional differentiation-inducing mixture completely blocked the appearance of both PPARγ2 and FABP in the day 10 samples. In addition, when the two cell lines, stably transfected with the inducible KCREB expression system, were differentiated to mature, lipid-containing adipocytes by treatment with insulin, Bt2cAMP, and dexamethasone, and then treated with muristerone in media containing insulin, the treated cells completely lost their triacylglycerol vesicles within three to six days, even in the continued presence of insulin and high glucose levels. Thus, CREB not only initiates adipogenesis, but appears to play a key role in maintaining the mature adipocyte phenotype.

In the following experiments, KCREB is delivered to visceral adipose tissue cells in normal and obese mice via an adenovirus vector. It is believed that KCREB expression in these cells will decrease triacylglycerol stores in obese mice, and prevent fat storage in normal animals.

The ability of KCREB to decrease triacylglycerol storage in visceral adipose tissue cells in obese mice will be evaluated via intraperitoneal injection of an adenovirus-KCREB vector. For these experiments, two models of obesity have been selected: (1) a diet-induced model using AKR/J mice; and (2) a genetic model using obese, leptin-deficient C57/B16J ob-/ob- mice. A number of species of mice exhibit diet-induced obesity, but AKR/J mice were selected because they show substantial fat deposition in the visceral adipose tissue depot which is adjacent to our delivery site and easy to evaluate (West et al., 1992, Am. J. Physiol., 262(6 Pt 2):R1025-1032). Ob-/ob- mice were selected as the genetic model, as it is well characterized and widely employed as a conventional genetic model of obesity. Two models will be used to account for any model specific factors, and provide complimentary data from two systems.

The KCREB is delivered to the abdominal adipose depots using an adenovirus vector injected into the peritoneal cavity adjacent to this tissue. In future studies, the role of CREB in adipose tissue function will be assessed through a variety of methods including transgenic mice. However, the proposed technology using adenovirus vectors makes use of reagents, namely adenovirus-KCREB and adenovirus-LacZ (control) which have already been generated and characterized in cell culture. They are therefore the most inexpensive and rapid means with which to perform these experiments. Moreover, several groups (Flint, 1998, Biochem. Biophys. Res. Comm., 252:263-268; Flint et al., 1986, Int'l J. Obesity, 10:69-77; Panton et al., 1990, Am. J. Physiol., 258:E985-E989; Wright et al., 1995, Obesity Res., 3:265-272) have demonstrated that reagents like antibodies can be delivered to the abdominal fat depot via intraperitoneal injection producing decreases in abdominal fat storage and tissue cellularity. Likewise, recent experiments have shown efficient delivery of proteins like β-galactosidase to adipocytes in animals using adenoviral vectors (Levine et al., 1998, J. Nutr. Sci. Vitaminol., 44:569-572). Thus, these studies make sue of available reagents, and are supported by experiments from several other labs indicating the ability to deliver material to adipose tissue.

Following injection of the adenovirus vectors, various biochemical, morphological, and behavior parameters are measured to evaluate the effectors of KCREB. The animals are followed for 14 days following injection of the viral vectors, which is believed to be sufficient time in which to observe an effect of KCREB expression. At the end of the 14 day treatment period, animals are sacrificed and individual fat depots removed by dissection and weighed. Although changes in visceral fat tissue are the focus of these studies, the other fat depots are evaluated since changes in one fat depot may produce changes in other sites. For example, treatment of mice with leptin typically decreases the visceral adipose pool, but increase subcutaneous fat stores. A small portion of visceral adipose tissue is digested with collagenase, stained with oil red, and visualized by light microscopy to determine adipocyte size. Whole body lipid content is measured by digestion of the animals in ethanol-KOH. Blood glucose, insulin, free fatty acids, triglycerides, and leptin levels will be determined. Mice are weighed daily during the 14 day treatment period as an indirect indicator of body composition changes. Chow weight is measured daily as an indicator of feeding behavior, and to determine caloric intake.

Several controls are employed in these experiments. First, lean control animals are included to determine whether KCREB influences weight gain or loss, or other parameters in lean animals. For the diet-induced model, animals fed synthetic, high carbohydrate chow serves as the lean controls. For the genetic model, C57/B16J ob-/ob mice are used which do not become obese on a normal diet. To control for the effects of manipulation, stress and discomfort, some animals are injected with phosphate buffered saline (PBS, the injection vehicle) alone. Another control consists of animals injected with adenovirus-LacZ, from which β-galactosidase is expressed. This protein should have no effect on any parameter, and this vector allows control for non-specific virus effects like immune responses or the action of viral proteins. To ensure that sufficient virus is introduced to produce response, but prevent any confounding effects, multiple doses of virus are tested. Based on research reports in the scientific literature, virus doses of 0.5, 1.5 and 4.0×10⁶ plaque forming units per animal will be used.

It is believed that introduction of KCREB will decrease adipose triacylglycerol stores in obese mice. The stringent controls and number of parameters measured allow the drawing of meaningful conclusions from these experiments.

Detailed Materials and Methods for This Experiment:

Two models of obesity, a monogenetic model and a diet-induced model are employed in these studies. The monogenetic model will consist of mice of the genotype C57/B16J ob-/ob-, with C57/B16J ob-/ob mice serving as the control. For the diet-induced model, AKR/J mice fed a synthetic high fat/calorie chow (Research Diets, Inc. D12344) are employed. AKR/J mice fed synthetic, high carbohydrate chow (D11724), ad libitum, will serve s the controls for the diet-induced obesity experiments. AKR/J mice were selected because they exhibit increases adiposity in all body fat depots (West et al., 1992, Am. J. Physiol., 262(6 Pt 2):R1025-1032).

Male ob-/ob- and ob-/ob mice are purchased from Jackson Laboratories. Six mice are used for each treatment (see below) which is the minimum number conventionally employed to generate statistically relevant data. All mice are housed in individual cages. Untreated mice are fed synthetic, high carbohydrate chow (D1724) for 30 days to allow the mice to acclimatize to their surroundings and gain weight. After the 30 day acclimatization period, mice are treated as described below, and subjected to a series of tests described in following sections. Any animals exhibiting lethargy, rapid/difficulty breathing, ruffled fur or ulcerative dermatitis, diarrhea/constipation, or bleeding are immediately euthanized via metafane inhalation to unconsciousness followed by decapitation with a small animal guillotine. Weight loss is evaluated, but not used to determine health status of the animals, as this is one of the experimental parameters to be tested.

Male AKR/J mice are purchased from Jackson Laboratories, and housed as described above for the ob-/ob- ob/ob mice. Control animals are fed synthetic, high carbohydrate chow, and “experimental” mice fed Diet 12344 (high fat/calorie diet from Research Diets, Inc.) ad libitum to induce obesity throughout the acclimatization and study period.

Treatments:

Mice are randomly separated into groups of six animals each. Each group is subjected to intraperitoneal injection of up to 1 ml of PBS alone or PBS containing various adenovirus vectors via a 27 gauge, half-inch, short bevel needle attached to a 1 cc tuberculin syringe. Control animal will receive PBS alone, or PBS containing 0.5×10⁶, 1.5×10⁶, or 4×10⁶ plaque forming units (pfu) of Ad-LacZ, an adenovirus vector from which β-galactosidase protein is expressed. This protein should not have any effects on the animals, and has been used as a control protein in numerous other studies. “Experimental” animals are injected with PBS alone or PBS containing 0.5×10⁶, 1.5×10⁶, or 4×10⁶ pfu of Ad-KCREB, an adenovirus vector from which the dominant negative CREB protein, KCREB, is expressed. The treatment regimens are:

| | |
|---|---|
| AKR/J - Normal Chow (Control) | |
| PBS (mock or control) | |
| 0.5 × 10⁶ Ad-LacZ | 0.5 × 10⁶ Ad-KCREB |
| 1.5 × 10⁶ Ad-LacZ | 1.5 × 10⁶ Ad-KCREB |
| 4.0 × 10⁶ Ad-LacZ | 4.0 × 10⁶ Ad-KCREB |
| AKR/J - Diet 12344 | |
| PBS (mock or control) | |
| 0.5 × 10⁶ Ad-LacZ | 0.5 × 10⁶ Ad-KCREB |
| 1.5 × 10⁶ Ad-LacZ | 1.5 × 10⁶ Ad-KCREB |
| 4.0 × 10⁶ Ad-LacZ | 4.0 × 10⁶ Ad-KCREB |

- OR -

| | |
|---|---|
| Ob−/ob Control | |
| PBS (mock or control) | |
| 0.5 × 10⁶ Ad-LacZ | 0.5 × 10⁶ Ad-KCREB |
| 1.5 × 10⁶ Ad-LacZ | 1.5 × 10⁶ Ad-KCREB |
| 4.0 × 10⁶ Ad-LacZ | 4.0 × 10⁶ Ad-KCREB |
| Ob−/Ob− | |
| PBS (mock or control) | |
| 0.5 × 10⁶ Ad-LacZ | 0.5 × 10⁶ Ad-KCREB |
| 1.5 × 10⁶ Ad-LacZ | 1.5 × 10⁶ Ad-KCREB |
| 4.0 × 10⁶ Ad-LacZ | 4.0 × 10⁶ Ad-KCREB |

Experimental Parameters:

The following parameters are measured to evaluate the effect of KCREB expression on murine obesity.

Chow Weight:

During the 14 day experimental period, chow weight is measured daily to follow feeding patterns and caloric intake.

Body Weight:

Each mouse is weighed daily, to follow the effects of KCREB expression on total body weight.

Blood Glucose, Insulin, Free Fatty Acids, Triglycerides, and Leptin Levels:

Following an overnight fast, animals are anesthetized with avertin on day 14. One ml of blood is obtained from the inferior vena cava, and the mouse sacrificed by cervical dislocation. Blood glucose and insulin levels are measured as markers of insulin action. Blood free fatty acids, triglycerides, and leptin levels will also be evaluated. All tests are performed by the Metabolic Core Laboratory at the Center for Human Nutrition.

KCREB Distribution and Expression Levels:

As an initial assessment of KCREB expression and tissue distribution, samples of is brain, heart, lung, kidney, liver, spleen, skeletal muscle, and abdominal and gonadal fat tissue is recovered from animals following euthanasia. Samples are homogenized, and proteins separated on SDS-PA gels, and subjected to Western blot analysis using antibodies to FLAG epitope (KCREB is FLAG-tagged). Similarly, tissues from animals treated with AD-LacZ is assayed for β-galactosidase levels.

Adipose Tissue Cellularity and Cell Morphology:

Small portions of mesenteric fat tissue is recovered from euthanized animals, digested with collagenase, and stained with oil red O. Stained cells are evaluated for adipocyte cell number, cell size, and triacylglycerol content.

Adipose Tissue Weight:

Differences in adipose tissue weight between experimental and control animals are defined by removal of the gonadal, retroperitoneal, inguinal, mesenteric, and intrascapular fat depots from sacrificed mice by dissection. Individual fat depots will then be weighed.

Body Fat Composition:

Sacrificed animals will be “digested,” and fats saponified in ethanol/KOH at 60° C. for 3 days. An aliquot of this material is clarified by the addition of MgCl₂, a brief period of refrigeration, and centrifugation at 10,000×g for 10 min. A portion of the supernatant is then diluted with water and subjected to enzymatic triglyceride analysis. Triglyceride concentration will then be used to calculate percent body fat for each animal.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

What is claimed is:

1. A method to modulate the phenotype of a target cell population in a patient, comprising administering to a patient a composition comprising a recombinant nucleic acid molecule comprising a nucleic acid sequence encoding a cyclic-AMP responsive element binding (CREB) protein having CREB biological activity operatively linked to a transcription control sequence;
wherein said CREB protein is expressed by said recombinant nucleic acid molecule in target cells in said patient, said target cells being selected from the group consisting of:
a. cells deficient in endogenous CREB expression;
b. cells deficient in endogenous CREB biological activity; and,
c. cells having normal endogenous CREB expression and biological activity which are predisposed to become deficient in endogenous CREB expression or biological activity;
wherein said expression of said CREB protein in said target cells is sufficient to modulate the phenotype of said cells.

2. The method of claim 1, wherein said CREB protein having CREB biological activity is selected from the group consisting of:
a. a CREB protein having wild-type CREB biological activity; and,
b. a CREB protein having constitutively active CREB biological activity.

3. The method of claim 1, wherein said CREB protein having CREB biological activity is selected from the group consisting of wild-type CREB protein, ATF-1, VP16-CREB, and CREB DIEDML.

4. The method of claim 1, wherein said CREB protein having CREB biological activity is encoded by a nucleic acid sequence that hybridizes under stringent hybridization condition to a nucleic acid sequence selected from the group consisting of SEQ ID NO:1, SEQ ID NO:3, SEQ ID NO:5 and SEQ ID NO:20.

5. The method of claim 1, wherein said CREB protein having CREB biological activity comprises an amino acid sequence selected from the group consisting of SEQ ID NO:2, SEQ ID NO:4, SEQ ID NO:6, SEQ ID NO:21, an amino acid sequence comprising a biologically active fragment of SEQ ID NO:2, an amino acid sequence comprising a biologically active fragment of SEQ ID NO:4, an amino acid sequence comprising a biologically active fragment of SEQ ID NO:6, and an amino acid sequence comprising a biologically active fragment of SEQ ID NO:21.

6. The method of claim 1, wherein said nucleic acid sequence is selected from the group consisting of SEQ ID NO:1, SEQ ID NO:3, SEQ ID NO:5, SEQ ID NO:20, a fragment of SEQ ID NO:1 encoding a biologically active CREB protein, a fragment of SEQ ID NO:3 encoding a biologically active CREB protein, a fragment of SEQ ID NO:5 encoding a biologically active CREB protein, and a fragment of SEQ ID NO:20 encoding a biologically active CREB protein.

7. The method of claim 1, wherein said recombinant nucleic acid molecule comprises a viral vector.

8. The method of claim 7, wherein said viral vector is from a virus selected from the group consisting of alphaviruses, poxviruses, adenoviruses, herpesviruses, lentiviruses, adeno-associated viruses and retroviruses.

9. The method of claim 7, wherein said viral vector is from an adenovirus.

10. The method of claim 1, wherein said composition further comprises a liposome delivery vehicle that delivers said recombinant nucleic acid sequence into said cell.

11. The method of claim 10, wherein said liposome delivery vehicle comprises lipids selected from the group consisting of small unilamellar vesicle lipids, multilamellar vesicle lipids and extruded lipids.

12. The method of claim 10, wherein said liposome delivery vehicle comprises cationic liposomes.

13. The method of claim 10, wherein said liposome delivery vehicle comprises lipids selected from the group consisting of DOTMA, DOTAP, DOTIM, DDAB and cholesterol.

14. The method of claim 10, wherein said liposome delivery vehicle comprises a targeting agent that specifically binds to a molecule on the surface of said target cells.

15. The method of claim 14, wherein said targeting agent is selected from the group consisting of an antibody, a soluble receptor, and a ligand.

16. The method of claim 1, wherein said recombinant nucleic acid molecule is administered to said patient as naked DNA.

17. The method of claim 1, wherein said composition is administered by a route of administration selected from the group consisting of ex vivo delivery, intravenous administration, intraperitoneal administration, intramuscular administration, intracoronary administration, intraarterial administration, subcutaneous administration, transdermal delivery, intratracheal administration, subcutaneous administration, intraarticular administration, intraventricular administration, inhalation, intracerebral, nasal, oral, pulmonary administration, impregnation of a catheter, and direct injection into a tissue.

18. The method of claim 1, wherein said CREB protein is expressed in said cell at a level of at least about 10,000 molecules of CREB protein per cell.

19. The method of claim 1, wherein said CREB protein is expressed in said cell at a level of at least about 25,000 molecules of CREB protein per cell.

20. The method of claim 1, wherein said CREB protein is expressed in said cell at a level of at least about 50,000 molecules of CREB protein per cell.

21. The method of claim 1, wherein said transcription control sequence comprises a target cell-specific promoter.

22. The method of claim 21, wherein said promoter is inducible.

23. The method of claim 1, wherein said patient has or is at risk of developing diabetes.

24. The method of claim 23, wherein said target cells are selected from the group consisting of adipocytes, vascular smooth muscle cells, cardiomyocytes, hepatocytes, skeletal muscle cells, beta cells, pituitary cells, synovial lining cells, ovarian cells, testicular cells, fibroblasts, endothelial cells, and neural cells.

25. The method of claim 23, wherein said target cells have decreased CREB expression prior to said step of administering.

26. The method of claim 23, wherein said target cells are adipocytes, and wherein said expression of said CREB protein results in a change in expression of a protein in said adipocytes selected from the group consisting of an increase phosphoenolpyruvate carboxykinase (PEPCK), an increase Glut4, an increase in PPAR γ, an increase in fatty acid synthetase (FAS), an increase in fatty acid binding protein (FABP), an increase in C/EBP α, an increase in C/EBP β, an increase in LPL and a decrease in PREF-1.

27. The method of claim 26, wherein expression of said CREB protein in said adipocytes produces a result selected from the group consisting of decreased insulin resistance, normalized glucose control, and normalized lipid handling.

28. The method of claim 26, wherein said step of administering is by a route selected from the group consisting of intravenous administration, intraarterial administration, intraperitoneal administration and fat deposit injection.

29. The method of claim 26, wherein said transcription control sequence comprises a promoter selected from the group consisting of aP2 promoter, lipoprotein L (LPL) promoter and leptin promoter.

30. The method of claim 23, wherein said target cells are vascular smooth muscle cells, and wherein said expression of said CREB protein modulates phenotypic characteristics in said vascular smooth muscle cells selected from the group consisting of decreased proliferation of said cells, decreased migration of said cells, inhibition of cell cycle entry by said cells, increased contractility, decreased synthetic function and decreased cytokine expression.

31. The method of claim 30, wherein said expression of said CREB protein results in a change in expression of a protein in said vascular smooth muscle cell selected from the group consisting of a decrease in vascular endothelial growth factor (VEGF), an increase in endothelial nitric oxide synthase (eNOS), an increase in tissue-plasminogen activator (tPA), a decrease in plasminogen activator inhibitor-1 (PAI-1), a decrease in heparin binding-endothelial growth factor (HB-EGF), and a decrease in inducible nitric oxide synthase (iNOS).

32. The method of claim 30, wherein said expression of said CREB protein in said vascular smooth muscle cells produces a result in said patient selected from the group consisting of decreased susceptibility to post-angioplasty restenosis, reduced vessel occlusion, reduced atherosclerosis plaque formation, and decreased potential for pulmonary hypertension.

33. The method of claim 30, wherein said transcription control sequence comprises a promoter selected from the group consisting of α smooth muscle actin promoter and α smooth muscle myosin promoter.

34. The method of claim 30, wherein said step of administering is by a route selected from the group consisting of intracoronary administration, intravenous administration, impregnation of angioplasty catheter, intraarterial administration, and pulmonary administration.

35. The method of claim 30, wherein said step of administering is performed concurrent with or following angioplasty.

36. The method of claim 1, wherein said patient has or is at risk of developing a condition selected from the group consisting of atherosclerosis, angina, acute myocardial infarction, stroke, pulmonary hypertension, amputation from peripheral vascular disease, and post-angioplasty restenosis.

37. The method of claim 36, wherein said target cells are vascular smooth muscle cells, and wherein said expression of said CREB protein modulates phenotypic characteristics in said vascular smooth muscle cells selected from the group consisting of decreased proliferation of said cells, decreased migration of said cells, inhibition of cell cycle entry by said cells, increased contractility, decreased synthetic function and decreased cytokine expression.

38. The method of claim 36, wherein said expression of said CREB protein results in a change in expression of a protein in said vascular smooth muscle cell selected from the group consisting of a decrease in n vascular endothelial growth factor (VEGF), an increase in endothelial nitric oxide synthase (eNOS), an increase in tissue-plasminogen activator (tPA), a decrease in plasminogen activator inhibitor-1 (PAI-1), a decrease in heparin binding-endothelial growth factor (HB-EGF), and a decrease in inducible nitric oxide synthase (iNOS).

39. The method of claim 36, wherein said expression of said CREB protein in said vascular smooth muscle cells produces a result in said patient selected from the group consisting of decreased susceptibility to post-angioplasty restenosis, reduced vessel occlusion, reduced atherosclerosis plaque formation, and decreased potential for pulmonary hypertension.

40. The method of claim 36, wherein said transcription control sequence comprises a promoter selected from the group consisting of α smooth muscle actin promoter and α smooth muscle myosin promoter.

41. The method of claim 36, wherein said step of administering is by a route selected from the group consisting of intracoronary administration, intravenous administration, impregnation of angioplasty catheter, intraarterial administration, and pulmonary administration.

42. The method of claim 1, wherein said patient has or is at risk of developing heart failure.

43. The method of claim 42, wherein said target cells are cardiomyocytes, and wherein expression of said CREB protein in said cardiomyocytes modulates phenotypic characteristics in said cardiomyocytes selected from the group consisting of expression of α-myosin heavy chain (α-MHC), spontaneous contraction, myocyte size, vacuolation and fibrosis.

44. The method of claim 42, wherein said expression of said CREB protein in said cardiomyocytes results in decreased characteristics associated with dilated cardiomyopathy in said patient.

45. The method of claim 42, wherein said transcription control sequence comprises a promoter selected from the group consisting of α-myosin heavy chain promoter, cardiac myosin light chain-2 promoter, β-myosin heavy chain promoter, cardiac troponin I promoter and cardiac troponin T promoter.

46. The method of claim 42, wherein said step of administering is by a route selected from the group consisting of intracoronary administration, intraventricular injection, intraarterial administration and intravenous administration.

47. The method of claim 1, wherein said patient has or is at risk of developing pulmonary hypertension.

48. The method of claim 47, wherein said target cells are vascular smooth muscle cells.

49. The method of claim 1, wherein said patient has or is at risk of developing osteoarthritis.

50. The method of claim 49, wherein said target cells are synovial lining cells.

51. The method of claim 50, wherein said step of administering is by a route selected from the group consisting of injection into a joint where osteoarthritis is or may occur, intravenous administration, intraarticular administration, and intraarterial administration.

52. The method of claim 1, wherein said target cells are neural cells.

53. The method of claim 52, wherein said transcription control sequence comprises a promoter selected from the group consisting of chromogranin A promoter, chromogranin B promoter Thy-1 promoter, and vgf promoter.

54. The method of claim 52, wherein said transcription control sequence comprises neuron restrictive enhancer elements.

55. The method of claim 52, wherein said patient has a spinal cord transsection.

56. The method of claim 52, wherein said patient has or is at risk for developing acute neuronal ischemia.

57. The method of claim 52, wherein said patient has or is at risk of developing Alzheimer's disease.

58. The method of claim 57, wherein said target cells are hippocampal neurons.

59. The method of claim 52, wherein said patient has or is at risk of developing Parkinson's disease.

60. The method of claim 59, wherein said neural cells are dopaminergic neural transplant cells, and wherein said step of administering comprises ex vivo delivery of said composition to said dopaminergic neural transplant cells, followed by transplantation of said dopaminergic neural transplant cells into said patient.

61. The method of claim 52, wherein said patient has or is at risk of developing depression.

62. The method of claim 61, wherein said target cells are cells of the cortex and basal ganglia.

63. The method of claim 1, wherein said composition further comprises a pharmaceutically acceptable excipient.

64. A method for restoring the ability of a cell to differentiate, comprising transfecting said cell deficient in CREB expression or CREB biological activity with a recombinant nucleic acid molecule comprising a nucleic acid sequence encoding a cyclic-AMP responsive element binding (CREB) protein having CREB biological activity such that said CREB protein encoded by said recombinant nucleic acid molecule is expressed in said cell, wherein prior to said step of transfecting, said cell is not fully differentiated.

65. A method to treat diabetes in a patient, comprising administering to said patient a composition comprising a recombinant nucleic acid molecule comprising a nucleic acid sequence encoding a cyclic-AMP responsive element binding (CREB) protein having CREB biological activity operatively linked to a transcription control sequence;
wherein said CREB protein is expressed by said recombinant nucleic acid molecule in target cells in said patient, said target cells being selected from the group consisting of adipocytes and vascular smooth muscle cells;
wherein said expression of said CREB protein in said target cells is sufficient to modulate the phenotype of said cells.

66. The method of claim 65, wherein expression of said CREB protein in said target cells produces a result in said patient selected from the group consisting of increased glucose control, decreased insulin resistance, reduced post-angioplasty restenosis, reduced atherosclerosis, reduced total body adiposity, normalization of lipid handling and normalization of hepatic glucose and protein handling.

67. A method to inhibit tumor neovascularization in a patient, comprising administering to said patient a composition comprising a recombinant nucleic acid molecule comprising a nucleic acid sequence encoding a cyclic-AMP responsive element binding (CREB) protein having dominant negative CREB biological activity operatively linked to a transcription control sequence;
wherein said CREB protein is expressed by said recombinant nucleic acid molecule in fibroblasts and endothelial cells in or near a tumor in said patient; and,
wherein the expression of said CREB protein in said fibroblasts and endothelial cells is sufficient to modulate the phenotype of said fibroblasts and endothelial cells, resulting in inhibition of tumor neovascularization in said patient.

68. The method of claim 67, wherein said CREB protein having dominant negative CREB biological activity is selected from the group consisting of KCREB, A-CREB, CREB M1, and a CREB DNA-binding region.

69. The method of claim 67, wherein said CREB protein having dominant negative biological activity comprises an amino acid sequence selected from the group consisting of SEQ ID NO:6 and an amino acid sequence comprising a biologically active fragment of SEQ ID NO:6.

70. The method of claim 67, wherein said nucleic acid sequence is selected from the group consisting of SEQ ID NO:5 and a fragment of SEQ ID NO:5 encoding a CREB protein having dominant negative CREB biological activity.

71. A method to decrease total body adiposity, comprising administering to said patient a composition comprising a recombinant nucleic acid molecule comprising a nucleic acid sequence encoding a cyclic-AMP responsive element binding (CREB) protein having dominant negative CREB biological activity operatively linked to a transcription control sequence;
wherein said CREB protein is expressed by said recombinant nucleic acid molecule in adipocytes of said patient; and,
wherein the expression of said CREB protein in said adipocytes is sufficient to inhibit differentiation of said adipocytes, resulting in a decrease in total body adiposity in said patient.
